(19) United States
(12) Patent Application Publication
(10) Pub. No.: US 2009/0192190 A1
(43) Pub. Date: Jul. 30, 2009

(54) Benzoic Acid Derivatives that are Modulators or Agonists of GlyR

(76) Inventors: Helena Gyback, Sodertalje (SE); Markus Haeberlein, Sodertalje (SE); Catrin Jonasson, Sodertalje (SE); Jacob Kihlstrom, Sodertalje (SE); Hakan Molin, Sodertalje (SE); Berit Molin, Sodertalje (SE); Niklas Plobeck, Sodertalje (SE); Andreas Hettman, Sodertalje (SE); Jenny Viklund, Sodertalje (SE); Ulrika Yngve, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 11/912,552

(22) PCT Filed: May 8, 2006
(86) PCT No.: PCT/SE06/00547

§ 371 Date: Jun. 9, 2008

(30) Foreign Application Priority Data
May 9, 2005 (SE) 0501058-2

(51) Int. Cl. A61K 31/47; C07C 65/01; A61K 31/192; A61K 31/44; C07D 213/70; C07D 215/14; A61P 25/00

(52) U.S. Cl. 514/311; 562/474; 514/568; 514/345; 546/301; 546/174

(57) ABSTRACT

Compounds of formula I,

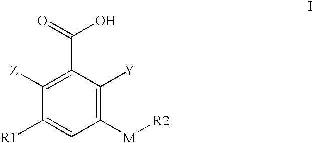

wherein R1, Z, Y, M and R2 are as defined in the specification, pharmaceutically acceptable salts thereof, pharmaceutical composition containing the same, methods of using the same for therapeutic purposes and methods of making the same.

### FIELD OF THE INVENTION

The present invention relates to new compounds of formula I, as a free acid or a pharmaceutically acceptable salt, solvate or solvate of salt thereof. The present invention also relates to use of such compounds in therapy, and also pharmaceutical formulations containing such compounds. The present invention further relates to a process for the preparation of compounds of formula I.

### BACKGROUND OF THE INVENTION

The inhibitory glycine receptors (GlyRs) are ion channels belonging to the cys-loop ligandgated ion channel family. They are pentameric structures composed of two types of membrane spanning subunits (α and β) forming a pore that is permeable to anions. The subunits have four transmembrane domains and a large extracellular N-terminus. Four distinct α subunits (α1 (Pfeiffer, F, H Betz. Brain Research 226, 273-9. 1981); (Pfeiffer et al Journal of Biological Chemistry 257, 9389-93. 1982), α2 (Becker et al EMBO Journal 7, 3717-26. 1988); (Akagi, H, K Hirai, F Hishinuma. FEBS Letters. 281, 160-6. 1991; Kuhse, J, V Schmieden, H Betz, 1990a, Neuron, v. 5, p. 867-73), α3 (Kuhse, J, V Schmieden, H Betz, 1990b, J Biol Chem, v. 265, p. 22317-20) α4 (Harvey, et al, European Journal of Neuroscience 12, 994-1001. 2000)) and one β3 subunit (Pfeiffer and Betz, 1981), (Pfeiffer et al., 1982) have been identified. All subunits except α4 do appear to exist in humans. The predominant receptor isoform consists of α1- and β-subunits with a possible stoichiometry 3α2β. In recombinant systems, homo-oligomeric α-subunits (homomeric GlyR α1) function efficiently with functional properties similar to those of native receptors.

GlyRs are located at postsynaptic membranes mainly in the spinal cord and brain stem (Rajendra, S, J W Lynch, P R Schofield. Pharmacology & Therapeutics 73, 121-46. 1997); (Laube, B, G Maksay, R Schemm, H Betz. Trends in Pharmacological Sciences 23, 519-527. 2002). Glycinergic neurons in the dorsal horn receive a major input from myelinated low-threshold mechanoreceptive primary (Aβ) afferents. Binding of an agonist induces rapid opening of the channel and allowing an influx of Cl⁻ into the cytoplasm. The following hyperpolarisation of the postsynaptic membrane stabilises the resting potential of the cell and thus inhibits neuronal firing. It has been suggested that loss of this inhibitory modulation, as might occur following peripheral or central nerve injury, could facilitate synaptic connections between Aβ-fibers and pain-signalling pathways, thereby resulting in the miscoding of this input as pain. This has been modelled experimentally in animals by the spinal administration of the specific glycine receptor antagonist strychnine (Sorkin, L S, S Puig. Pain 68, 283-92. 1996); (Sherman, S E, C W Loomis. Pain 56, 17-29. 1994); (Sherman, S E, C W Loomis. Canadian Journal of Physiology & Pharmacology 73, 1698-705. 1995; Sherman, S E, C W Loomis. Pain 66, 321-330. 1996); (Yaksh, T L, 1989, Pain, v. 37, p. 111-23); (Beyer, C, C Banas, P Gomora, B R Komisaruk. Pharmacology, Biochemistry & Behavior 29, 73-8. 1988); (Onaka, M, T Minami, I Nishihara, S Ito. Anesthesiology 84, 1215-22. 1996);

Further, it has been shown that mice deficient in GlyR α3 show a reduction in pain sensitisation induced by spinal PGE2 injection or peripheral inflammation. GlyR α3 deficient mice do also lack PGE2 induced inhibition of glycinergic neurotransmission (Harvey, R J, U B Depner, H Wassle, S Ahmadi, C Heindl, H Reinold, T G Smart, K Harvey, B Schutz, O M Abo-Salem, A Zimmer, P Poisbeau, H Welzl, D P Wolfer, H Betz, H U Zeilhofer, U Muller. Science 304, 884-887. 2004).

Positive modulators or agonists of GlyR could be therapeutically beneficial in all conditions with impaired inhibitory tone, specifically as analgesics in neuropathic or inflammatory pain syndromes, such as painful diabetic neuropathy, post traumatic neuralgia, post herpetic neuralgia, trigeminal neuralgia, arthritis, rheumatoid diseases, fibromyalgia, low back pain with radiculopathy and post-operative pain. Further in pain associated with various conditions including angina, renal or billiary colic, menstruation, migraine and gout, stroke, head trauma, anoxic and ischemic injuries, hypoglycaemia, cardiovascular diseases and cancer. GlyR agonists or positive modulators could also be used as anticonvulsants and muscle-relaxants as well as anti-inflammatory agents.

Glycine receptors are also involved in the acrosome reaction (AR) and activation of GlyRs seems to be essential for the AR to occur. GlyR agonists or positive modulators could therefore be useful as fertility enhancers or as a male contraceptive. Glycine receptors are also expressed in the auditory pathways and in the retina. GlyR positive modulators or agonists could therefore be used in the treatment of auditory neuropathic disorders such as tinnitus and opthalmological disorders such as retinopathies, diabetic retinopathies and glaucoma (Lynch, J W. Physiol. Rev. 84, 1051-1095. 2004).

Glycine receptor subunits have also been identified in the nucleus accumbens and GlyR selective compounds have been suggested to combat psychiatric disorders, in which the mesolimbic dopamine system is implicated, such as alcoholism, drug addiction and psychosis (Molander, A, B Soderpalm. Alcoholism: Clinical and Experimental Research 29, 17-26. 2005).

Prostaglandins and leukotrienes are produced by the activity of three enzymes; cyclooxygenase-1, cyclooxygenase-2 (COX-1 and COX-2) and 5-lipoxygenase (5-LOX), as part of the arachidonic acid (AA) pathway. COX-1 converts AA to e.g. prostaglandins such as PGD2, PGE2, PGF2 and PGI2 (prostacyclin) and thromboxanes such as TXA2. COX-2 converts AA to a narrower range of prostaglandins, specifically PGE2 and PGI2. 5-LOX together with other enzymes converts AA to leukotienes (LTB4, LTC4, LTD4 and LTE4). The products from the AA pathway play a major role in human physiology that includes renal homeostasis, gastroprotection, vascular homeostasis and pathophysiological processes, such as pain and inflammation.

PGE2 and PGI2 have various physiological and pathophysiological effects. For example they have potent effects on vasodilatation and vascular permeability.

Inhibitors of cyclooxygenases have been developed as anti-inflammatory drugs as have inhibitors of 5-lipoxygenase. Dual COX/LOX inhibitors are in the clinic for evaluation of inflammation related diseases, such as rheumatoid arthritis and osteoarthritis as well as pneumological diseases. They could also be used in arthrosclerosis and stroke. Further they could be used as antihypertensive agents (Simmons, D L, Botting Regina M., T Hla. Pharmacol Rev 56, 387-487. 2004), (Bertolini, A, A Ottani, Sandrini M. Current Medicinal Chemistry 9, 1033-1043. 2002).

### SUMMARY OF THE INVENTION

The object of the present invention is thus to provide new positive modulators and/or agonists of GlyR, that are optionally also COX and/or LOX inhibitors.

Accordingly, the present invention provides compounds of formula I, or pharmaceutically acceptable salts thereof

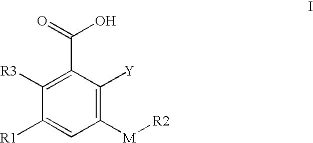

wherein
Y is selected from hydrogen, —OH, halo, —OC₁₋₆alkyl, and —C₁₋₆alkyl, the two latter optionally substituted with halo, —CN, —OH, —CF₃, —NH₂;
R1 is selected from —C₃₋₆cycloalkyl, heterocycloalkyl, aryl, alkylaryl, heteroaryl, and —C₃₋₆-alkyl, optionally substituted with halo, —CN, —OH, —CF₃, —OCF₃, —NH₂, —CONH₂;
M is selected from —C(O)—, —C(H₂)—, —CH(OR^{a})—, —N(OH)—, —N(R^{a})—, —S(O)ᵣ—, heteroaryl and a bond; wherein R^{a} is hydrogen or C₁₋₆alkyl and r is 0, 1 or 2;
R2 is either selected from hydrogen, halo, —CN, or is a group D selected from —C₁₋₆alkyl, C₃₋₆cycloalkyl, heterocycloalkyl, —N(CH₃)₂, aryl, alkylaryl, heteroaryl, and heterocyclic groups;
where D is optionally substituted with one or more substituents G selected from halo, —NO₂, —CN, —OH, —CF₃, —OCF₃, —NH₂, —CONH₂, —COOH, aryl, heteroaryl, heterocyclic groups, —C₁₋₆alkyl, —C₁₋₆alkoxy, heterocycloalkyl, and C₁₋₆alkylcarboxylate;
where D may optionally be connected to G by a linker group L selected from —C(O)—, —S—, and —S(O₂)—;
and G, if substitutable, is optionally further substituted with one or more substituents selected from halo, —NO₂, —CN, —OH, —CH₃, —OCH₃, —CF₃, —OCF₃, —NH₂, —CONH₂, —COOH, and C₁₋₆alkylcarboxylate;
and R3 is selected from —OH and C₁₋₆alkoxy;
provided that when M is a bond and R3 is —OH, then R2 is not —C₁₋₆alkyl,
and that when M is —C(O)— then R2 is not hydrogen or —CH₃,
and with the proviso that the compound is not
2-hydroxy-3-isopropyl-6-methyl-5-[(4-nitrophenyl)sulfinyl]benzoic acid,
2-hydroxy-3-isopropyl-6-methyl-5-[(4-nitrophenyl)sulfonyl]benzoic acid,
2-hydroxy-3-isopropyl-6-methyl-5-[(4-nitrophenyl)thio]benzoic acid,
2-hydroxy-3-methyl-5-[(4-methylphenyl)sulfonyl]benzoic acid,
2-hydroxy-3-methyl-5-[(4-nitrophenyl)sulfinyl]benzoic acid,
2-hydroxy-3-methyl-5-[(4-nitrophenyl)sulfonyl]benzoic acid,
2-hydroxy-3-methyl-5-[(4-nitrophenyl)thio]benzoic acid,
3-[(4-bromo-3-methylphenyl)sulfonyl]-5-tert-butyl-6-hydroxy-2-methylbenzoic acid,
3-[(4-bromo-3-methylphenyl)thio]-5-tert-butyl-6-hydroxy-2-methylbenzoic acid,
3-[(4-bromophenyl)thio]-5-tert-butyl-6-hydroxy-2-methylbenzoic acid,
3-[(4-chlorophenyl)sulfonyl]-6-hydroxy-5-isopropyl-2-methylbenzoic acid,
3-tert-butyl-2-hydroxy-5-[(4-nitrophenyl)sulfonyl]benzoic acid,
3-tert-butyl-2-hydroxy-5-[(4-nitrophenyl)thio]benzoic acid,
3-tert-butyl-2-hydroxy-6-methyl-5-(phenylsulfonyl)benzoic acid,
3-tert-butyl-2-hydroxy-6-methyl-5-(phenylthio)benzoic acid,
3-tert-butyl-2-hydroxy-6-methyl-5-[(2,4,5-trichlorophenyl)sulfinyl]benzoic acid,
3-tert-butyl-2-hydroxy-6-methyl-5-[(2,4,5-trichlorophenyl)thio]benzoic acid,
3-tert-butyl-2-hydroxy-6-methyl-5-[(2-nitrophenyl)sulfonyl]benzoic acid,
3-tert-butyl-2-hydroxy-6-methyl-5-[(2-nitrophenyl)thio]benzoic acid,
3-tert-butyl-2-hydroxy-6-methyl-5-[(3-nitrophenyl)sulfonyl]benzoic acid,
3-tert-butyl-2-hydroxy-6-methyl-5-[(3-nitrophenyl)thio]benzoic acid,
3-tert-butyl-2-hydroxy-6-methyl-5-[(4-methylphenyl)sulfonyl]benzoic acid,
3-tert-butyl-2-hydroxy-6-methyl-5-[(4-methylphenyl)thio]benzoic acid,
3-tert-butyl-2-hydroxy-6-methyl-5-[(4-nitrophenyl)sulfinyl]benzoic acid,
3-tert-butyl-2-hydroxy-6-methyl-5-[(4-nitrophenyl)sulfonyl]benzoic acid,
3-tert-butyl-2-hydroxy-6-methyl-5-[(4-nitrophenyl)thio]benzoic acid,
3-tert-butyl-5-[(2,4-dinitrophenyl)sulfinyl]-2-hydroxy-6-methylbenzoic acid,
3-tert-butyl-5-[(2,4-dinitrophenyl)sulfonyl]-2-hydroxy-6-methylbenzoic acid,
3-tert-butyl-5-[(2,4-dinitrophenyl)thio]-2-hydroxy-6-methylbenzoic acid,
3-tert-butyl-5-[(2,5-dichlorophenyl)sulfonyl]-2-hydroxy-6-methylbenzoic acid,
3-tert-butyl-5-[(2,5-dichlorophenyl)thio]-2-hydroxy-6-methylbenzoic acid,
3-tert-butyl-5-[(2-chloro-5-nitrophenyl)sulfinyl]-2-hydroxy-6-methylbenzoic acid,
3-tert-butyl-5-[(2-chloro-5-nitrophenyl)sulfonyl]-2-hydroxy-6-methylbenzoic acid,
3-tert-butyl-5-[(2-chloro-5-nitrophenyl)thio]-2-hydroxy-6-methylbenzoic acid,
3-tert-butyl-5-[(3,4-dichlorophenyl)sulfonyl]-2-hydroxy-6-methylbenzoic acid,
3-tert-butyl-5-[(3,4-dichlorophenyl)thio]-2-hydroxy-6-methylbenzoic acid,
3-tert-butyl-5-[(4-chloro-2-nitrophenyl)sulfonyl]-2-hydroxy-6-methylbenzoic acid,
3-tert-butyl-5-[(4-chloro-2-nitrophenyl)thio]-2-hydroxy-6-methylbenzoic acid,
3-tert-butyl-5-[(4-chloro-3-nitrophenyl)sulfonyl]-2-hydroxy-6-methylbenzoic acid,
3-tert-butyl-5-[(4-chloro-3-nitrophenyl)thio]-2-hydroxy-6-methylbenzoic acid,
3-tert-butyl-5-[(4-chlorophenyl)sulfonyl]-2-hydroxy-6-methylbenzoic acid,
3-tert-butyl-5-[(4-chlorophenyl)thio]-2-hydroxy-6-methylbenzoic acid,
5-[(4-bromophenyl)sulfonyl]-2-hydroxy-3-methylbenzoic acid,
5-[(4-chlorophenyl)sulfonyl]-2-hydroxy-3-methylbenzoic acid, or
5-[(4-chlorophenyl)thio]-2-hydroxy-3-methylbenzoic acid,
   which are disclosed by Brown et al in Journal of the Chemical Society, Perkin Transactions 1: Organic and Bio-Organic Chemistry 1978, (6), 633-8, as intermediates for the production of salicylanilides, which in turn are said to be used as pesticides and antiparasitic agents;
   3-tert-butyl-5-(4-chlorobenzoyl)-2-hydroxy-6-methylbenzoic acid, which is disclosed by Brown & al in Journal of Medicinal Chemistry 1985, 28(1), 143-6, as an intermediate for the production of salicylanilides, which in turn are tested as flukicides;
   3-bromo-5-tert-butyl-6-hydroxy-2-methylbenzoic acid, which is disclosed in WO 2004/041256 as starting substance for production of chemical uncouplers intended for use of in treating obesity and obesity related diseases and conditions, or 3-tert-butyl-2-hydroxy-5-iodo-6-methylbenzoic acid, which is disclosed in U.S. Pat. No. 4,005,218 as starting substance for production of salicylanilide derivatives showing parasiticidal activity.

In another aspect of the invention there is provided the compound of formula I, or pharmaceutically acceptable salts thereof,

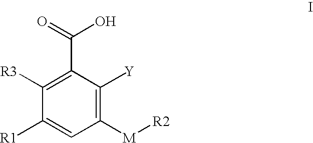

wherein
Y is selected from hydrogen, —OH, halo, —OC₁₋₆alkyl, and —C₁₋₆alkyl, the two latter optionally substituted with halo, —CN, —OH, —CF₃, —NH₂;
R1 is selected from —C₃₋₆cycloalkyl, heterocycloalkyl, aryl, alkylaryl, heteroaryl, and —C₃₋₆alkyl, optionally substituted with halo, —CN, —OH, —CF₃, —OCF₃, —NH₂, —CONH₂;
M is selected from —C(O)—, —C(H₂)—, —CH(OR^{a})—, —N(OH)—, —N(R^{a})—, —S(O)ᵣ—, heteroaryl and a bond; wherein R^{a} is hydrogen or C₁₋₆alkyl and r is 0, 1 or 2;
R2 is either selected from hydrogen, halo, —CN, or is a group D selected from —C₁₋₆alkyl, C₃₋₆cycloalkyl, heterocycloalkyl, —N(CH₃)₂, aryl, alkylaryl, heteroaryl, and heterocyclic groups,
where D is optionally substituted with one or more substituents G selected from halo, —NO₂, —CN, —OH, —CF₃, —OCF₃, —NH₂, —CONH₂, —COOH, aryl, heteroaryl, heterocyclic groups, —C₁₋₆alkyl, —C₁₋₆alkoxy, heterocycloalkyl, and C₁₋₆alkylcarboxylate;
where D may optionally be connected to G by a linker group L selected from —C(O)—, —S—, and —S(O₂)—;
and G, if substitutable, is optionally further substituted with one or more substituents selected from halo, —NO₂, —CN, —OH, —CH₃, —OCH₃, —CF₃, —OCF₃, —NH₂, —CONH₂, —COOH, and C₁₋₆alkylcarboxylate;
and R3 is selected from —OH and C₁₋₆alkoxy;
provided that when M is a bond and R3 is —OH, then R2 is not —C₁₋₆alkyl,
for use in therapy.

In a further aspect the present invention relates to compounds according to Formula I for the treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; opthalmological disorders such as retinopathies, diabetic retinopathies or glaucoma; psychiatric disorders, such as alcoholism, drug addiction and psychosis; inflammation related diseases, such as rheumatoid arthritis and osteoarthritis; and/or arthrosclerosis and stroke.

In a further aspect of the invention there is provided a pharmaceutical composition comprising a therapeutically effective amount of the compound of formula I in association with one or more pharmaceutically acceptable diluent, excipients and/or inert carrier, especially for the treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and postoperative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; opthalmological disorders such as retinopathies, diabetic retinopathies or glaucoma; psychiatric disorders, such as alcoholism, drug addiction and psychosis; inflammation related diseases, such as rheumatoid arthritis and osteoarthritis; and/or arthrosclerosis and stroke.

In a further aspect of the invention there is provided a pharmaceutical composition comprising a therapeutically effective amount of the compound of formula I in association with one or more pharmaceutically acceptable diluent, excipients and/or inert carrier, especially for the treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; opthalmological disorders such as retinopathies, diabetic retinopathies or glaucoma; psychiatric disorders, such as alcoholism, drug addiction and psychosis; inflammation related diseases, such as rheumatoid arthritis and osteoarthritis; and/or arthrosclerosis and stroke.

Another aspect of the invention relates to the use of the compound according formula I in the manufacture of a medicament for the treatment of neuropathic or inflammatory pain syndromes, such as arthritis, ischemia, cancer, fibromyalgia, low back pain and post-operative pain; migraine and tinnitus; inflammation related diseases, such as rheumatoid arthritis, osteoarthritis, and pneumological diseases; and arthrosclerosis and stroke.

In a further aspect of the invention there is provided a method of treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; opthalmological disorders such as retinopathies, diabetic retinopathies or glaucoma; psychiatric disorders, such as alcoholism, drug addiction and psychosis; inflammation related diseases, such as rheumatoid arthritis and osteoarthritis; and/or arthrosclerosis and stroke, comprising administering to a mammal, including man, in need of such treatment, a therapeutically effective amount of the compound according formula I.

In yet another aspect of the invention there is provided processes for the preparation of compounds of formula I.

These and other aspects of the present invention are described in greater detail herein below.

### DETAILED DESCRIPTION OF THE INVENTION

Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the other definitions for that group.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “Cₘ₋ₙ” or “Cₘ₋ₙ group” used alone or as a prefix, refers to any group having m to n carbon atoms.

For the avoidance of doubt it is to be understood that in this specification ‘C₁₋₆’ means a carbon group having 1, 2, 3, 4, 5 or 6 carbon atoms.

In the case where a subscript is the integer 0 (zero) the group to which the subscript refers to indicates that the group is absent.

In this specification unless otherwise stated the term “heteroatom” refers to an atom which is not carbon or hydrogen. Examples of heteroatoms include but are not limited to nitrogen, oxygen, and sulfur.

In this specification, unless stated otherwise, the term “alkyl” includes both straight and branched chain alkyl groups. The term “C₁₋₆alkyl” means an alkyl group having 1 to 6 carbon atoms and may be, but is not limited to, methyl, ethyl, n-propyl, i-propyl, n-butyl, i-butyl, s-butyl, t-butyl, n-pentyl, i-pentyl, t-pentyl, neo-pentyl, n-hexyl, i-hexyl, or t-hexyl. Similarly, the term “C₃₋₆alkyl” means an alkyl group having 3 to 6 carbon atoms and may be, but is not limited to, n-propyl, i-propyl, n-butyl, i-butyl, s-butyl, t-butyl, n-pentyl, i-pentyl, t-pentyl, neo-pentyl, n-hexyl, i-hexyl, or t-hexyl; and the term “C₃₋₄alkyl” means an alkyl group having 3 to 4 carbon atoms and may be, but is not limited to, n-propyl, i-propyl, n-butyl, i-butyl, s-butyl, or t-butyl.

In this specification, unless stated otherwise, the term “alkoxy” includes both straight or branched alkoxy groups. C₁₋₆alkoxy may be, but is not limited to, methoxy, ethoxy, n-propoxy, i-propoxy, n-butoxy, i-butoxy, s-butoxy, t-butoxy, n-pentoxy, i-pentoxy, t-pentoxy, neo-pentoxy, n-hexoxy, i-hexoxy, or t-hexoxy.

In this specification, unless stated otherwise, the term “halo” and “halogen” may be fluoro, chloro, bromo, or iodo.

In this specification, unless stated otherwise, the term “aryl” includes both aromatic monocyclic and bicyclic systems containing from 5 to 10 carbon atoms; in the case of a bicyclic system, at least one of the rings is of aromatic character, while the other ring may be aromatic or partially hydrogenated. Non-limiting examples of the term “aryl” are phenyl, naphthyl, indenyl, and tetralinyl.

In this specification, unless stated otherwise, the term “alkylaryl” means an aryl group having one or more alkyl groups pendant therefrom. Non-limiting examples of the term “alkylaryl” are benzyl, ethylnaphthyl, propylindenyl, and butyltetralinyl.

In this specification, unless stated otherwise, the term “heteroaryl” includes aryl groups as described above in which 1 to 4 carbon atoms are replaced by 1 to 4 hetero atoms, identical or different, selected independently of each other from oxygen, sulfur and nitrogen. Non-limiting examples of the term “heteroaryl” are furyl, imidazolyl, isoxazolyl, isothiazolyl, oxazolyl, pyrazinyl, pyrazolyl, pyridazinyl, pyridyl, pyrimidyl, pyrrolyl, thiazolyl or thienyl.

In this specification, unless stated otherwise, the term “cycloalkyl” includes both monocyclic and polycyclic systems containing from 3 to 10 carbon atoms, the systems being saturated or partially unsaturated but without aromatic character and it being understood that in the case of a polycyclic system one or more of the cycle(s) could be fused together or form a link. By the term “C₃₋₆cycloalkyl” is meant a cycloalkyl group containing from 3 to 6 carbon atoms, and may be, but is not limited to, cyclopropyl, cyclobutyl, cyclopentyl, or cyclohexyl.

In this specification, unless stated otherwise, a “heterocyclic group” is a an aromatic, partially aromatic, non-aromatic, saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)— and a ring sulphur atom may be optionally oxidised to form the S-oxide(s). Non-limiting examples of the term “heterocyclic group” are morpholino, piperidyl, pyridyl, pyranyl, pyrrolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, 4-pyridone, 1-isoquinolone, 2-pyrrolidone and 4-thiazolidone.

In this specification, unless stated otherwise, the term “heterocycloalkyl” includes cycloalkyl groups as defined hereinbefore in which 1 to 4 carbon atoms are replaced by 1 to 4 heteroatoms. Non-limiting examples of the term “heterocycloalkyl” are tetrahydrofuran, tetrahydrothiophene, piperidine, piperazine, morpholine, thiomorpholine, tetrahydropyran, tetrahydrothiopyran.

In this specification, unless stated otherwise, the term “alkylcarboxylate” is an alkyl that possesses a carboxyl group in any position. The term “C₁₋₆alkylcarboxylate” means a group R′C(O)O— or —C(O)OR′ where R′ is an alkyl group having 1 to 6 carbon atoms and may be, but is not limited to, methylcarboxylate, ethylcarboxylate, n-propylcarboxylate, i-propylcarboxylate, n-butylcarboxylate, i-butylcarboxylate, s-butylcarboxylate, t-butylcarboxylate, n-pentylcarboxylate, i-pentylcarboxylate, t-pentylcarboxylate, neo-pentylcarboxylate, n-hexylcarboxylate, i-hexylcarboxylate, or t-hexylcarboxylate.

One aspect of the invention relates to compounds of formula I, wherein Y may be independently selected from hydrogen, —OH, —OC₁₋₆alkyl, and —C₁₋₆alkyl. In specific aspect Y may be independently selected from hydrogen, —OH, —CH₃, and —OCH₃. In more specific aspect Y may be independently selected from —OH, —CH₃, and —OCH₃.

According to one aspect of the invention R1 may be independently selected from aryl, heteroaryl, —C₃₋₆cycloalkyl and —C₃₋₄-alkyl. In a specific aspect R1 may be independently selected from phenyl, pyridyl, —C₃₋₄-alkyl and cyclohexyl.

According to one aspect of the invention R1 may be independently selected from —C₃₋₆cycloalkyl and —C₃₋₄alkyl. In a specific aspect R1 may be independently selected from —C₃₋₄-alkyl and cyclohexyl.

According to one aspect of the invention, M may be independently selected from —C(O)—, —C(H₂)—, —CH(OC₂H₅)—, —S(O)₂—, —S—, —N(OH)—, —N(H)—, —N(CH₃)—, oxadiazolyl, and a bond.

According to one aspect of the invention, R2 may be independently selected from hydrogen, halo, and —CN.

According to another aspect of the invention R2 is a group D selected from phenyl, cyclohexyl, pyridinyl, benzyl, thiazolyl, naphthyl, —N(CH₃)₂, quinoxalinyl, —CN, oxypyridinyl, —CH₃, t-butyl, propyl, thiophenyl, and dioxido-benzothienyl.

According to one aspect of the invention G may be independently selected from —NH₂, —CONH₂, —Br, —Cl, —CN, —F, —OH, —I, —OCH₃, —NO₂, t-butyl, —COOH, —COOCH₃, —OCF₃, isopropyl, phenyl, —CH₃, —C₂H₅, morpholinyl, pyridinyl, benzothiazolyl, and —CF₃.

According to one aspect of the invention R3 may be —OH or —OCH₃.

According to one aspect of the invention,

Y is selected from hydrogen, —OH, —CH₃, and —OCH₃;
R1 is selected from phenyl, pyridyl, —C₃₋₄-alkyl and cyclohexyl;
M is selected from —C(O)—, —C(H₂)—, —CH(OC₂H₅)—, —S(O)₂—, —S—, —N(OH)—, —N(H)—, —N(CH₃)—, oxadiazolyl, and a bond;
R2 is selected from hydrogen, halo, and —CN;
D is selected from phenyl, cyclohexyl, pyridinyl, benzyl, thiazolyl, naphthyl, —N(CH₃)₂, quinoxalinyl, —CN, oxypyridinyl, —CH₃, t-butyl, propyl, thiophenyl, and dioxido-benzothienyl;
G is selected from —NH₂, —CONH₂, —Br, —Cl, —CN, —F, —OH, —I, —OCH₃, —NO₂, t-butyl, —COOH, —COOCH₃, —OCF₃, isopropyl, phenyl, —CH₃, —C₂H₅, morpholinyl, pyridinyl, benzothiazolyl, and

### —CF₃; and

### R3 is —OH or —OCH₃.

According to one aspect of the invention relates to a compound, which is selected from the group consisting of
3-tert-butyl-5-(4-chloro-3-iodobenzoyl)-2-hydroxy-6-methylbenzoic acid,
3-tert-Butyl-5-(4-tert-Butyl-benzoyl)-2-hydroxy-6-methyl-benzoic acid,
3-tert-Butyl-5-(4-trifluoromethoxy-benzoyl)-2-hydroxy-6-methyl-benzoic acid,
3-benzoyl-5-tert-butyl-6-hydroxy-2-methylbenzoic acid,
3-tert-butyl-5-(4-chloro-2-fluorobenzoyl)-2-hydroxy-6-methylbenzoic acid,
3-tert-butyl-5-(4-chloro-3-fluorobenzoyl)-2-hydroxy-6-methylbenzoic acid,
3-tert-Butyl-2,6-dihydroxy-benzoic acid,
3-tert-Butyl-5-(4-chloro-benzoyl)-2,6-dihydroxy-benzoic acid,
3-tert-butyl-5-(3,4-difluoro-benzoyl)-2,6-dihydroxy-benzoic acid,
3-tert-butyl-2,6-dihydroxy-5-(quinoxalin-2-ylcarbonyl)benzoic acid,
3-(4-chloro-benzoyl)-5-cyclohexyl-2,6-dihydroxy-benzoic acid,
3-tert-Butyl-5-[(4-chloro-phenyl)-hydroxyimino-methyl]-2-hydroxy-6-methyl-benzoic acid,
5,5′-di-tert-butyl-4,4′-dihydroxy-3′-(methoxycarbonyl)-2,2′-dimethylbiphenyl-3-carboxylic acid,
3-tert-Butyl-5-(4-fluorobenzoyl)-2-hydroxy-6-methylbenzoic acid,
3-tert-butyl-2-hydroxy-6-methyl-5-(4-methylbenzoyl)benzoic acid,
3-tert-butyl-5-(3,4-dichlorobenzoyl)-2-hydroxy-6-methylbenzoic acid,
3-tert-butyl-2-hydroxy-6-methyl-5-[4-(trifluoromethyl)benzoyl]benzoic acid,
3-tert-butyl-5-(2,4-dichlorobenzoyl)-2-hydroxy-6-methylbenzoic acid,
3-tert-butyl-2-hydroxy-6-methyl-5-[3-(trifluoromethoxy)benzoyl]benzoic acid,
3-tert-butyl-2-hydroxy-5-(3-isopropylbenzoyl)-6-methylbenzoic acid,
3-tert-butyl-2-hydroxy-6-methyl-5-(3-nitrobenzoyl)benzoic acid,
3-tert-butyl-2-hydroxy-5-(2-hydroxybenzoyl)-6-methylbenzoic acid,
3-tert-butyl-2-hydroxy-6-methyl-5-[2-(trifluoromethyl)benzoyl]benzoic acid,
5-tert-butyl-4-hydroxy-2-methylbiphenyl-3-carboxylic acid,
5-tert-butyl-4-hydroxy-2,2′-dimethylbiphenyl-3-carboxylic acid,
5-tert-butyl-4-hydroxy-4′-methoxy-2,2′-dimethylbiphenyl-3-carboxylic acid,
5-tert-butyl-4-hydroxy-2,2′-dimethylbiphenyl-3-carboxylic acid,
5-tert-butyl-4-hydroxy-4′-methoxy-2-methylbiphenyl-3-carboxylic acid,
5-tert-butyl-4-hydroxy-3′-isopropyl-2-methylbiphenyl-3-carboxylic acid,
3′,5-di-tert-butyl-4-hydroxy-2,5′-dimethylbiphenyl-3-carboxylic acid,
3-anilino-5-tert-butyl-6-hydroxy-2-methylbenzoic acid,
3-tert-butyl-5-[(4-chlorophenyl)amino]-2-hydroxy-6-methylbenzoic acid,
3-tert-butyl-5-[(4-chlorophenyl)(methyl)amino]-2-hydroxy-6-methylbenzoic acid,
3-tert-Butyl-5-[5-(4-chlorophenyl)-[1,2,4]oxadiazol-3-yl]-2-hydroxy-6-methylbenzoic acid,
3-tert-butyl-2-hydroxy-5-[(4-methoxyphenyl)thio]-6-methylbenzoic acid,
3-tert-butyl-2-hydroxy-6-methyl-5-(1-naphthylthio)benzoic acid,
3-[(2,4-dichlorophenyl)thio]-6-hydroxy-5-isopropyl-2-methylbenzoic acid,
3-tert-butyl-5-[(2,4-dichlorophenyl)thio]-2,6-dihydroxybenzoic acid,
2-hydroxy-3-isopropyl-6-methyl-5-(1-naphthylthio)benzoic acid,
3-tert-butyl-2-hydroxy-6-methyl-5-[(4-phenyl-1,3-thiazol-2-yl)thio]benzoic acid,
3-tert-butyl-2,6-dihydroxy-5-(1-naphthylthio)benzoic acid,
3-tert-butyl-5-[(2,4-dichlorophenyl)thio]-2-hydroxy-6-methylbenzoic acid,
3-(benzylthio)-5-tert-butyl-6-hydroxy-2-methylbenzoic acid,
3-tert-butyl-5-[(2,3-difluorobenzyl)thio]-2-hydroxy-6-methylbenzoic acid,
3-tert-butyl-5-[(4-chlorobenzyl)thio]-2-hydroxy-6-methylbenzoic acid,
3-(benzylsulfinyl)-5-tert-butyl-6-hydroxy-2-methylbenzoic acid,
3-(benzylsulfonyl)-5-tert-butyl-6-hydroxy-2-methylbenzoic acid,
3-tert-butyl-2-hydroxy-5-[(4-methoxyphenyl)sulfonyl]-6-methylbenzoic acid,
3-tert-butyl-2-hydroxy-6-methyl-5-(1-naphthylsulfonyl)benzoic acid,
3-tert-butyl-5-[(2,4-dichlorophenyl)sulfonyl]-2,6-dihydroxybenzoic acid,
3-[(2,4-dichlorophenyl)sulfonyl]-6-hydroxy-5-isopropyl-2-methylbenzoic acid,
3-tert-butyl-5-[(2,4-dichlorophenyl)sulfonyl]-2-hydroxy-6-methylbenzoic acid,
3-tert-butyl-5-[(4-chlorophenyl)(ethoxy)methyl]-2-hydroxy-6-methylbenzoic acid,
3,5-di-tert-butyl-2,6-dimethoxybenzoic acid,
3-tert-butyl-5-[(2,3-difluorobenzyl)thio]-2-hydroxy-6-methylbenzoic acid,
3-tert-butyl-2-hydroxy-6-methyl-5-(pyridin-4-ylthio)benzoic acid,
2-hydroxy-3-isopropyl-6-methyl-5-(1-naphthylsulfonyl)benzoic acid,
3-tert-butyl-5-{[(5-fluoro-1,3-benzothiazol-2-yl)methyl]thio}-2-hydroxy-6-methylbenzoic acid,
3-tert-butyl-2-hydroxy-5-[(3-methoxybenzyl)thio]-6-methylbenzoic acid,
3-tert-butyl-5-[(2-cyanobenzyl)thio]-2-hydroxy-6-methylbenzoic acid,
3-tert-butyl-2-hydroxy-6-methyl-5-[(tetrahydro-2H-pyran-2-ylmethyl)thio]benzoic acid,
3-tert-butyl-2-hydroxy-6-methyl-5-[(pyridin-3-ylmethyl)thio]benzoic acid,
3-tert-butyl-2-hydroxy-6-methyl-5-[(pyridin-4-ylmethyl)thio]benzoic acid,
3-tert-butyl-2-hydroxy-5-(isobutylthio)-6-methylbenzoic acid,
3-tert-butyl-2-hydroxy-6-methyl-5-[(2-phenylethyl)thio]benzoic acid,
3-tert-butyl-2-hydroxy-6-methyl-5-{[2-(trifluoromethyl)benzyl]thio}-benzoic acid,
3-tert-butyl-5-[(2,3-difluorobenzyl)sulfonyl]-2-hydroxy-6-methylbenzoic acid,
3-tert-butyl-5-[(4-chlorobenzyl)sulfonyl]-2-hydroxy-6-methylbenzoic acid,
3-tert-butyl-2-hydroxy-6-methyl-5-[(pyridin-2-ylmethyl)sulfonyl]benzoic acid,
3-tert-butyl-2-hydroxy-6-methyl-5-[(3-methylbenzyl)sulfonyl]benzoic acid,
3-tert-butyl-2-hydroxy-6-methyl-5-[(3-methylbenzyl)thio]benzoic acid,
3-tert-butyl-2-hydroxy-6-methyl-5-{[2-(trifluoromethyl)benzyl]sulfonyl}-benzoic acid,
3-tert-butyl-2-hydroxy-6-methyl-5-(phenylacetyl)benzoic acid,
3-tert-butyl-2-hydroxy-6-methyl-5-[phenyl(phenylthio)acetyl]benzoic acid,
3,5-Di-tert-butyl-2-chloro-6-hydroxybenzoic acid,
3-tert-butyl-5-[(3,4-difluorophenyl)thio]-2-hydroxy-6-methylbenzoic acid,
3-tert-butyl-5-[(3,4-difluorophenyl)sulfonyl]-2-hydroxy-6-methylbenzoic acid,
3-tert-butyl-2-hydroxy-6-methyl-5-[(2,4,5-trichlorophenyl)sulfonyl]benzoic acid,
3-tert-butyl-2-hydroxy-6-methyl-5-{[4-(trifluoromethoxy)phenyl]sulfonyl}benzoic acid,
3-{[3,5-Bis(trifluoromethyl)phenyl]sulfonyl}-5-tert-butyl-6-hydroxy-2-methylbenzoic acid,
3-tert-butyl-5-[(2,6-dichlorophenyl)sulfonyl]-2-hydroxy-6-methylbenzoic acid,
3-tert-butyl-5-[(2,3-dichlorophenyl)sulfonyl]-2-hydroxy-6-methylbenzoic acid,
3-tert-butyl-5-[(2-chloro-4-fluorophenyl)sulfonyl]-2-hydroxy-6-methylbenzoic acid,
3-tert-butyl-5-[(3-chloro-4-fluorophenyl)sulfonyl]-2-hydroxy-6-methylbenzoic acid,
3-tert-butyl-5-[(3,5-dichlorophenyl)sulfonyl]-2-hydroxy-6-methylbenzoic acid,
3′-tert-butyl-4-hydroxy-5′-methyl-5-pyridin-3-yl)biphenyl-3-carboxylic acid,
3-(1-Benzofuran-2-yl)-5-tert-butyl-6-hydroxy-2-methylbenzoic acid,
3-tert-butyl-5-(1,1-dioxido-1-benzothien-2-yl)-2-hydroxy-6-methylbenzoic acid,
5-tert-butyl-3′,4′-dichloro-4-hydroxy-2-methylbiphenyl-3-carboxylic acid,
5-tert-butyl-2′,4′-dichloro-4-hydroxy-2-methylbiphenyl-3-carboxylic acid,
5-tert-butyl-4-hydroxy-2-methyl-4′-morpholin-4-yl)biphenyl-3-carboxylic acid,
3-tert-butyl-2-hydroxy-6-methyl-5-(1-naphthyl)benzoic acid,
5-tert-butyl-3′-cyano-4-hydroxy-2-methylbiphenyl-3-carboxylic acid,
5-tert-butyl-4-hydroxy-2-methyl-3′,5′-bis(trifluoromethyl)biphenyl-3-carboxylic acid,
3-tert-butyl-2-hydroxy-6-methyl-5-(2-naphthyl)benzoic acid,
3-tert-butyl-2-hydroxy-5-isoquinolin-4-yl-6-methylbenzoic acid,
3-tert-butyl-2-hydroxy-6-methyl-5-quinolin-3-ylbenzoic acid,
3-tert-butyl-2-hydroxy-6-methyl-5-quinolin-8-ylbenzoic acid,
3-tert-butyl-2-hydroxy-6-methyl-5-quinolin-6-ylbenzoic acid,
3-tert-butyl-2-hydroxy-6-methyl-5-quinolin-5-ylbenzoic acid,
4′-hydroxy-6′-methoxy-1,1′:3′,1″-terphenyl-5′-carboxylic acid,
4,4″-difluoro-4′-hydroxy-1,1′:3′,1″-terphenyl-5′-carboxylic acid,
3-tert-butyl-4′-hydroxy-5-methyl-1,1′:3′,1″-terphenyl-5′-carboxylic acid, and
2,6-dihydroxy-3,5-diisopropylbenzoic acid.

According to one aspect of the invention relates to a compound, which is selected from the group consisting of
2-hydroxy-3-isopropyl-6-methyl-5-[(4-nitrophenyl)sulfinyl]benzoic acid,
2-hydroxy-3-isopropyl-6-methyl-5-[(4-nitrophenyl)sulfonyl]benzoic acid,
2-hydroxy-3-isopropyl-6-methyl-5-[(4-nitrophenyl)thio]benzoic acid,
2-hydroxy-3-methyl-5-[(4-methylphenyl)sulfonyl]benzoic acid,
2-hydroxy-3-methyl-5-[(4-nitrophenyl)sulfinyl]benzoic acid,
2-hydroxy-3-methyl-5-[(4-nitrophenyl)sulfonyl]benzoic acid,
2-hydroxy-3-methyl-5-[(4-nitrophenyl)thio]benzoic acid,
3-[(4-bromo-3-methylphenyl)sulfonyl]-5-tert-butyl-6-hydroxy-2-methylbenzoic acid,
3-[(4-bromo-3-methylphenyl)thio]-5-tert-butyl-6-hydroxy-2-methylbenzoic acid,
3-[(4-bromophenyl)sulfonyl]-5-tert-butyl-6-hydroxy-2-methylbenzoic acid,
3-[(4-bromophenyl)thio]-5-tert-butyl-6-hydroxy-2-methylbenzoic acid,
3-[(4-chlorophenyl)sulfonyl]-6-hydroxy-5-isopropyl-2-methylbenzoic acid,
3-bromo-5-tert-butyl-6-hydroxy-2-methylbenzoic acid,
3-tert-butyl-2-hydroxy-5-[(4-nitrophenyl)sulfonyl]benzoic acid,
3-tert-butyl-2-hydroxy-5-[(4-nitrophenyl)thio]benzoic acid,
3-tert-butyl-2-hydroxy-6-methyl-5-(phenylsulfonyl)benzoic acid,
3-tert-butyl-2-hydroxy-6-methyl-5-(phenylthio)benzoic acid,
3-tert-butyl-2-hydroxy-6-methyl-5-[(2,4,5-trichlorophenyl)sulfinyl]benzoic acid,
3-tert-butyl-2-hydroxy-6-methyl-5-[(2,4,5-trichlorophenyl)sulfonyl]benzoic acid,
3-tert-butyl-2-hydroxy-6-methyl-5-[(2,4,5-trichlorophenyl)thio]benzoic acid,
3-tert-butyl-2-hydroxy-6-methyl-5-[(2-nitrophenyl)sulfonyl]benzoic acid,
3-tert-butyl-2-hydroxy-6-methyl-5-[(2-nitrophenyl)thio]benzoic acid,
3-tert-butyl-2-hydroxy-6-methyl-5-[(3-nitrophenyl)sulfonyl]benzoic acid,
3-tert-butyl-2-hydroxy-6-methyl-5-[(3-nitrophenyl)thio]benzoic acid,
3-tert-butyl-2-hydroxy-6-methyl-5-[(4-methylphenyl)sulfonyl]benzoic acid,
3-tert-butyl-2-hydroxy-6-methyl-5-[(4-methylphenyl)thio]benzoic acid,
3-tert-butyl-2-hydroxy-6-methyl-5-[(4-nitrophenyl)sulfinyl]benzoic acid,
3-tert-butyl-2-hydroxy-6-methyl-5-[(4-nitrophenyl)sulfonyl]benzoic acid,
3-tert-butyl-2-hydroxy-6-methyl-5-[(4-nitrophenyl)thio]benzoic acid,
3-tert-butyl-5-(4-chlorobenzoyl)-2-hydroxy-6-methylbenzoic acid,
3-tert-butyl-5-[(2,4-dinitrophenyl)sulfinyl]-2-hydroxy-6-methylbenzoic acid,
3-tert-butyl-5-[(2,4-dinitrophenyl)sulfonyl]-2-hydroxy-6-methylbenzoic acid,
3-tert-butyl-5-[(2,4-dinitrophenyl)thio]-2-hydroxy-6-methylbenzoic acid,
3-tert-butyl-5-[(2,5-dichlorophenyl)sulfonyl]-2-hydroxy-6-methylbenzoic acid,
3-tert-butyl-5-[(2,5-dichlorophenyl)thio]-2-hydroxy-6-methylbenzoic acid,
3-tert-butyl-5-[(2-chloro-5-nitrophenyl)sulfinyl]-2-hydroxy-6-methylbenzoic acid,
3-tert-butyl-5-[(2-chloro-5-nitrophenyl)sulfonyl]-2-hydroxy-6-methylbenzoic acid,
3-tert-butyl-5-[(2-chloro-5-nitrophenyl)thio]-2-hydroxy-6-methylbenzoic acid,
3-tert-butyl-5-[(3,4-dichlorophenyl)sulfonyl]-2-hydroxy-6-methylbenzoic acid,
3-tert-butyl-5-[(3,4-dichlorophenyl)thio]-2-hydroxy-6-methylbenzoic acid,
3-tert-butyl-5-[(4-chloro-2-nitrophenyl)sulfonyl]-2-hydroxy-6-methylbenzoic acid,
3-tert-butyl-5-[(4-chloro-2-nitrophenyl)thio]-2-hydroxy-6-methylbenzoic acid,
3-tert-butyl-5-[(4-chloro-3-nitrophenyl)sulfonyl]-2-hydroxy-6-methylbenzoic acid,
3-tert-butyl-5-[(4-chloro-3-nitrophenyl)thio]-2-hydroxy-6-methylbenzoic acid,
3-tert-butyl-5-[(4-chlorophenyl)sulfonyl]-2-hydroxy-6-methylbenzoic acid,
3-tert-butyl-5-[(4-chlorophenyl)thio]-2-hydroxy-6-methylbenzoic acid,
5-[(2,4-dinitrophenyl)sulfonyl]-2-hydroxy-3-methylbenzoic acid,
5-[(4-bromophenyl)sulfonyl]-2-hydroxy-3-methylbenzoic acid,
5-[(4-chlorophenyl)sulfonyl]-2-hydroxy-3-methylbenzoic acid,
5-[(4-chlorophenyl)thio]-2-hydroxy-3-methylbenzoic acid,
3-tert-butyl-2-hydroxy-5-iodo-6-methylbenzoic acid,
3-tert-butyl-5-[(2,3-difluorobenzyl)thio]-2-hydroxy-6-methylbenzoic acid,
3-tert-butyl-2-hydroxy-6-methyl-5-(pyridin-4-ylthio)benzoic acid,
2-hydroxy-3-isopropyl-6-methyl-5-(1-naphthylsulfonyl)benzoic acid,
3-tert-butyl-5-{[(5-fluoro-1,3-benzothiazol-2-yl)methyl]thio}-2-hydroxy-6-methylbenzoic acid,
3-tert-butyl-2-hydroxy-5-[(3-methoxybenzyl)thio]-6-methylbenzoic acid,
3-tert-butyl-5-[(2-cyanobenzyl)thio]-2-hydroxy-6-methylbenzoic acid,
3-tert-butyl-2-hydroxy-6-methyl-5-[(tetrahydro-2H-pyran-2-ylmethyl)thio]benzoic acid,
3-tert-butyl-2-hydroxy-6-methyl-5-[(pyridin-3-ylmethyl)thio]benzoic acid,
3-tert-butyl-2-hydroxy-6-methyl-5-[(pyridin-4-ylmethyl)thio]benzoic acid,
3-tert-butyl-2-hydroxy-5-(isobutylthio)-6-methylbenzoic acid,
3-tert-butyl-2-hydroxy-6-methyl-5-[(2-phenylethyl)thio]benzoic acid,
3-tert-butyl-2-hydroxy-6-methyl-5-{[2-(trifluoromethyl)benzyl]thio}-benzoic acid,
3-tert-butyl-5-[(2,3-difluorobenzyl)sulfonyl]-2-hydroxy-6-methylbenzoic acid,
3-tert-butyl-5-[(4-chlorobenzyl)sulfonyl]-2-hydroxy-6-methylbenzoic acid,
3-tert-butyl-2-hydroxy-6-methyl-5-[(pyridin-2-ylmethyl)sulfonyl]benzoic acid,
3-tert-butyl-2-hydroxy-6-methyl-5-[(3-methylbenzyl)sulfonyl]benzoic acid,
3-tert-butyl-2-hydroxy-6-methyl-5-[(3-methylbenzyl)thio]benzoic acid,
3-tert-butyl-2-hydroxy-6-methyl-5-{[2-(trifluoromethyl)benzyl]sulfonyl}-benzoic acid,
3-tert-butyl-2-hydroxy-6-methyl-5-(phenylacetyl)benzoic acid,
3-tert-butyl-2-hydroxy-6-methyl-5-[phenyl(phenylthio)acetyl]benzoic acid,
3,5-Di-tert-butyl-2-chloro-6-hydroxybenzoic acid,
3-tert-butyl-5-[(3,4-difluorophenyl)thio]-2-hydroxy-6-methylbenzoic acid,
3-tert-butyl-5-[(3,4-difluorophenyl)sulfonyl]-2-hydroxy-6-methylbenzoic acid,
3-tert-butyl-2-hydroxy-6-methyl-5-[(2,4,5-trichlorophenyl)sulfonyl]benzoic acid,
3-tert-butyl-2-hydroxy-6-methyl-5-{[4-(trifluoromethoxy)phenyl]sulfonyl}benzoic acid,
3-{[3,5-Bis(trifluoromethyl)phenyl]sulfonyl}-5-tert-butyl-6-hydroxy-2-methylbenzoic acid,
3-tert-butyl-5-[(2,6-dichlorophenyl)sulfonyl]-2-hydroxy-6-methylbenzoic acid,
3-tert-butyl-5-[(2,3-dichlorophenyl)sulfonyl]-2-hydroxy-6-methylbenzoic acid,
3-tert-butyl-5-[(2-chloro-4-fluorophenyl)sulfonyl]-2-hydroxy-6-methylbenzoic acid,
3-tert-butyl-5-[(3-chloro-4-fluorophenyl)sulfonyl]-2-hydroxy-6-methylbenzoic acid,
3-tert-butyl-5-[(3,5-dichlorophenyl)sulfonyl]-2-hydroxy-6-methylbenzoic acid,
3′-tert-butyl-4-hydroxy-5′-methyl-5-pyridin-3-yl)biphenyl-3-carboxylic acid,
3-(1-Benzofuran-2-yl)-5-tert-butyl-6-hydroxy-2-methylbenzoic acid,
3-tert-butyl-5-(1,1-dioxido-1-benzothien-2-yl)-2-hydroxy-6-methylbenzoic acid,
5-tert-butyl-3′,4′-dichloro-4-hydroxy-2-methylbiphenyl-3-carboxylic acid,
5-tert-butyl-2′,4′-dichloro-4-hydroxy-2-methylbiphenyl-3-carboxylic acid,
5-tert-butyl-4-hydroxy-2-methyl-4′-morpholin-4-yl)biphenyl-3-carboxylic acid,
3-tert-butyl-2-hydroxy-6-methyl-5-(1-naphthyl)benzoic acid,
5-tert-butyl-3′-cyano-4-hydroxy-2-methylbiphenyl-3-carboxylic acid,
5-tert-butyl-4-hydroxy-2-methyl-3′,5′-bis(trifluoromethyl)biphenyl-3-carboxylic acid,
3-tert-butyl-2-hydroxy-6-methyl-5-(2-naphthyl)benzoic acid,
3-tert-butyl-2-hydroxy-5-isoquinolin-4-yl-6-methylbenzoic acid,
3-tert-butyl-2-hydroxy-6-methyl-5-quinolin-3-ylbenzoic acid,
3-tert-butyl-2-hydroxy-6-methyl-5-quinolin-8-ylbenzoic acid,
3-tert-butyl-2-hydroxy-6-methyl-5-quinolin-6-ylbenzoic acid,
3-tert-butyl-2-hydroxy-6-methyl-5-quinolin-5-ylbenzoic acid,
4′-hydroxy-6′-methoxy-1,1′:3′,1″-terphenyl-5′-carboxylic acid,
4,4″-difluoro-4′-hydroxy-1,1′:3′,1″-terphenyl-5′-carboxylic acid,
3-tert-butyl-4′-hydroxy-5-methyl-1,1′:3′,1″-terphenyl-5′-carboxylic acid, and
2,6-dihydroxy-3,5-diisopropylbenzoic acid,
   for use in therapy.

A suitable pharmaceutically acceptable salt of the compound of the invention is, for example, an alkali metal salt, an alkaline earth metal salt or a salt with an organic base that affords a physiologically-acceptable cation.

Some compounds of formula I may have chiral centres and/or geometric isomeric centres (E- and Z-isomers), and it is to be understood that the invention encompasses all such optical, diastereoisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

### Pharmaceutical Composition

According to one aspect of the present invention there is provided a pharmaceutical composition comprising as active ingredient a therapeutically effective amount of the compound of formula I, or salts, solvates or solvated salts thereof, in association with one or more pharmaceutically acceptable diluent, excipients and/or inert carrier.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers.

Suitable daily doses of the compounds of formula I in the treatment of a mammal, including man, are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, severity of the illness being treated, the route of administration, the age, weight and sex of the patient and the particular compound being used, and may be determined by a physician.

### Medical Use

The compounds of the present invention are expected to be useful in the treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and postoperative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; opthalmological disorders such as retinopathies, diabetic retinopathies or glaucoma; psychiatric disorders, such as alcoholism, drug addiction and psychosis; inflammation related diseases, such as rheumatoid arthritis and osteoarthritis; and/or arthrosclerosis and stroke.

The invention relates to compounds of formula I as defined hereinbefore, for use in therapy; for the sake of clarity this also includes
2-hydroxy-3-isopropyl-6-methyl-5-[(4-nitrophenyl)sulfinyl]benzoic acid,
2-hydroxy-3-isopropyl-6-methyl-5-[(4-nitrophenyl)sulfonyl]benzoic acid,
2-hydroxy-3-isopropyl-6-methyl-5-[(4-nitrophenyl)thio]benzoic acid,
2-hydroxy-3-methyl-5-[(4-methylphenyl)sulfonyl]benzoic acid,
2-hydroxy-3-methyl-5-[(4-nitrophenyl)sulfinyl]benzoic acid,
2-hydroxy-3-methyl-5-[(4-nitrophenyl)sulfonyl]benzoic acid,
2-hydroxy-3-methyl-5-[(4-nitrophenyl)thio]benzoic acid,
3-[(4-bromo-3-methylphenyl)sulfonyl]-5-tert-butyl-6-hydroxy-2-methylbenzoic acid,
3-[(4-bromo-3-methylphenyl)thio]-5-tert-butyl-6-hydroxy-2-methylbenzoic acid,
3-[(4-bromophenyl)sulfonyl]-5-tert-butyl-6-hydroxy-2-methylbenzoic acid,
3-[(4-bromophenyl)thio]-5-tert-butyl-6-hydroxy-2-methylbenzoic acid,
3-[(4-chlorophenyl)sulfonyl]-6-hydroxy-5-isopropyl-2-methylbenzoic acid,
3-bromo-5-tert-butyl-6-hydroxy-2-methylbenzoic acid,
3-tert-butyl-2-hydroxy-5-[(4-nitrophenyl)sulfonyl]benzoic acid,
3-tert-butyl-2-hydroxy-5-[(4-nitrophenyl)thio]benzoic acid,
3-tert-butyl-2-hydroxy-6-methyl-5-(phenylsulfonyl)benzoic acid,
3-tert-butyl-2-hydroxy-6-methyl-5-(phenylthio)benzoic acid,
3-tert-butyl-2-hydroxy-6-methyl-5-[(2,4,5-trichlorophenyl)sulfonyl]benzoic acid,
3-tert-butyl-2-hydroxy-6-methyl-5-[(2,4,5-trichlorophenyl)sulfonyl]benzoic acid,
3-tert-butyl-2-hydroxy-6-methyl-5-[(2,4,5-trichlorophenyl)thio]benzoic acid,
3-tert-butyl-2-hydroxy-6-methyl-5-[(2-nitrophenyl)sulfonyl]benzoic acid,
3-tert-butyl-2-hydroxy-6-methyl-5-[(2-nitrophenyl)thio]benzoic acid,
3-tert-butyl-2-hydroxy-6-methyl-5-[(3-nitrophenyl)sulfonyl]benzoic acid,
3-tert-butyl-2-hydroxy-6-methyl-5-[(3-nitrophenyl)thio]benzoic acid,
3-tert-butyl-2-hydroxy-6-methyl-5-[(4-methylphenyl)sulfonyl]benzoic acid,
3-tert-butyl-2-hydroxy-6-methyl-5-[(4-methylphenyl)thio]benzoic acid,
3-tert-butyl-2-hydroxy-6-methyl-5-[(4-nitrophenyl)sulfinyl]benzoic acid,
3-tert-butyl-2-hydroxy-6-methyl-5-[(4-nitrophenyl)sulfonyl]benzoic acid,
3-tert-butyl-2-hydroxy-6-methyl-5-[(4-nitrophenyl)thio]benzoic acid,
3-tert-butyl-5-(4-chlorobenzoyl)-2-hydroxy-6-methylbenzoic acid,
3-tert-butyl-5-[(2,4-dinitrophenyl)sulfinyl]-2-hydroxy-6-methylbenzoic acid,
3-tert-butyl-5-[(2,4-dinitrophenyl)sulfonyl]-2-hydroxy-6-methylbenzoic acid,
3-tert-butyl-5-[(2,4-dinitrophenyl)thio]-2-hydroxy-6-methylbenzoic acid,
3-tert-butyl-5-[(2,5-dichlorophenyl)sulfonyl]-2-hydroxy-6-methylbenzoic acid,
3-tert-butyl-5-[(2,5-dichlorophenyl)thio]-2-hydroxy-6-methylbenzoic acid,
3-tert-butyl-5-[(2-chloro-5-nitrophenyl)sulfinyl]-2-hydroxy-6-methylbenzoic acid,
3-tert-butyl-5-[(2-chloro-5-nitrophenyl)sulfonyl]-2-hydroxy-6-methylbenzoic acid,
3-tert-butyl-5-[(2-chloro-5-nitrophenyl)thio]-2-hydroxy-6-methylbenzoic acid,
3-tert-butyl-5-[(3,4-dichlorophenyl)sulfonyl]-2-hydroxy-6-methylbenzoic acid,
3-tert-butyl-5-[(3,4-dichlorophenyl)thio]-2-hydroxy-6-methylbenzoic acid,
3-tert-butyl-5-[(4-chloro-2-nitrophenyl)sulfonyl]-2-hydroxy-6-methylbenzoic acid,
3-tert-butyl-5-[(4-chloro-2-nitrophenyl)thio]-2-hydroxy-6-methylbenzoic acid,
3-tert-butyl-5-[(4-chloro-3-nitrophenyl)sulfonyl]-2-hydroxy-6-methylbenzoic acid,
3-tert-butyl-5-[(4-chloro-3-nitrophenyl)thio]-2-hydroxy-6-methylbenzoic acid,
3-tert-butyl-5-[(4-chlorophenyl)sulfonyl]-2-hydroxy-6-methylbenzoic acid,
3-tert-butyl-5-[(4-chlorophenyl)thio]-2-hydroxy-6-methylbenzoic acid,
5-[(2,4-dinitrophenyl)sulfonyl]-2-hydroxy-3-methylbenzoic acid,
5-[(4-bromophenyl)sulfonyl]-2-hydroxy-3-methylbenzoic acid,
5-[(4-chlorophenyl)sulfonyl]-2-hydroxy-3-methylbenzoic acid,
5-[(4-chlorophenyl)thio]-2-hydroxy-3-methylbenzoic acid, and
3-tert-butyl-2-hydroxy-5-iodo-6-methylbenzoic acid.

The invention relates to compounds of formula I as defined hereinbefore, for use in treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and postoperative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; opthalmological disorders such as retinopathies, diabetic retinopathies or glaucoma; psychiatric disorders, such as alcoholism, drug addiction and psychosis; inflammation related diseases, such as rheumatoid arthritis and osteoarthritis; and/or arthrosclerosis and stroke.

The present invention specifically relates to compounds of formula I as defined hereinbefore, for use in treatment of neuropathic pain syndrome.

The present invention relates also to the use of a compound of formula I as defined hereinbefore, in the manufacture of a medicament for the treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; opthalmological disorders such as retinopathies, diabetic retinopathies or glaucoma; psychiatric disorders, such as alcoholism, drug addiction and psychosis; inflammation related diseases, such as rheumatoid arthritis and osteoarthritis; and/or arthrosclerosis and stroke.

One embodiment of the invention relates to the use of a compound according to formula I in the treatment of neuropathic pain syndrome.

Another embodiment of the invention relates to the use of a compound according to formula I, for the manufacture of a medicament for treatment of neuropathic pain syndrome.

The invention also provides a method of treatment of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus; opthalmological disorders such as retinopathies, diabetic retinopathies or glaucoma; psychiatric disorders, such as alcoholism, drug addiction and psychosis; inflammation related diseases, such as rheumatoid arthritis and osteoarthritis; and/or arthrosclerosis and stroke.

Specifically, the present invention provides a method of treatment of neuropathic pain syndrome.

The compounds according to the present invention may furthermore be used as as an analgesic, anticonvulsant, muscle-relaxant, anti-inflammatory agent, fertility enhancer, male contraceptive, or an antihypertensive agent.

The dose required for the therapeutic or preventive treatment of a particular disorder will necessarily be varied depending on the host treated, the route of administration and the severity of the illness being treated.

In the context of the present specification, the term “therapy” and “treatment” includes prevention and/or prophylaxis, unless there are specific indications to the contrary. The terms “therapeutic” and “therapeutically” should be construed accordingly.

### Non-Medical Use

In addition to their use in therapeutic medicine, the compounds of formula I, or salts, solvates or solvated salts thereof, are also useful as pharmacological tools in the development and standardisation of in vitro and in vivo test systems for the evaluation of the effects of neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain; pain associated with angina, renal or billiary colic, menstruation, migraine and gout, stroke, head trauma, anoxic and ischemic injuries, hypoglycaemia, cardiovascular diseases and/or cancer; auditory neuropathic disorders such as tinnitus;

opthalmological disorders such as retinopathies, diabetic retinopathies or glaucoma;
psychiatric disorders, such as alcoholism, drug addiction and psychosis;
inflammation related diseases, such as rheumatoid arthritis and osteoarthritis;
and/or arthrosclerosis and stroke.

### Methods of Preparation

Another aspect of the present invention provides processes for preparing compounds of formula I, or salts, solvates or solvated salts thereof. Processes for the preparation of the compounds in the present invention are described herein.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994). Techniques for purification of intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C.

### Preparation of End Products

A process for preparing a compound of formula I, wherein Y, R1, R2, and R3 are, unless specified otherwise, defined as in formula I, comprises of:

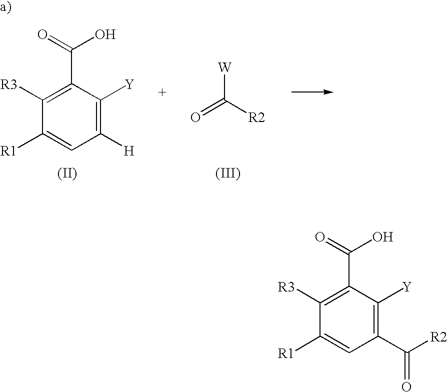

i) Reaction of an optionally protected compound of formula (II) with a compound of formula (III), wherein W is a halogen, such as Cl, Br or F, or a suitable leaving group, such as trifluoromethanesulfonyloxy, 4-toluenesulfonyloxy, alkylcarbonyloxy or hydroxy. The reaction is performed in a suitable solvent such as dichloromethane, dichloroethane, nitromethane, and advantageously in the presence of a Lewis acid such as AlCl₃, AlBr₃, Al(OR)₃, BF₃, BCl₃, BBr₃, ZnCl₂, FeCl₃, FeBr₃;
or

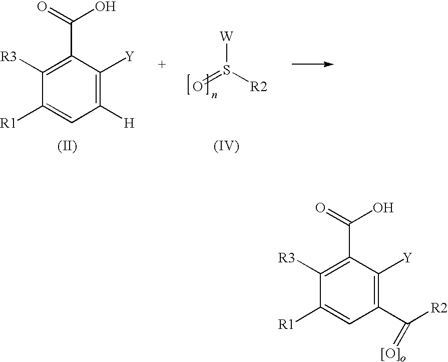

ii) Reaction of an optionally protected compound of formula (II) with a compound of formula (IV), wherein n is 0, 1 or 2, o is 0, 1 or 2, and W is a halogen such as Cl, Br or F. When n=0 or 1 the product from the first step can be oxidized by treatment with an oxidation reagent such as m-Chloroperbenzoic acid, hydrogen peroxide, NaIO₄, KMnO₄, PhICl₂ or t-BuOCl. The reaction is performed in a suitable solvent such as dichloromethane, dichloroethane, tetrahydrofuran, dimethylformamide, optionally in the presences of a Lewis acid such as AlCl₃, AlBr₃, Al(OR)₃, BF₃, BCl₃, BBr₃, ZnCl₂, FeCl₃, or FeBr₃, and when n=0, advantageously in the presence of a base such as pyridine, lutidine, triethylamine or Hünig's base at temperatures between −10° C. to reflux.

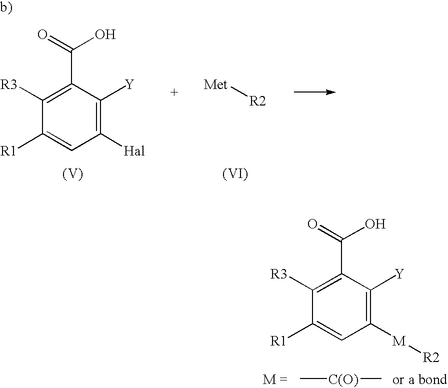

Reaction of an optionally protected compound of formula (V) with an organometallic reagent of formula (VI), wherein Hal is a halogen for example Br or I; or a sulfonyloxy group, for example methanesulfonyloxy, 4-toluenesulfonyloxy, or trifluoromethane-sulfonyloxy, and Met is a suitable metallic group, for example, copper, lithium, an organoboron reagent such as —B(OH)₂, —B(OPri)₂ or —B(Et)₂, in the presence of a carbon monoxide or dry nitrogen atmosphere, and in the presence of a metallic catalyst such as palladium or nickel, for example [1,1′-bis(diphenylphosphino)ferrocene]dichloro-palladium(II), tetrakis(triphenylphosphine)palladium(0), palladium(II) chloride, palladium(II) bromide, nickel(II)chloride, nickel(II) bromide or bis(triphenylphosphine)-nickel(II) chloride, and optionally in the presence of an additional ligand such as di-tert-butylphosphino pentaphenylferrocene or 2-dicyclohexylphosphino-2′,6′-dimethoxy-biphenyl in the presence of a suitable inert solvent or diluent, for example tetrahydrofuran, 1,4-dioxan, 1,2-dimethoxyethane, benzene, toluene, xylene, anisol, methanol or ethanol. When Hal is Br, potassium iodide can preferably be used as an additive. The reaction is preferably conducted in the presence of a suitable base such as, for example, sodium carbonate or potassium carbonate, potassium fluoride, potassium phosphate, pyridine,

4-dimethylaminopyridine, triethylamine or morpholine, and conveniently at a temperature in the range, for example 10 to 250° C., preferably in the range 60 to 120° C. Performing the reaction in the presence of carbon monoxide gives compounds where M is a carbonyl group, whereas performing the reaction in the absence of carbon monoxide gives compounds where M is a single bond.

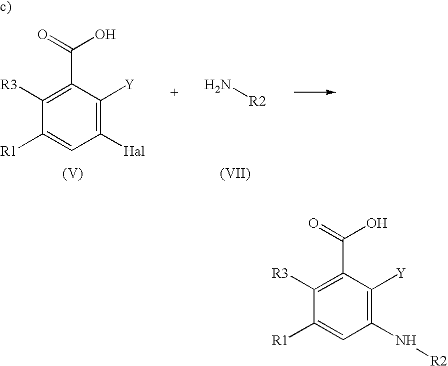

Reaction of an optionally protected halophenol of formula (V) with an amine of formula (VII), wherein Hal is a halogen for example Br or I; or a sulfonyloxy group, for example methanesulfonyloxy 4-toluenesulfonyloxy or trifluoromethanesulfonyloxy, and in the presence of a metallic catalyst such as palladium or nickel, for example bis(dibenzylideneacetone)platinum(0), [1,1′-bis(diphenylphosphino)ferrocene]dichloro-palladium(II), tetrakis(triphenylphosphine)-palladium(0), palladium(II) chloride, palladium(II) bromide, nickel(II) chloride, nickel(II) bromide or bis(triphenylphosphine)-nickel(II) chloride, and optionally in the presence of an additional ligand such as di-tert-butylphosphino pentaphenylferrocene or 2-dicyclohexylphosphino-2′,6′-dimethoxybiphenyl in the presence of a suitable inert solvent or diluent, for example tetrahydrofuran, 1,4-dioxan, 1,2-dimethoxyethane, benzene, toluene, xylene, anisol, methanol or ethanol.

When Hal is Br, potassium iodide can optionally be used as an additive.

The reaction is preferably conducted in the presence of a suitable base such as, for example, sodium carbonate or potassium carbonate, potassium fluoride, potassium phosphate, pyridine, 4-dimethylaminopyridine, triethylamine or morpholine, and conveniently at a temperature in the range, for example 10 to 250° C., preferably in the range 60 to 120° C.

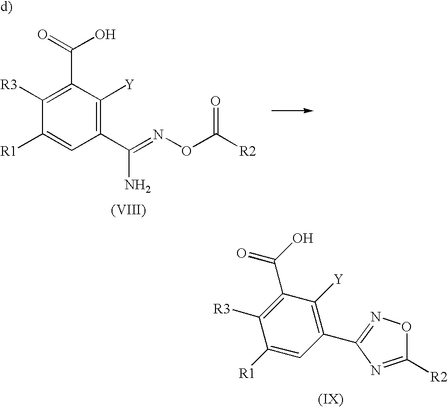

Reaction of an intermediate of formula (VIII), by heating in a suitable solvent for example dimethylformamide, dimethylacetamide, dimethylsulfoxide, N-methylpyrrolidone, N,N-dimethyl-propylene-urea, toluene, xylene, tetrachloroethane at temperatures between 30° C. and reflux, to give a compound of formula (IX).

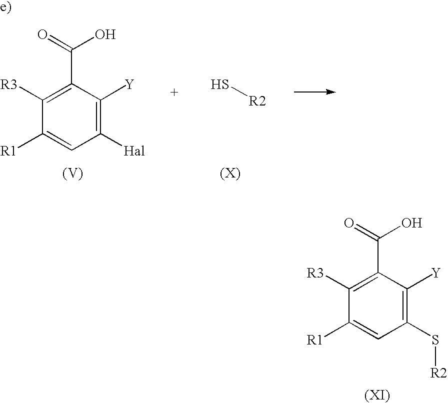

Reaction of an optionally protected halophenol of formula (V) with a mercaptan of formula (X), wherein Hal is a halogen for example Br or I; or a sulfonyloxy group, for example methanesulfonyloxy 4-toluenesulfonyloxy or trifluoromethanesulfonyloxy, in the presence of a metallic catalyst such as Cu(I) derivatives such as CuCl, CuBr, CuI, Cu(OCF₃) and in the presence of a suitable base such as an alkaline carbonate for example sodium carbonate, potassium carbonate or cesium carbonate in a suitable solvent or solvent mixture, for example mixtures of C₁₋₆diols and C₁₋₆alcohols such as ethylene or propylene glycol and 1-propanol, 2-propanol or tert-butanol by heating in an inert atmosphere at temperatures between 30° C. and reflux, to give a compound of formula (XI).

The product from the first step can then optionally be oxidized by treatment with an oxidation reagent such as m-Chloroperbenzoic acid, hydrogen peroxide, NaIO₄, KMnO₄, PhICl₂ or t-BuOCl to give a sulfoxide or sulfone.

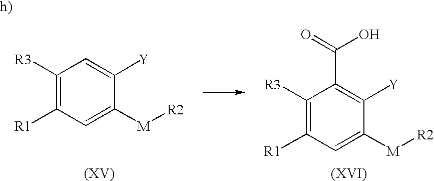

Reaction of an optionally protected cresol or resorcinol of formula (XV) with a suitable base such as n-Butyllithium, sodium metal, sodium-, potassium- or cesium carbonate, sodium-, potassium- or cesium hydrogen carbonate or sodium-, potassium- or cesium hydroxide and carbon dioxide, in a suitable solvent such as hexane, pentane, dimethylformamide, dimethylacetamide, N-methylpyrrolidone or pyridine at temperatures between −78° C. and reflux, optionally in an inert atmosphere to give a compound of formula (XVI).

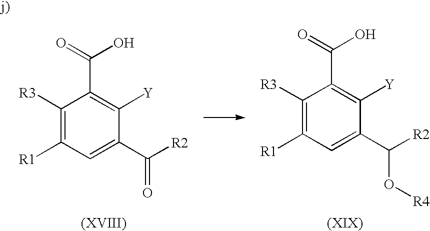

Reaction of an optionally protected compound of formula (XVIII) with a suitable reducing agent such as sodium borohydride, BH3-THF, borane-methylsulfide complex or by catalytic hydrogenation over a suitable catalyst for example palladium-on-carbon in a suitable solvent such as methanol, ethanol, tetrahydrofuran or ethyl acetate to give a compound of formula (XIX), where R4 are hydrogen or C₁₋₆alkyl.

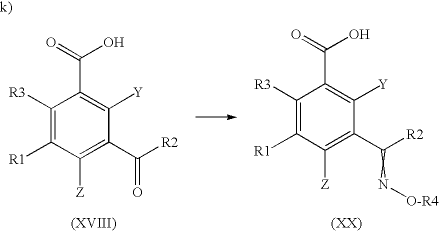

Reaction of an optionally protected compound of formula (XVIII) with optionally substituted hydroxylamine hydrochloride in the presence of a base such as triethylamine, sodium- or potassium carbonate or sodium- or potassium hydrogen carbonate in a solvent such as dichlorometane, 1,2-dichloroethane or toluene with removal of water at reflux temperature using for example a Dean-Stark trap to give a compound of formula (XX) where R4 are hydrogen, C₁₋₆alkyl or an optionally substituted aryl group.

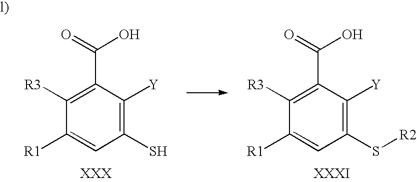

Reaction of an optionally protected compound of formula (XXX) with a suitable electrophile for example an halide or sulfonate in the presence of a suitable base for example triethylamine, Hünigs base, DBU, sodium or potassium carbonate or sodium or potassium hydrogencarbonate in a suitable solvent such as dimethylformamide at temperatures ranging from 0° C. to reflux to give a compound of formula XXXI.

### Preparation of Intermediates

Processes for the preparation of the intermediates required for preparation of the end products comprise:

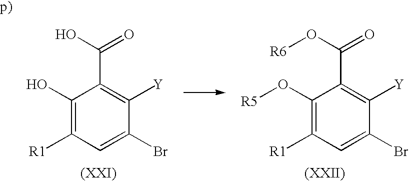

Reaction of an optionally protected compound of formula XXI with an alkylating agent such as methyl- or ethyl iodide, dimethyl sulfate or benzyl bromide in a suitable solvent such as dimethylformamide, dimethylsulfoxide or dichloromethane the presence of a base such as sodium-, potassium- or cesium carbonate or tritethylamine at temperatures between room temperature and reflux to give a compound such as XXII where R5 and R6 are suitable protecting groups such as ethyl, methyl or benzyl.

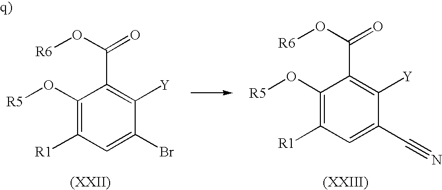

Reaction of an optionally protected compound of formula XXII with a suitable cyanide reagent such as CuCN, in a suitable solvent such as dimethylformamide, dimethylacetamide, N-methylpyrrolidone or dimethylsulfoxide at temperatures between 50° C. and reflux under an inert atmosphere to give a compound of formula XXIII, where R5 and R6 are suitable protecting groups such as ethyl, methyl or benzyl.

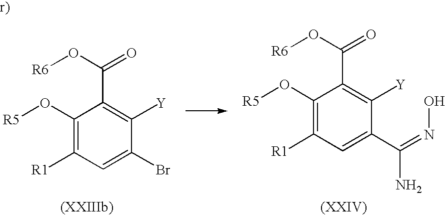

Reaction of an optionally protected compound of formula XXIIIb with hydroxylamine hydrochloride or a protected hydroxylamine hydrochloride derivative in the presence of a suitable base such as sodium- or potassium carbonate or triethylamine in a suitable solvent such as C₁₋₆alcohols, dimethylformamide, dimethylacetamide, N-methylpyrrolidone or dimethylsulfoxide at temperatures between ambient and reflux to give a compound of formula XXIV, where R5 and R6 are suitable protecting groups such as ethyl, methyl or benzyl.

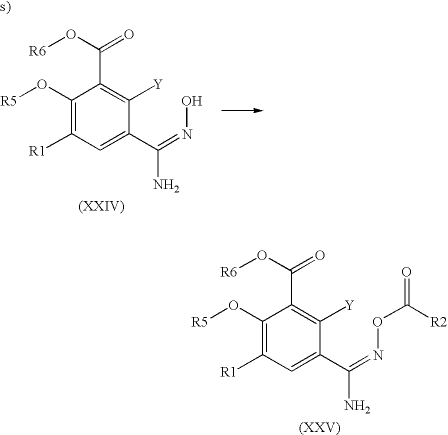

Reaction of an optionally protected compound of formula XXIV with a suitably activated carboxylic acid derivative such as an acid-fluoride, -chloride or -bromide, an activated ester or a mixed acid anhydride in the presence of suitable base such as Hünigs base, triethylamine or sodium-, potassium-, or cesium carbonate in a suitable solvent such as dimethylformamide, dimethylacetamide, N-methylpyrrolidone, dimethylsulfoxide, dichloromethane or dichloroethane at temperatures between −10° C. and reflux to give a compound of formula XXV, where R5 and R6 are suitable protecting groups such as ethyl, methyl or benzyl.

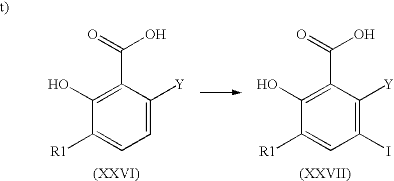

Reaction of an optionally protected compound of formula XXVI with a suitable iodination reagent such as iodine monochloride in a suitable solvent such as dimethylformamide, dimethylacetamide, N-methylpyrrolidone, dichloromethane or dichloroethane and in an inert atmosphere at temperatures between 0° C. and reflux to give a compound of formula XXVII.

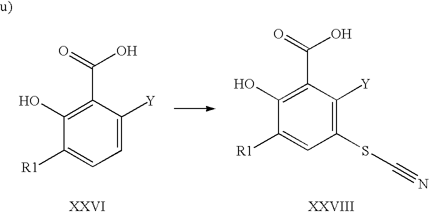

Reaction of an optionally protected compound of formula XXVI with suitable reagents for thiocyanation for example bromine and sodium or potassium thiocyanate in a suitable solvent such as methanol or ethanol at temperatures between −50° C. and reflux to give a compound of formula XXVIII.

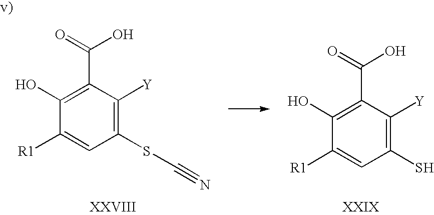

Reaction of an optionally protected compound of formula XXVIII with reducing agent, for example lithium aluminium hydride or sodium hydride in a suitable solvent such as diethyl ether or tetrahydrofuran. In another example the reducing agent is zinc in a solvent such as acetic acid and in yet another example the reduction is carried out by sodium sulfide or DL-thiothreitol, in a suitable solvent such as ethanol or water in the presence of a buffer such as KH₂PO₄ or NaH₂PO₄. The temperatures of the reactions range between −78° C. and reflux to give a compound of formula XXIX.

### EXAMPLES

The invention will now be illustrated by the following non-limiting examples. Unless otherwise indicated, all starting materials are commercially available or earlier described in the literature.

### End Products

### Example 1

### 3-tert-butyl-5-(4-chloro-3-iodobenzoyl)-2-hydroxy-6-methylbenzoic Acid

Anhydrous aluminium trichloride (0.270 g) was suspended in 3 ml dry 1,2-dichloroethane and a solution of 4-chloro-3-iodo-benzoyl chloride (0.650 g) in 3 ml dry 1,2-dichloroethane was added at ambient temperature under dry nitrogen. When all aluminium trichloride had dissolved the solution was cooled to −5° C. and a suspension of 3-tert-butyl-2-hydroxy-6-methyl-benzoic acid (0.208 g) in 3 ml dry 1,2-dichloroethane was added. The reaction mixture was stirred at −5 to 0° C. overnight. The reaction mixture was poured into 20 ml 1M HCl while stirring at 0° C. This mixture was extracted with 3×20 ml 1,2-dichloroethane, the extract was washed with water, dried over anhydrous sodium sulphate and then evaporated to give an oil (0.829 g). This material was triturated with petroleum ether to give a solid (0.222 g);

¹H NMR: (400 MHz, CHLOROFORM-d) δ ppm 1.40 (s, 9H) 2.46 (s, 3H) 7.36 (s, 1H) 7.54 (d, 1H) 7.68 (dd, 1H) 8.32 (d, 1H) 12.03 (s, 1H); Mass Spectrum: M−H⁺471.

The 4-chloro-3-iodo-benzoyl chloride used as a starting material was prepared as follows:

Thionyl chloride (10 ml) was added to 4-Chloro-3-iodo-benzoic acid (1.13 g) and the mixture was refluxed for 1 hour. The reaction mixture was evaporated to give a solid (1.20 g);

¹H NMR: (400 MHz, CHLOROFORM-d) δ ppm 7.59 (d, 1H) 8.03 (dd, 1H) 8.57 (d, 1H)

### Example 2-6

The following compounds were synthesised in an analogous method to Example 1

| Ex | Compound | ¹H NMR | m/z |
|---|---|---|---|
| 2 | 3-tert-Butyl-5-(4-tert-Butyl- | (400 MHz, CHLOROFORM-d) δ ppm | M-H+ |
| | benzoyl)-2-hydroxy-6-methyl- | 1.31-1.48 (m, 18 H) 2.46 (s, 3 H) | 367 |
| | benzoic acid | 7.37 (s, 1 H) 7.50 (t, 2 H) 7.77 (d, 1 H) | |
| | | 8.05 (d, 1 H) 11.93 (s, 1 H) | |
| 3 | 3-tert-Butyl-5-(4-trifluoro- | (400 MHz, METHANOL-d₄) δ ppm | M-H+ |
| | methoxy-benzoyl)-2-hydroxy- | 1.37 (s, 9 H) 2.44 (s, 3 H) 7.17 (s, 1 H) | 395 |
| | 6-methyl-benzoic acid | 7.39 (dd, 2 H) 7.86 (d, 2 H) | |
| 4 | 3-tert-Butyl-5-benzoyl-2-hy- | (400 MHz, METHANOL-d₄) δ ppm | M-H+ |
| | droxy-6-methyl-benzoic acid | 1.36 (s, 9 H) 2.44 (s, 3 H) 7.16 (s, 1 H) | 311 |
| | | 7.45-7.52 (m, 2 H) 7.57-7.64 (m, 1 | |
| | | H) 7.70-7.78 (m, 2 H) | |
| 5 | 3-tert-butyl-5-(4-chloro-2- | (400 MHz, DMSO-d₆) δ ppm | M-H+ |
| | fluorobenzoyl)-2-hydroxy-6- | 1.29 (s, 9 H) 2.40 (s, 3 H) 7.30 (s, 1 H), | 363 |
| | methylbenzoic acid | 7.45(dd, 1H) 7.59-7.63(m, 2H) | |
| 6 | 3-tert-butyl-5-(4-chloro-3- | (400 MHz, DMSO-d₆) δ ppm | M-H+ |
| | fluorobenzoyl)-2-hydroxy-6- | 1.33 (s, 9 H) 2.29 (s, 3 H) 7.26 (s, 1 H), | 363 |
| | methylbenzoic acid | 7.48(dd, 1H), 7.69(dd, 1H), 7.74- | |
| | | 7.7863(m, 1H) | |

### Example 7

### 3-tert-Butyl-2,6-dihydroxy-benzoic Acid

4-tert-Butyl-1,3-resorcinol (J. Org. Chem., 2001, 1935; 3.4 g) and potassium hydrogen carbonate (16.1 g) were added to dry dimethylformamide (150 ml) in a flask equipped with a distillation head. A continuous stream of CO₂ gas was passed into the flask via a pasteur pipette terminating above the liquid surface. The mixture was heated to 150° C. with stirring and reflux condensation overnight. Remaining dimethylformamide was removed via the distillation head at 150° C. and heating of the remaining melt was continued under a steady steam of CO₂ gas for 4 hours. The solid reaction mixture was allowed to stand at ambient temperature overnight and then partitioned between ethyl acetate (100 ml) and water (100 ml). Phases were separated and water (30 ml) was added to ethyl acetate phase and this mixture was then acidified to pH 2 using 6M HCl. The ethyl acetate phase was dried and evaporated to give crystalline product (4.07 g)

¹H NMR: (400 MHz, METHANOL-d₄) δ ppm 1.34 (s, 9H) 6.32 (d, 1H) 7.29 (d, 1H)

### Example 8

### 3-tert-Butyl-5-(4-chloro-benzoyl)-2,6-dihydroxy-benzoic Acid

3-tert-Butyl-5-(4-chlorobenzoyl)-2,6-dihydroxy-benzoic acid methyl ester (0.194 g) was dissolved in dimethylformamide (4 ml) and this solution was mixed with a solution of sodium thiophenolate (0.350 g) in dimethylformamide (2.65 ml) in a 10 ml microwave vial. This mixture was heated at 100° C. in a microwave reactor for 1 hr and 15 mins and then left at ambient temperature overnight. The reaction mixture was diluted with glacial acetic acid (20 ml) and xylene (50 ml), evaporated to dryness at 70° C. and then diluted with xylene (50 ml) and evaporated to dryness again to give a solid (0.530 g). This solid was suspended in ethyl acetate, filtered and evaporated to give 0.413 g of a solid. A part of this material (0.316 g) was dissolved in dimethylformamide (1 ml) and purified by preparative HPLC on a C8-column using a gradient of ammonium acetate buffer/acetonitrile as eluent. Fractions containing the product were pooled and coevaporated twice from water/actonitrile, dissolved in water and then freeze-dried to give the product as a solid (0.0917 g).

¹H NMR: (400 MHz, METHANOL-d₄) δ ppm 1.37 (s, 9H) 7.41-7.48 (m, 3H) 7.69 (d, 2H); Mass Spectrum: M−H⁺347.

The starting material for this compound was prepared as follows:

### 3-tert-Butyl-5-(4-chloro-benzoyl)-2,6-dihydroxy-benzoic Acid Methyl Ester

Anhydrous aluminium trichloride (0.415 g) was suspended in dry dichloroethane and lumps were crushed using a glass rod. This suspension was stirred in a closed vial at ambient temperature and 4-chlorobenzoyl chloride (0.420 ml) was added and the mixture stirred for 15 minutes to give a clear solution. The reaction mixture was cooled to −10° C. and a solution of 3-tert-Butyl-2,6-dihydroxy-benzoic acid methyl ester (0.350 g) in dry dichloroethane (2 ml) was added and this mixture was stirred at −10-0° C. for 3 days. The reaction mixture was partitioned between dichloromethane (50 ml) and 1M HCl (50 ml) and the organic phase washed twice with 1M HCl (50 ml) and once with water (50 ml). The combined HCl and water extracts were re-extracted with dichlorometane (20 ml) and the combined organic phases were dried and evaporated to give an oil (0.809 g) which was purified by flash chromatography on silica gel using heptane/ethyl acetate (95/5) as eluent. Fractions containing the product were pooled and evaporated to give a solid (0.240 g).

Mass Spectrum: M+H⁺363 and 365.

### 3-tert-Butyl-2,6-dihydroxy-benzoic Acid Methyl Ester

3-tert-Butyl-2,6-dihydroxy-benzoic acid (2.1 g) was dissolved in dry dimethylsulfoxide (4 ml) in a closed vial and triethylamine (3 ml) was added at ambient temperature. Methyl iodide (2.1 ml) was added and the mixture was stirred for 3 days. The reaction mixture was partitioned between ethyl acetate (50 ml) plus toluene (50 ml) and water (50 ml) plus saturated sodium hydrogen carbonate solution (10 ml). The organic phase was washed twice with water (50 ml) plus sodium thiosulphate (0.1 g) and then dried and evaporated to give an oil (2.21 g). This oil was dissolved in toluene (50 ml) plus petroleum ether (20 ml), washed twice with 10% acetic acid, dried and evaporated to give an oil (1.870 g).

¹H NMR: (400 MHz, CHLOROFORM-d) δ ppm 1.38 (s, 9H) 4.09 (s, 3H) 6.43 (d, 1H) 7.37 (d, 1H).

The following compound were synthesised in an analogous method to Example 8.

| Ex | Compound | ¹H NMR | m/z |
|---|---|---|---|
| 9 | 3-tert-butyl-5-(3,4-difluoro- | (400 MHz, METHANOL-d₄) δ ppm | M-H+ |
| | benzoyl)-2,6-dihydroxy-ben- | 1.37 (s, 16 H) 7.27-7.37 (m, 1 H) | 349 |
| | zoic acid | 7.45 (s, 1 H) 7.51-7.57 (m, 1 H) | |
| | | 7.57-7.63 (m, 1 H) | |
| 10 | 3-tert-butyl-2,6-dihydroxy-5- | (400 MHz, METHANOL-d₄) δ ppm | M-H+ |
| | (quinoxaline-2-carbonyl)-ben- | 1.42 (s, 9 H) 7.86-7.94 (m, 2 H) | 365 |
| | zoic acid | 7.95 (s, 1 H) 8.10-8.19 (m, 2 H) | |
| | | 9.06 (s, 1 H) | |
| 11 | 3-(4-chloro-benzoyl)-5-cyclo- | (400 MHz, METHANOL-d₄) δ ppm | M-H+ |
| | hexyl-2,6-dihydroxy-benzoic acid | 1.19-1.52 (m, 5 H) 1.68-1.92 (m, 5 | 373 |
| | | H) 2.80-2.91 (m, 1 H) 7.32 (s, 1 H) | |
| | | 7.41-7.48 (m, 2 H) 7.66-7.72 (m, 2 H) | |

### Example 12

### 3-tert-Butyl-5-[(4-chloro-phenyl)-hydroxyimino-methyl]-2-hydroxy-6-methyl-benzoic Acid

3-tert-Butyl-5-(4-chloro-benzoyl)-2-hydroxy-6-methyl-benzoic acid (0.035 g) was dissolved in dichloroetane (15 ml). Hydroxylamine hydrochloride (0.15 g) and sodium hydrogen carbonate (0.1 g) was added and the mixture was refluxed with a Dean-Stark trap with return of the dense phase for 6 hours. The solvent was slowly distilled off and the mixture heated to dryness at 100° C. overnight. The reaction mixture was dissolved/suspended in methanol (20 ml), filtered and evaporated. The residue (0.07 g) was purified by preparative HPLC on a C8-column using a gradient of ammonium acetate buffer/acetonitrile as eluent. Fractions containing the product were pooled and coevaporated several times from water/actonitrile to give the product as a solid (0.0214 g).

¹H NMR: (400 MHz, METHANOL-d₄) δ ppm 1.33-1.38 (m, 2H) 1.38-1.43 (m, 1H) 1.87-1.98 (m, 9H) 6.80-6.86 (m, 0.7H) 7.09-7.14 (m, 0.3H) 7.27-7.33 (m, 1.4H) 7.33-7.38 (m, 0.6H) 7.40-7.45 (m, 1.4H) 7.56-7.62 (m, 0.6H)

Mass Spectrum: M−H⁺360.

### Example 13

### 5,5′-Di-tert-butyl-4,4′-dihydroxy-2,2′-dimethylbiphenyl-3,3′-dicarboxylic Acid Methylester

5,5′-Di-tert-butyl-4,4′-dihydroxy-2,2′-dimethylbiphenyl-3,3′-dicarboxylic acid dimethylester (51 mg, 0.12 mmol) was dissolved in tetrahydrofuran (0.5 ml), 1 M aqueous potassium hydroxide (0.7 ml) and water (1 ml) and was heated at 70° C. for 1 h. The reaction mixture was acidified with hydrochloric acid, brine was added and the mixture was extracted with dichloromethane (×3). The combined organic phases were dried over magnesium sulfate, filtered and evaporated. The residue was dissolved in dimethylsulfoxide and purified by preparative HPLC to give 5,5′-di-tert-butyl-4,4′-dihydroxy-2,2′-dimethylbiphenyl-3,3′-dicarboxylic acid methylester

¹H NMR (400 MHz, DMSO-d₆) δ ppm 1.33 (s, 9H) 1.34 (s, 9H) 1.91 (s, 3H) 2.10 (s, 3H) 3.85 (s, 3H) 6.88 (s, 1H) 6.95 (s, 1H) 9.84 (s, 1H)

Mass Spectrum: (ESI) 428 (M−H⁺)⁻

The starting material for this compound was prepared as follows:

### 5,5′-Di-tert-butyl-4,4′-dihydroxy-2,2′-dimethylbiphenyl-3,3′-dicarboxylic Acid Dimethylester

3-tert-Butyl-2-hydroxy-6-methylbenzoic acid methyl ester (0.94 g, 4.23 mmol) was dissolved in methanol (20 ml) and sodium bicarbonate (1.07 g, 12.7 mmol) was added. Benzyltrimethylammonium dichloroiodate (1.47 g, 4.23 mmol) was added portion wise over 1 h. The reaction was stirred for one additional hour and then most of the methanol was evaporated. The residue was dissolved in ethyl acetate and was washed with sodium thiosulfate followed by brine. The aqueous phase was extracted twice with ethyl acetate and the combined organic phases were dried over magnesium sulfate, filtered and evaporated. The residue was purified by column chromatography yielding a yellowish solid (0.67 g, 45%).

¹H NMR (400 MHz, CHLOROFORM-d) δ ppm 1.40 (s, 18 H) 2.14 (s, 6H) 3.95 (s, 6H) 7.11 (s, 2H) 11.47 (s, 2H); Mass Spectrum: (EI) 442 (M, 100%), 410 (85%), 395 (28%), 363 (45%), 335 (13%), 189 (13%), 174 (27%)

The following General Procedures were used as indicated in the below Examples 14-32:

General Procedure 1A. Synthesis of Biaryl Ketones.
Methyl 3-bromo-5-tert-butyl-6-methoxy-2-methylbenzoate (52 mg, 0.16 mmol), the boronic acid (0.18 mmol), potassium carbonate (68 mg, 0.50 mmol), and potassium iodide (82 mg, 0.50 mmol) was mixed in anisole (2 mL) and nitrogen was bubbled through the solution for 5 min. After addition of PdCl₂(dppf)₂ (5 mg, 3%) and bubbling with carbon monoxide gas for 5 min, the reaction was heated in a vial under CO atmosphere at 80° C. for 24 hrs. The crude product was evaporated onto silica gel and purified by chromatography (gradient of ethyl acetate in heptane) to give the coupling product.

General Procedure 1B. Synthesis of Biaryl Ketones.
Same procedure as 1A but methyl 3-bromo-5-tert-butyl-6-methoxy-2-methylbenzoate was substituted by methyl 3-tert-butyl-5-iodo-2-methoxy-6-methylbenzoate and potassium iodide was omitted.

General Procedure 2A. Removal of Protecting Groups.
The biaryl ketone was dissolved in dichloromethane and treated with BCl₃ (5-10 eq., 1 M solution in CH₂Cl₂) at −78° C. The temperature was allowed to reach room temperature over 1 to 2 hrs. The excess reagent was decomposed by addition of water and the organic phase separated, dried (MgSO₄) and evaporated. The crude product was heated in the microwave oven with an excess of lithium hydroxide (5-10 eq.) in dimethylformamide/methanol/water (2:1:1) or dimethylformamide/water (3:1, 4 mL) at 150° C. for 10 to 30 min. The crude product was purified by reverse phase chromatography to give the title compounds.

General Procedure 2B. Removal of Protecting Groups.
Procedure as 2A but BCl₃ was decomposed by addition of methanol and the solvents were evaporated in vacuo. The crude product was treated in the microwave oven with two equivalents of lithium hydroxide with respect to the amount BCl₃ used. The crude product was purified by reverse phase chromatography to give the title compounds.

General Procedure 3A. Synthesis of Biaryls.
Methyl 3-bromo-5-tert-butyl-6-methoxy-2-methylbenzoate (50 mg, 0.16 mmol), the boronic acid (0.24 mmol, 1.1 to 1.5 eq.) and potassium fluoride (27 mg, 0.47 mmol) was mixed in toluene (2 mL) and nitrogen was bubbled through the solution for 5 min. After addition of di-tert-butylphosphino pentaphenylferrocene (Q-phos, 11 mg, 10%) and Pd₂(dba)₃ (7 mg, 5%), the reaction was heated in a vial under nitrogen atmosphere at 100° C. for 12 hrs. The crude product was evaporated onto silica gel and purified by chromatography (gradient of EtOAc in heptane) to give the coupling product.

General Procedure 3B. Synthesis of Biaryls.
Methyl 3-bromo-5-tert-butyl-6-methoxy-2-methylbenzoate (50 mg, 0.16 mmol), the boronic acid (0.32 mmol) and potassium phosphate (0.10 g, 0.48 mmol) was mixed in toluene (2 mL) and nitrogen was bubbled through the solution for 5 min. After addition of 2-dicyclohexylphosphino-2′,6′-dimethoxybiphenyl (6 mg, 10%) and Pd₂(dba)₃ (7 mg, 5%), the reaction was heated in a vial under nitrogen atmosphere at 100° C. for 12 hrs. The crude product was evaporated onto silica gel and purified by chromatography (gradient of ethyl acetate in heptane) to give the coupling product.

General Procedure 4A. Synthesis of Diarylamines.
Methyl 3-bromo-5-tert-butyl-6-methoxy-2-methylbenzoate (50 mg, 0.16 mmol), the arylamine (0.16 mmol) and potassium phosphate (51 mg, 0.24 mmol) was mixed in DME (2 mL) and nitrogen was bubbled through the solution for 5 min. After addition of di-tert-butylphosphino pentaphenylferrocene (Q-phos, 11 mg, 10%) and Pd₂(dba)₃ (7 mg, 5%), the reaction was heated in a vial under nitrogen atmosphere at 100° C. for 12 hrs. The crude product was evaporated onto silica gel and purified by chromatography (gradient of ethyl acetate in heptane) to give the product.

### Starting Material:

3-Bromo-5-tert-butyl-6-hydroxy-2-methylbenzoic acid was prepared as set forth in Example 1 of U.S. Pat. No. 4,025,647.

### Example 14

### 3-tert-Butyl-5-(4-fluorobenzoyl)-2-hydroxy-6-methylbenzoic Acid

Procedure 1A was applied using methyl 3-bromo-5-tert-butyl-6-methoxy-2-methylbenzoate (50 mg, 0.16 mmol) and (4-fluorophenyl)boronic acid (25 mg, 0.18 mmol) gave the coupling product (12 mg, 21%). Procedure 2A gave the product (2 mg, 18%).

¹H NMR (400 MHz, METHANOL-d₄) δ ppm 1.38 (s, 9H), 2.41 (s, 3H), 7.19-7.26 (m, 3H), 7.82 (m, 2H). Mass Spectrum: (M−H⁺) 329.

### Example 15

### 3-tert-butyl-2-hydroxy-6-methyl-5-(4-methylbenzoyl)benzoic Acid

Procedure 1A was applied using methyl 3-bromo-5-tert-butyl-6-methoxy-2-methylbenzoate (52 mg, 0.16 mmol) and (4-methylphenyl)boronic acid (24 mg, 0.18 mmol) gave the coupling product (12 mg, 20%). Procedure 2A using tetrahydrofuran/MeOH 3:1 instead of dimethylformamide and starting from methyl 3-tert-butyl-2-methoxy-6-methyl-5-(4-methylbenzoyl)benzoate. (8 mg, 22 μmol) gave the product (3 mg, 42%).

¹H NMR (400 MHz, METHANOL-d₄) δ ppm 1.37 (s, 9H), 2.41 (s, 3H), 2.42 (s, 3H), 7.17 (s, 1H), 7.30 (d, J=8.0 Hz, 2H), 7.65 (d, J=8.0 Hz, 2H).

Mass Spectrum (M−H⁺) 325.

### Example 16

### 3-tert-butyl-5-(3,4-dichlorobenzoyl)-2-hydroxy-6-methylbenzoic Acid

Procedure 1A was applied using methyl 3-bromo-5-tert-butyl-6-methoxy-2-methylbenzoate (0.10 g, 0.32 mmol) and (3,4-dichlorophenyl)boronic acid (69 mg, 0.36 mmol) gave the coupling product (50 mg, 37%). Procedure 2A starting from methyl 3-tert-butyl-5-(3,4-dichlorobenzoyl)-2-methoxy-6-methylbenzoate (22 mg, 54 μmol) gave the product (5 mg, 24%).

¹H NMR (400 MHz, METHANOL-d₄) δ ppm 1.37 (s, 9H), 2.45 (s, 3H), 7.20 (s, 1H), 7.60 (dd, J=8.0, 2.0 Hz, 1H), 7.66 (d, J=8.0 Hz, 1H), 7.88 (d, J=2.0 Hz, 1H).

Mass Spectrum (M−2H⁺) 379.

### Example 17

### 3-tert-butyl-2-hydroxy-6-methyl-5-[4-(trifluoromethyl)benzoyl]benzoic Acid

Procedure 1A was applied using methyl 3-bromo-5-tert-butyl-6-methoxy-2-methylbenzoate (0.10 g, 0.32 mmol) and [4-(trifluoromethyl)phenyl]boronic acid (66 mg, 0.35 mmol) gave the coupling product (27 mg, 21%). Procedure 2A starting from methyl 3-tert-butyl-2-methoxy-6-methyl-5-[4-(trifluoromethyl)benzoyl]benzoate (27 mg, 66 μmol) gave the product (5 mg, 20%).

¹H NMR (400 MHz, METHANOL-d₄) δ ppm 1.37 (s, 9H), 2.45 (s, 3H), 7.24 (s, 1H), 7.81 (d, J=8.0 Hz, 2H), 7.91 (d, J=8.0 Hz, 2H). Mass Spectrum: (M−H⁺) 379.

### Example 18

### 3-tert-butyl-5-(2,4-dichlorobenzoyl)-2-hydroxy-6-methylbenzoic Acid

Procedure 1A was applied using methyl 3-bromo-5-tert-butyl-6-methoxy-2-methylbenzoate (0.10 g, 0.32 mmol) and (2,4-dichlorophenyl)boronic acid (66 mg, 0.35 mmol) gave the coupling product (64 mg, 52%). Procedure 2A starting from methyl 3-tert-butyl-5-(2,4-dichlorobenzoyl)-2-methoxy-6-methylbenzoate (64 mg, 0.15 mmol) gave the product (31 mg, 52%).

¹H NMR (400 MHz, METHANOL-d₄) δ ppm 1.27 (s, 9H), 2.71 (s, 3H), 7.22 (s, 1H), 7.39 (d, J=8 Hz, 1H), 7.44 (dd, J=8, 2 Hz, 1H), 7.57 (d, J=2 Hz, 1H).

Mass Spectrum: (M−2H⁺) 379.

### Example 19

### 3-tert-butyl-2-hydroxy-6-methyl-5-[3-(trifluoromethoxy)benzoyl]benzoic Acid

Procedure 1A was applied using methyl 3-bromo-5-tert-butyl-6-methoxy-2-methylbenzoate (82 mg, 0.26 mmol) and [3-(trifluoromethoxy)phenyl]boronic acid (59 mg, 0.28 mmol) gave the coupling product (43 mg, 39%). Procedure 2A starting from methyl 3-tert-butyl-2-methoxy-6-methyl-5-[3-(trifluoromethoxy)benzoyl]benzoate (43 mg, 0.10 mmol) gave the product (22 mg, 55%).

¹H NMR (400 MHz, METHANOL-d₄) δ ppm 1.36 (s, 9H), 2.48 (s, 3H), 7.17 (s, 1H), 7.52 (m, 1H), 7.57-7.64 (m, 2H), 7.71 (m, 1H). Mass spectrum: (M−H⁺) 395.

### Example 20

### 3-tert-butyl-2-hydroxy-5-(3-isopropylbenzoyl)-6-methylbenzoic Acid

Procedure 1A was applied using methyl 3-bromo-5-tert-butyl-6-methoxy-2-methylbenzoate (0.11 g, 0.34 mmol) and (3-isopropylphenyl)boronic acid (61 mg, 0.37 mmol) gave the coupling product (47 mg, 36%). Procedure 2A starting from methyl 3-tert-butyl-5-(3-isopropylbenzoyl)-2-methoxy-6-methylbenzoate (47 mg, 0.12 mmol) gave the product (9 mg, 21%).

¹H NMR (400 MHz, METHANOL-d₄) δ ppm 1.26 (d, J=7 Hz, 6H), 1.36 (s, 9H), 2.47 (s, 3H), 2.96 (heptet, J=7 Hz, 1H), 7.15 (s, 1H), 7.40 (m, 1H), 7.49 (m, 1H), 7.56 (m, 1H), 7.62 (m, 1H). Mass spectrum: (M−H⁺) 353.

### Example 21

### 3-tert-butyl-2-hydroxy-6-methyl-5-(3-nitrobenzoyl)benzoic Acid

Procedure 1B was applied using methyl 3-tert-butyl-5-iodo-2-methoxy-6-methylbenzoate (0.22 g, 0.62 mmol) and (3-nitrophenyl)boronic acid (0.11 g, 0.68 mmol) gave the coupling product (0.12 g, 52%). Procedure 2B starting from methyl 3-tert-butyl-2-methoxy-6-methyl-5-(3-nitrobenzoyl)benzoate (62 mg, 0.16 mmol) gave the product (19 mg, 33%).

¹H NMR (400 MHz, METHANOL-d₄) δ ppm 1.36 (s, 9H), 2.49 (s, 3H), 7.21 (s, 1H), 7.75 (m, 1H), 8.08 (m, 1H), 8.45 (m, 1H), 8.56 (m, 1H). Mass spectrum: (M−H⁺) 356.

### Example 22

### 3-tert-butyl-2-hydroxy-5-(2-hydroxybenzoyl)-6-methylbenzoic Acid

Procedure 1B was applied using methyl 3-tert-butyl-5-iodo-2-methoxy-6-methylbenzoate (0.10 g, 0.27 mmol) and (2-methoxyphenyl)boronic acid (46 mg, 0.30 mmol) gave the coupling product (47 mg, 47%). Procedure 2B starting from methyl 3-tert-butyl-2-methoxy-5-(2-methoxybenzoyl)-6-methylbenzoate (47 mg, 0.13 mmol) gave the product (24 mg, 57%).

¹H NMR (400 MHz, METHANOL-d₄) δ ppm 1.38 (s, 9H), 2.43 (s, 3H), 6.84 (m, 1H), 6.99 (d, J=8.5 Hz, 1H), 7.09 (s, 1H), 7.33 (dd, J=8.0, 1.5 Hz, 1H), 7.49 (m, 1H). Mass spectrum: (M−H⁺) 327.

### Example 23

### 3-tert-butyl-2-hydroxy-6-methyl-5-[2-(trifluoromethyl)benzoyl]benzoic Acid

Procedure 1B was applied using methyl 3-tert-butyl-5-iodo-2-methoxy-6-methylbenzoate (0.10 g, 0.27 mmol) and [2-(trifluoromethyl)phenyl]boronic acid (57 mg, 0.30 mmol) gave the coupling product (27 mg, 24%). Procedure 2B starting from methyl 3-tert-butyl-2-methoxy-6-methyl-5-[2-(trifluoromethyl)benzoyl]benzoate (27 mg, 66 μmol) gave the product (11 mg, 43%).

¹H NMR (400 MHz, METHANOL-d₄) δ ppm 1.22 (s, 9H), 2.75 (s, 3H), 7.17 (s, 1H), 7.40 (m, 1H), 7.63-7.72 (m, 2H), 7.81 (m, 1H). Mass spectrum: (M−H⁺) 379.

### Example 24

### 5-tert-butyl-4-hydroxy-2-methylbiphenyl-3-carboxylic Acid

Procedure 3A was applied using methyl 3-bromo-5-tert-butyl-6-methoxy-2-methylbenzoate (0.10 g, 0.32 mmol) and phenylboronic acid (43 mg, 0.35 mmol) gave the coupling product (82 mg, 82%). Procedure 2A gave the product (5 mg, 7%).

¹H NMR (400 MHz, METHANOL-d₄) δ ppm 1.39 (s, 9H), 2.35 (s, 3H), 7.07 (s, 1H), 7.20-7.30 (m, 4H), 7.33-7.39 (m, 2H). Mass spectrum: (M−H⁺) 283.

### Example 25

### 5-tert-butyl-4-hydroxy-2,2′-dimethylbiphenyl-3-carboxylic Acid

Procedure 3A was applied using methyl 3-bromo-5-tert-butyl-6-methoxy-2-methylbenzoate (50 mg, 0.16 mmol) and (2-methylphenyl)boronic acid (33 mg, 0.24 mmol) gave the coupling product (13 mg, 25%). Procedure 2A gave the product (4 mg, 33%).

¹H NMR (400 MHz, METHANOL-d₄) δ ppm 1.38 (s, 9H), 2.02 (s, 3H), 2.18 (s, 3H), 6.87 (s, 1H), 7.02 (m, 1H), 7.13-7.24 (m, 3H). Mass spectrum: (M−H⁺) 297.

### Example 26

### 5-tert-butyl-4-hydroxy-4′-methoxy-2,2′-dimethylbiphenyl-3-carboxylic Acid

Procedure 3B was applied using methyl 3-bromo-5-tert-butyl-6-methoxy-2-methylbenzoate (50 mg, 0.16 mmol) and (4-methoxy-2-methylphenyl)boronic acid (53 mg, 0.32 mmol) gave the coupling product (63 mg, quant.). Procedure 2B starting from methyl 5-tert-butyl-4,4′-dimethoxy-2,2′-dimethylbiphenyl-3-carboxylate (0.10 g, 0.30 mmol) gave the product (37 mg, 39%).

¹H NMR (400 MHz, METHANOL-d₄) δ ppm 1.38 (s, 9H), 1.99 (s, 3H), 2.19 (s, 3H), 3.80 (s, 3H), 6.74 (dd, J=8, 2.5 Hz, 1H), 6.80 (d, J=2.5 Hz, 1H), 6.91 (s, 1H), 6.93 (d, J=8 Hz, 1H). Mass spectrum: (M−H⁺) 327.

### Example 27

### 5-tert-butyl-4,4′-dihydroxy-2,2′-dimethylbiphenyl-3-carboxylic Acid

Procedure as in Example 26 (i.e. for 5-tert-butyl-4-hydroxy-4′-methoxy-2,2′-dimethylbiphenyl-3-carboxylic acid) but methyl 5-tert-butyl-4,4′-dimethoxy-2,2′-dimethylbiphenyl-3-carboxylate (85 mg, 0.24 mmol) was treated with BCl₃ at 25° C. for 3 days, which subsequently gave the product (40 mg, 53%).

¹H NMR (400 MHz, METHANOL-d₄) δ ppm 1.39 (s, 9H), 1.95 (s, 3H), 2.18 (s, 3H), 6.61 (dd, J=8, 2.5 Hz, 1H), 6.68 (d, J=2.5 Hz, 1H), 6.83 (d, J=8 Hz, 1H), 6.87 (s, 1H). Mass spectrum: (M−H⁺) 313.

### Example 28

### 5-tert-butyl-4-hydroxy-4′-methoxy-2-methylbiphenyl-3-carboxylic Acid

Procedure 3B was applied using methyl 3-bromo-5-tert-butyl-6-methoxy-2-methylbenzoate (50 mg, 0.16 mmol) and (4-methoxyphenyl)boronic acid (49 mg, 0.32 mmol) gave the coupling product (48 mg, 88%.). Procedure 2B with BCl₃ treatment at 25° C. for 3 days gave the product (13 mg, 29%).

¹H NMR (400 MHz, METHANOL-d₄) δ ppm 1.39 (s, 9H), 2.35 (s, 3H), 3.82 (s, 3H), 6.92 (m, 2H), 7.04 (s, 1H), 7.14 (m, 2H). Mass spectrum: (M−H⁺) 313.

### Example 29

### 5-tert-butyl-4-hydroxy-3′-isopropyl-2-methylbiphenyl-3-carboxylic Acid

Procedure 3B was applied using methyl 3-bromo-5-tert-butyl-6-methoxy-2-methylbenzoate (50 mg, 0.16 mmol) and (3-isopropylphenyl)boronic acid (52 mg, 0.32 mmol) gave the coupling product (64 mg, quant.). Procedure 2A gave the product (29 mg, 49%).

¹H NMR (400 MHz, METHANOL-d₄) δ ppm 1.27 (d, J=7 Hz, 6H), 1.40 (s, 9H), 2.35 (s, 3H), 2.92 (heptet, J=7 Hz, 1H), 7.02 (m, 1H), 7.08 (s, 1H), 7.16 (m, 1H), 7.28 (m, 1H). Mass spectrum: (M−H⁺) 325.

### Example 30

### 3′,5-di-tert-butyl-4-hydroxy-2,5′-dimethylbiphenyl-3-carboxylic Acid

Procedure 3B was applied using methyl 3-bromo-5-tert-butyl-6-methoxy-2-methylbenzoate (50 mg, 0.16 mmol) and (3-tert-butyl-5-methylphenyl)boronic acid (61 mg, 0.32 mmol) gave the coupling product (64 mg, quant.). Procedure 2A gave the product (40 mg, 70%).

¹H NMR (400 MHz, METHANOL-d₄) δ ppm 1.32 (s, 9H), 1.40 (s, 9H), 2.34 (s, 3H), 2.36 (s, 3H), 6.85 (m, 1H), 7.02 (s, 1H), 7.04 (m, 1H), 7.14 (m, 1H).

Mass spectrum: (M−H⁺) 353.

### Example 31

### 3-anilino-5-tert-butyl-6-hydroxy-2-methylbenzoic acid

Procedure 4A was applied using methyl 3-bromo-5-tert-butyl-6-methoxy-2-methylbenzoate (50 mg, 0.16 mmol) and aniline (14 μL, 0.16 mmol) gave the coupling product (16 mg, 30%). Procedure 2B gave the product (3 mg, 20%).

¹H NMR (400 MHz, METHANOL-d₄) δ ppm 1.37 (s, 9H), 2.37 (s, 3H), 6.52-6.62 (m, 3H), 7.02-7.08 (m, 3H). Mass spectrum: (M−H⁺) 298.

### Example 32

### 3-tert-butyl-5-[(4-chlorophenyl)amino]-2-hydroxy-6-methylbenzoic Acid

Procedure 4A was applied using methyl 3-bromo-5-tert-butyl-6-methoxy-2-methylbenzoate (0.10 g, 0.32 mmol) and 4-chloroaniline (41 mg, 0.32 mmol) gave the coupling product (68 mg, 59%). Procedure 2B from methyl 3-tert-butyl-5-[(4-chlorophenyl)amino]-2-methoxy-6-methylbenzoate (40 mg, 0.11 mmol) gave the product (11 mg, 30%).

¹H NMR (400 MHz, METHANOL-d₄) δ ppm 1.37 (s, 9H), 2.36 (s, 3H), 6.49 (m, 2H), 7.02 (m, 2H), 7.08 (s, 1H). Mass spectrum: (M−H⁺) 332.

### Example 33

### 3-tert-butyl-5-[(4-chlorophenyl)(methyl)amino]-2-hydroxy-6-methylbenzoic Acid

The intermediate product methyl 3-tert-butyl-5-[(4-chlorophenyl)amino]-2-methoxy-6-methylbenzoate (28 mg, 77 μmol) from example 32 was dissolved in MeCN (1 mL) with sodium cyanoborohydride (15 mg, 0.23 mmol) and formaldehyde (58 μL, 37% (aq.), 0.77 mmol). Acetic acid (16 μL) was added in two portions, two hours apart. The reaction was worked up by extraction (CH₂Cl₂/K₂CO₃ (aq.)) after 12 h to give the methylated product (30 mg, quant.). Procedure 2B gave the product (9 mg, 33%).

¹H NMR (400 MHz, METHANOL-d₄) δ ppm 1.37 (s, 9H), 2.28 (s, 3H), 3.14 (s, 3H), 6.40 (m, 2H), 7.06 (m, 3H). Mass spectrum: (M−H⁺) 346.

### Example 34

### 3-tert-Butyl-5-[5-(4-chlorophenyl)-[1,2,4]oxadiazol-3-yl]-2-hydroxy-6-methylbenzoic Acid

3-tert-Butyl-5-[5-(4-chlorophenyl)-[1,2,4]oxadiazol-3-yl]-2-methoxy-6-methylbenzoic acid methyl ester (15 mg, 0.036 mmol) was dissolved in anhydrous dichloromethane. The mixture was put under nitrogen atmosphere and cooled at −72° C. Borontrichloride (1M in dichloromethane, 0.36 ml, 0.36 mmol) was added drop wise. The reaction was kept at −72° C. for 1 h. The cooling bath was removed and the reaction was quenched by the addition of water. The phases were separated and the aqueous phase was extracted with dichloromethane (×2). The organic phases were combined and the solvent was removed in vacuo. The residue was dissolved in a mixture of dimethylformamide (1 ml), methanol (0.5 ml) and water (0.2 ml). Lithium hydroxide monohydrate (15 mg, 0.36 mmol) was added and the reaction mixture was heated at 70° C. for 3 h. The mixture was concentrated in vacuo and purified by reverse phase liquid chromatography to give a pink solid (1.1 mg, 8%)

¹H NMR (400 MHz, METHANOL-d₄) δ ppm 1.43 (s, 9H) 2.73 (s, 3H) 7.63 (d, J=8.84 Hz, 2H) 7.74 (s, 1H) 8.19 (d, J=8.59 Hz, 2H) Mass spectrum: (ESI) 385 (M−H⁺)⁻, 387 (M−H⁺)⁻

The starting materials for this compound were synthesized as follows:

### 3-tert-Butyl-5-cyano-2-methoxy-6-methylbenzoic Acid Methyl Ester

3-Bromo-5-tert-butyl-6-methoxy-2-methylbenzoic acid methyl ester (205 mg, 0.65 mmol) was dissolved in anhydrous dimethylformamide (1.5 ml) and copper cyanide (76 mg, 0.85 mmol) was added. The reaction was heated at 160° C. for 3 h. The reaction mixture was purified by column chromatography on silica eluting with a gradient of 10-25% ethylacetate in heptane yielding 120 mg (71%) of a brown oil.

¹H NMR (400 MHz, CHLOROFORM-d) δ ppm 1.34 (s, 9H) 2.42 (s, 3H) 3.82 (s, 3H) 3.95 (s, 3H) 7.57 (s, 1H). ¹³C NMR (101 MHz, CHLOROFORM-d) δ ppm 17.7, 30.2, 35.1, 52.8, 61.5, 107.8, 118.1, 128.7, 132.5, 139.3, 141.8, 160.4, 167.9.

Mass spectrum: (EI) 261 (M, 21%), 214 (100%).

### 3-tert-Butyl-5-(N-hydroxycarbamimidoyl)-2-methoxy-6-methylbenzoic Acid Methyl Ester

3-tert-Butyl-5-cyano-2-methoxy-6-methylbenzoic acid methyl ester (120 mg, 0.46 mmol) was dissolved in absolute ethanol (5 ml) and hydroxylamine hydrochloride (108 mg, 1.56 mmol) followed by sodium bicarbonate (132 mg, 1.56 mmol) was added. The reaction mixture was heated at reflux for 20 h. The solvent was evaporated and the residue was partitioned between aqueous sodium bicarbonate and dichloromethane. The aqueous phase was extracted with dichloromethane (×2). The combined organic phase was dried (MgSO₄), concentrated and purified by column chromatography on silica eluting with ethyl acetate:heptane 1:1 to give a white solid (160 mg) that was used without further purification (66% pure according to LC-UV 254 nm).

¹H NMR (400 MHz, CHLOROFORM-d) δ ppm 1.36 (s, 9H) 2.33 (s, 3H) 3.79 (s, 3H) 3.94 (s, 3H) 7.43 (s, 1H) Mass spectrum: (ESI) 295 (M+H)⁺

### Methyl 3-(amino{[(4-chlorobenzoyl)oxy]imino}methyl)-5-tert-butyl-6-methoxy-2-methylbenzoate

3-tert-Butyl-5-(N-hydroxycarbamimidoyl)-2-methoxy-6-methylbenzoic acid methyl ester (50 mg, 0.17 mmol) was dissolved in anhydrous dimethylformamide (1 ml) and diisopropylethylamine (23 μl, 0.25 mmol) was added. The mixture was cooled on ice-water, put under nitrogen atmosphere, and 4-chlorobenzoylchloride (22 μL, 0.17 mmol) was added dropwise. The reaction mixture was stirred at 0° C. for 30 min and at room temperature for 30 min. The reaction mixture was concentrated by evaporation and purified by column chromatography eluting with 25% ethyl acetate in heptane to give 23 mg of the title compound as a dry film (37% from 3-tert-butyl-5-cyano-2-methoxy-6-methylbenzoic acid methyl ester).

¹H NMR (400 MHz, CHLOROFORM-d) δ ppm 1.36 (s, 9H) 2.34 (s, 3H) 3.79 (s, 3H) 3.94 (s, 3H) 7.42 (s, 1H) 7.45 (d, J=8.59 Hz, 2H) 8.02 (d, J=8.59 Hz, 2H)

Mass spectrum: (ESI) 433; 435 (M+H)⁺

### 3-tert-Butyl-5-[5-(4-chlorophenyl)-[1,2,4]oxadiazol-3-yl]-2-methoxy-6-methylbenzoic Acid Methyl Ester

Methyl 3-(amino {[(4-chlorobenzoyl)oxy]imino}methyl)-5-tert-butyl-6-methoxy-2-methylbenzoate (23 mg, 0.053 mmol) was dissolved in anhydrous dimethylformamide (5 ml) and was heated at 120° C. for 3 h until all starting material was consumed. The solvent was evaporated and the product was isolated as a dry film (15 mg, 68%) using column chromatography on silica eluting with 10% ethyl acetate in heptane.

¹H NMR (400 MHz, CHLOROFORM-d) δ ppm 1.42 (s, 9H) 2.50 (s, 3H) 3.85 (s, 3H) 3.97 (s, 3H) 7.53 (d, J=8.84 Hz, 2H) 7.99 (s, 1H) 8.14 (d, J=8.59 Hz, 2H)

¹³C NMR (101 MHz, CHLOROFORM-d) δ ppm 17.9, 30.6, 35.0, 52.6, 62.0, 121.6, 122.7, 129.5, 129.5, 130.5, 134.9, 140.8 Mass spectrum: (ESI) 415 (M+H⁺)⁺, 417 (M+H⁺)⁺

### Example 35

### 3-tert-butyl-2-hydroxy-5-[(4-methoxyphenyl)thio]-6-methylbenzoic Acid

A solution of 3-tert-butyl-2-hydroxy-6-methyl-benzoic acid (120 mg, 0.58 mmol) and anhydrous pyridine (117 μL, 1.4 mmol) in anhydrous dichloromethane (3 ml) was cooled to 0° C. under N₂-atmosphere. The solution of 4-methoxybenzenesulfenyl chloride was added dropwise via syringe and then stirred at 0° C. for 1.5 hours. The reaction mixture was stored at −18° C. for two days. The organic phase was washed with water (2×5 ml), brine (1×5 ml), dried (MgSO₄), filtered and the solvent was evaporated. The product was purified by preparative HPLC on a C8-column using a gradient of ammonium acetate buffer/acetonitrile as eluent. Fractions containing the product were pooled and coevaporated from water/actonitrile, dissolved in water and then freeze-dried to give the title compound as a solid (55 mg, 28% yield).

¹H NMR (400 MHz, CHLOROFORM-d) δ ppm 1.35-1.43 (m, 9H) 2.43 (s, 3H) 3.86 (s, 3H) 6.93-6.96 (m, 2H) 7.73-7.76 (m, 2H); Mass Spectrum (ESI): M−H 345.

The starting material for this compound was synthesized as follows:

### 4-methoxybenzenesulfenyl Chloride

To a rapidly stirred suspension of N-chlorosuccinimide (97 mg, 0.7 mmol) in anhydrous dichloromethane (3 ml) was 4-methoxy benzenethiol (97 mg, 0.7 mmol) in anhydrous dichloromethane (3 ml) added dropwise via syringe under N₂-atmosphere at room temperature. Initiation of product formation was indicated by the intense red coloration of the reaction mixture. The mixture was stirred for 30 min after final addition of the thiol. The final solution was used crude in the upcoming step.

The following compounds were synthesised in analogy with the above procedure for synthezising 4-methoxybenzenesulfenyl chloride, and used in the following examples as indicated below: Naphthalene-1-sulfenyl chloride (used in example 36, 39, and 41) was synthesized in analogy with 4-methoxybenzenesulfenyl chloride starting from naphthalene-1-thiol and with the exception that thionyl chloride was used as chlorinating agent instead of N-chlorosuccinimide.

4-Phenyl-1,3-thiazole-2-sulfenyl chloride (used in example 40) was synthesized in analogy with 4-methoxybenzenesulfenyl chloride starting from 2-phenyl-1,3-thiazole-4-thiol and with the exception that thionyl chloride was used as chlorinating agent instead of N-chlorosuccinimide.

2,4-Dichlorobenzenesulfenyl chloride (Used in example 37, 38 and 42) was synthesized in analogy with 4-methoxybenzenesulfenyl chloride starting from 2,4-dichlorobenzenethiol. After 30 min stirring the reaction mixture was evaporated at 60° C. and 300 mbar for 30 min to obtain the product as a liquid.

### Example 36

### 3-tert-butyl-2-hydroxy-6-methyl-5-(1-naphthylthio)benzoic Acid

The title compound was prepared and isolated as a solid (94 mg, 53% yield) in analogy with example 35, using naphthalene-1-sulfenyl chloride instead of 4-methoxybenzenesulfenyl chloride.

¹H NMR (400 MHz, CHLOROFORM-d) δ ppm 1.34 (s, 9H) 2.72 (s, 3H) 6.92 (d, 1H) 7.28-7.33 (m, 1H) 7.53-7.58 (m, 2H) 7.59 (s, 1H) 7.67 (d, 1H) 7.85-7.89 (m, 1H) 8.32 (d, 1H); Mass Spectrum (ESI): M−H⁺365.

### Example 37

### 3-[(2,4-dichlorophenyl)thio]-6-hydroxy-5-isopropyl-2-methylbenzoic Acid

The title compound was prepared and isolated as a gum (117 mg, 41% yield) in analogy with example 35 (dichloroethane was used as solvent), using 2,4-dichlorobenzenesulfenyl chloride instead of 4-methoxybenzenesulfenyl chloride.

¹H NMR (400 MHz, DMSO-d₆) δ ppm 1.11 (d, 6H) 2.58 (s, 3H) 3.12-3.25 (m, 1H) 6.38 (d, 1H) 7.17 (s, 1H) 7.26 (dd, 2H) 7.58 (d, 1H); Mass Spectrum (ESI): M−H⁺369.

### Example 38

### 3-tert-butyl-5-[(2,4-dichlorophenyl)thio]-2,6-dihydroxybenzoic Acid

The title compound was prepared and isolated as a gum (273 mg, 69% yield) in analogy with example 35 (dichloroethane was used as solvent), using 2,4-dichlorobenzenesulfenyl chloride instead of 4-methoxybenzenesulfenyl chloride.

¹H NMR (400 MHz, DMSO-d₆) δ ppm 1.31 (s, 9H) 6.54 (d, 1H) 7.14 (s, 1H) 7.26 (dd, 1H) 7.54 (d, 1H) 15.68 (s, 1H) 16.07 (s, 1H); Mass Spectrum (ESI): M−H⁺385.

### Example 39

### 2-hydroxy-3-isopropyl-6-methyl-5-(1-naphthylthio)benzoic Acid

The title compound was prepared and isolated as an oil (16 mg, 6% yield) in analogy with example 35 (dichloroethane was used as solvent), using naphthalene-1-sulfenyl chloride instead of 4-methoxybenzenesulfenyl chloride.

¹H NMR (400 MHz, DMSO-d₆) δ ppm 1.10 (d, 6H) 2.56-2.62 (m, 3H) 3.15-3.25 (m, 1H) 6.67-6.74 (m, 1H) 7.18-7.24 (m, 1H) 7.29-7.38 (m, 1H, overlapping with signal from ammonium acetate) 7.54-7.64 (m, 2H) 7.64-7.71 (m, 1H) 7.94 (d, 1H) 8.21 (d, 1H); Mass Spectrum (ESI): M−H⁺351.

### Example 40

### 3-tert-butyl-2-hydroxy-6-methyl-5-[(4-phenyl-1,3-thiazol-2-yl)thio]benzoic Acid

The title compound was prepared and isolated as a solid (26 mg, 14% yield) in analogy with example 35, using 4-Phenyl-1,3-thiazole-2-sulfenyl chloride instead of 4-methoxybenzenesulfenyl chloride.

¹H NMR (400 MHz, CHLOROFORM-d) δ ppm 1.42 (s, 9H) 2.82 (s, 3H) 7.26 (s, 1H) 7.30-7.35 (m, 1H) 7.38-7.44 (m, 2H) 7.80-7.88 (m, 3H); Mass Spectrum (ESI): M−H⁺398.

### Example 41

### 3-tert-butyl-2,6-dihydroxy-5-(1-naphthylthio)benzoic Acid

The title compound was prepared and isolated as a solid (64 mg, 18% yield) in analogy with example 35 (dichloroethane was used as solvent), using naphthalene-1-sulfenyl chloride instead of 4-methoxybenzenesulfenyl chloride.

¹H NMR (400 MHz, DMSO-d₆) δ ppm 1.29 (s, 9H) 6.88 (d, 1H) 7.15 (s, 1H) 7.33 (m, 1H) 7.51-7.61 (m, 2H) 7.65 (d, 1H) 7.89-7.94 (m, 1H) 8.23 (d, 1H) 15.57 (s, 1H) 16.01 (s, 1H); Mass Spectrum (ESI): M−H⁺367.

### Example 42

### 3-tert-butyl-5-[(2,4-dichlorophenyl)thio]-2-hydroxy-6-methylbenzoic Acid

The title compound was prepared and isolated as a solid (76 mg, 19% yield) in analogy with example 35, using 2,4-dichlorobenzenesulfenyl chloride instead of 4-methoxybenzenesulfenyl chloride.

¹H NMR (400 MHz, METHANOL-d₄) δ ppm 1.39 (s, 9H) 2.60 (s, 3H) 6.46 (d, 1H) 7.13 (dd, 1H) 7.43 (d, 1H) 7.58 (s, 1H); Mass spectrum (ESI): 383 M−H⁺.

### Example 43

### 3-Benzylsulfanyl-5-tert-butyl-6-hydroxy-2-methylbenzoic acid

Benzyl mercaptan (42 μl, 0.36 mmol) was dissolved in i-propanol (1 ml). Sodium borohydride (9 mg, 0.24 mmol) was added and the mixture was stirred under argon atmosphere at room temperature for 1.5 h. 3-tert-Butyl-2-hydroxy-5-iodo-6-methylbenzoic acid (100 mg, 0.30 mmol), copper iodide (11 mg, 0.06 mmol), ethylene glycol (37 mg, 0.60 mmol) and potassium carbonate (83 mg, 0.60 mmol) were mixed in i-propanol and the solution of benzyl mercaptan was added. The reaction mixture was heated at 80° C. under argon atmosphere for 16 h. Benzyl mercaptan (25 μl, 0.21 mmol), copper iodide (35 mg, 0.18 mmol), ethylene glycol (37 mg, 0.6 mmol) and potassium carbonate (40 mg, 0.29 mmol) was added and the mixture was refluxed for 3 h under argon atmosphere. The reaction mixture was filtered into and the filtrate was partitioned between dichloromethane and aqueous sodium bicarbonate. The aqueous phase was extracted twice with dichloromethane and the combined organic phases were dried over magnesium sulfate and concentrated. The residue was dissolved in dimethylsulfoxide and purified by prep-HPLC to give 12 mg (12%) of a dry film.

¹H NMR (400 MHz, CHLOROFORM-d) δ ppm 1.23 (s, 9H) 2.59 (s, 3H) 3.79 (s, 2H) 7.02-7.10 (m, 2H) 7.11-7.21 (m, 4H); Mass spectrum: (ESI) 329 (M−H⁺)⁻

### Example 43B

### 3-Benzylsulfanyl-5-tert-butyl-6-hydroxy-2-methylbenzoic Acid, Alternative Procedure for Preparation

Argon was bubbled through anhydrous dimethylformamide (4 ml) for 15 min and the solution was transferred to a round bottled charged with 3-tert-butyl-2-hydroxy-5-mercapto-6-methyl-benzoic acid (96 mg, 0.4 mmol) and NaHCO₃ (101 mg, 1.2 mmol) under argon atmosphere. To the resulting mixture was benzyl bromide (273 mg, 1.6 mmol) added via syringe and the solution was stirred at ambient temperature for 1 h. The solvent was evaporated and water (10 ml) was added. The water phase was extracted with ethyl acetate (3×5 ml) and the combined organic phases were washed with brine (15 ml), dried (MgSO₄), filtered and the solvent was evaporated to give an oil (466 mg) which was purified by preparative HPLC on a C8-column using a gradient of ammonium acetate buffer/acetonitrile as eluent. Fractions containing the product were pooled and coevaporated from water/actonitrile, dissolved in water and then freeze-dried to give the product as a gum (24 mg, 18% yield).

¹H NMR: (400 MHz, DMSO-d₆) δ ppm 1.20 (s, 9H) 2.69 (s, 3H) 3.77 (s, 2H) 6.84 (s, 1H) 7.06-7.10 (m, 2H) 7.16-7.24 (m, 2H); Mass Spectrum (ESI): M−H⁺329.

The starting material for this compound was prepared as follows:

### 3-tert-butyl-2-hydroxy-6-methyl-5-thiocyanato-benzoic Acid

To a solution of 3-tert-butyl-2-hydroxy-6-methyl-benzoic acid (1.0 g, 4.8 mmol) and sodium thiocyanate (1.2 g, 14.4 mmol) in anhydrous methanol (14 ml) was bromine (0.77 g, 4.80 mmol) dissolved in anhydrous methanol (14 ml) added dropwise at 0° C. The solvent was evaporated immediately after complete addition of bromine. The crude product was dissolved in dichloromethane (20 ml) and the organic phase was washed with water (2×20 ml), brine (20 mL), dried (MgSO₄), filtered and the solvent was evaporated. The product was purified by preparative HPLC on a C8-column using a gradient of ammonium acetate buffer/acetonitrile as eluent. Fractions containing the product were pooled and coevaporated from water/actonitrile, dissolved in water and then freeze-dried to give the product as a solid (0.65 g, 51% yield).

¹H NMR: (400 MHz, CHLOROFORM-d) δ ppm 1.31 (s, 9H) 2.75 (s, 3H) 7.42 (s, 1H);

Mass Spectrum (ESI): M−H⁺264

### 3-tert-butyl-2-hydroxy-5-mercapto-6-methyl-benzoic Acid

Argon was bubbled through the mixture of ethanol (3 ml) and aqueous KH₂PO₄ (3 ml, 0.2 M) solution for 15 min. To this solution were 3-tert-butyl-2-hydroxy-6-methyl-5-thiocyanato-benzoic acid (0.41 g, 1.6 mmol) and DL-dithiothreitol (0.36 g, 2.3 mmol) added and the resulting mixture was stirred at 50° C. for 2 hours and then left at ambient temperature overnight. The ethanol was evaporated and the aqueous phase was extracted with ethyl acetate (2×15 ml). The aqueous phase was acidified to pH 3 with 2M HCl and extracted with ethyl acetate. The combined organic phases were washed with brine (20 ml), dried (MgSO₄), filtered and the solvent was evaporated to give 0.81 g of the crude product as a white solid. The product was used crude in upcoming steps.

Mass Spectrum (ESI): M−H⁺239.

### Example 44

### 3-tert-butyl-5-[(2,3-difluorobenzyl)thio]-2-hydroxy-6-methylbenzoic Acid

The title compound was prepared and isolated as a solid (55 mg, 30% yield) in analogy with Example 43B (the reaction was performed in a Radley carousel) using 2,3-difluorobenzyl bromide as the alkylating agent instead of benzylbromide.

¹H NMR (400 MHz, DMSO-d₆) δ ppm 1.18 (s, 9H) 2.65 (s, 3H) 3.85 (s, 2H) 6.78-6.86 (m, 2H) 6.99-7.06 (m, 1H) 7.19-7.29 (m, 1H); Mass Spectrum (ESI): M−H⁺365.

### Example 45

### 3-tert-butyl-5-[(4-chlorobenzyl)thio]-2-hydroxy-6-methylbenzoic Acid

The title compound was prepared and isolated as a solid (39 mg, 21% yield) in analogy with Example 43B (the reaction was performed in a Radley carousel) using 4-chlorobenzyl bromide as the alkylating agent instead of benzylbromide.

¹H NMR (400 MHz, DMSO-d₆) δ ppm 1.19 (s, 9H) 2.69 (s, 3H) 3.75 (s, 2H) 6.75 (s, 1H) 7.01-7.06 (m, 2H) 7.21-7.26 (m, 2H); Mass Spectrum (ESI): M−H⁺363.

### Example 46

### 3-tert-Butyl-2-hydroxy-6-methyl-5-phenylmethanesulfinylbenzoic Acid

3-tert-Butyl-2-hydroxy-5-iodo-6-methylbenzoic acid (90 mg, 0.27 mmol), copper iodide (10 mg, 0.05 mmol), ethylene glycol (34 mg, 0.54 mmol), benzyl mercaptan (35 μl, 0.30 mmol) and potassium carbonate (112 mg, 0.81 mmol) were mixed in i-propanol. The reaction mixture was heated at 90° C. under argon atmosphere for 5 h. The mixture was filtered, diluted with aqueous sodium bicarbonate and extracted (×3) with dichloromethane. The organic phases were concentrated to dryness. 50% of the crude was dissolved in acetic acid (0.5 ml) and 30% hydrogen peroxide-solution in water (25 μl) was added. The reaction mixture was heated at 90° C. for 1 hour. the solvent was removed by evaporation and the residue was dissolved in dimethylsulfoxide and purified by preparative HPLC. The title compound (3.6 mg, 8%) was obtained as a white solid.

¹H NMR (400 MHz, METHANOL-d₄) δ ppm 1.21 (s, 9H) 2.43 (s, 3H) 3.95 (d, J=13.0 Hz, 1H) 4.08 (d, J=13.0 Hz, 1H) 6.86 (d, J=6.8 Hz, 2H) 7.11-7.25 (m, 4H)

Mass spectrum: (ESI) 347 (M+H⁺)⁺

### Example 47

### 3-tert-Butyl-2-hydroxy-6-methyl-5-phenylmethanesulfonylbenzoic Acid

3-Benzylsulfanyl-5-tert-butyl-6-hydroxy-2-methylbenzoic acid (12 mg, 36 μmol) was dissolved in acetic acid (1 ml). 30% Hydrogen peroxide-solution in water (35 μl) was added and the mixture was heated at 90° C. for 30 min until all starting material was consumed according to LC-MS. The mixture was concentrated by evaporation and the residue was diluted with methanol and purified by prep-LC to give 3.5 mg (27%) of a dry film.

¹H NMR (400 MHz, METHANOL-d₄) δ ppm 1.14 (s, 9H) 2.85 (s, 3H) 4.34 (s, 2H) 7.00 (m, 2H) 7.11-7.24 (m, 4H);

¹³C NMR (101 MHz, METHANOL-d₄) δ ppm 18.8, 29.5, 35.5, 62.9, 125.8, 129.3, 129.3, 130.7, 131.7, 132.1, 135.0, 140.7, 165.4; Mass spectrum: (ESI) 361 (M−H⁺)⁻

### Example 48

### 3-tert-butyl-2-hydroxy-5-[(4-methoxyphenyl)sulfonyl]-6-methylbenzoic Acid

To a solution of 3-tert-butyl-2-hydroxy-5-[(4-methoxyphenyl)thio]-6-methylbenzoic acid (35 mg, 0.1 mmol) (6) in concentrated acetic acid (2.5 ml) was a 30% solution of hydrogen peroxide (188 μl) added. The reaction mixture was heated at 95° C. for 1 hour resulting in complete conversion. The solvent was evaporated and the crude product was purified by preparative HPLC on a C8-column using a gradient of ammonium acetate buffer/acetonitrile as eluent. Fractions containing the product were pooled and coevaporated from water/acetonitrile, dissolved in water and then freeze-dried to give the title compound as a solid (7 mg, 18% yield).

¹H NMR (400 MHz, CHLOROFORM-d) δ ppm 1.43 (s, 9H) 2.58 (s, 3H) 3.86 (s, 3H) 6.96 (d, 1H) 7.75 (d, 1H) 8.33 (s, 1H); Mass Spectrum (ESI): M−H⁺377.

### Example 49

### 3-tert-butyl-2-hydroxy-6-methyl-5-(1-naphthylsulfonyl)benzoic Acid

The title compound was prepared and isolated as a solid (60 mg, 69% yield) in analogy with example 48, starting from 3-tert-butyl-2-hydroxy-6-methyl-5-(1-naphthylthio)benzoic acid prepared as set forth in example 36.

¹H NMR (400 MHz, CHLOROFORM-d) δ ppm 1.36 (s, 9H) 2.43 (s, 3H) 7.46-7.54 (m, 3H) 7.84-7.90 (m, 1H) 8.01 (d, 1H) 8.24-8.29 (m, 1H) 8.32 (s, 1H) 8.37-8.44 (m, 1H); Mass Spectrum (ESI): M−H⁺397.

### Example 50

### 3-tert-butyl-5-[(2,4-dichlorophenyl)sulfonyl]-2,6-dihydroxybenzoic Acid

The title compound was prepared and isolated as a solid (90 mg, 34% yield) in analogy with example 48, starting from 3-tert-butyl-5-[(2,4-dichlorophenyl)thio]-2,6-dihydroxybenzoic acid prepared as set forth in example 38.

¹H NMR (400 MHz, DMSO-d₆) δ ppm 1.34 (s, 9H) 7.64-7.74 (m, 3H) 8.18 (d, 1H) 16.20-16.39 (m, 2H); Mass Spectrum (ESI): M−H⁺417.

### Example 51

### 3-[(2,4-dichlorophenyl)sulfonyl]-6-hydroxy-5-isopropyl-2-methylbenzoic Acid

The title compound was prepared and isolated as a solid (68 mg, 61% yield) in analogy with example 48, starting from 3-[(2,4-dichlorophenyl)thio]-6-hydroxy-5-isopropyl-2-methylbenzoic acid, prepared as set forth in example 37.

¹H NMR (400 MHz, DMSO-d₆) δ ppm 1.15 (d, 6H) 2.48 (s, 3H) 3.20-3.28 (m, 1H) 7.71 (dd, 1H) 7.77 (d, 1H) 7.87 (s, 1H) 8.17 (d, 1H); Mass Spectrum (ESI): M−H⁺401.

### Example 52

### 3-tert-butyl-5-[(2,4-dichlorophenyl)sulfonyl]-2-hydroxy-6-methylbenzoic Acid

The title compound was prepared and isolated as a solid (45 mg, 76% yield) in analogy with example 48, starting from 3-tert-butyl-5-[(2,4-dichlorophenyl)thio]-2-hydroxy-6-methylbenzoic acid, prepared as set forth in example 42.

¹H NMR (400 MHz, METHANOL-d₄) δ ppm 1.44 (s, 9H) 2.46 (s, 3H) 7.61-7.67 (m, 2H) 8.29 (dd, 1H) 8.35 (s, 1H); Mass spectrum (ESI): 415 M−H⁺

### Example 53

### 3-tert-butyl-5-[(4-chlorophenyl)(ethoxy)methyl]-2-hydroxy-6-methylbenzoic Acid

NaBH₄ (9.5 mg, 0.25 mmol) was added to a solution of 3-tert-butyl-5-(4-chlorobenzoyl)-2-hydroxy-6-methylbenzoic acid (40 mg, 0.12 mmol) in ethanol and stirred over the weekend. Water and HCl were added and extracted with ethyl acetate (3 times). Organic phases were combined, dried over MgSO₄ and solvent evaporated. The crude product was purified first by flash chromatography (acetic acid/ethyl acetate/heptane, 0.01:3:1) and then by preparative HPLC on a C8-column using a gradient of ammonium acetate buffer/acetonitrile as eluent. Fractions containing the product were pooled and freeze-dried to afford the title compound (the yield was not determined).

¹H NMR (400 MHz, CHLOROFORM-d) δ ppm 7.45 (s, 1H), 7.30 (d, 2H), 7.21 (d, 2H), 5.54 (s, 1H), 3.50 (q, 2H), 2.45 (s, 3H), 1.37 (s, 9H), 1.26 (t, 3H).

### Example 54

### 3,5-di-tert-butyl-2,6-dimethoxybenzoic Acid

n-Butyl lithium (1.77 mL, 2.5 M in hexane) was added to a solution of 1,5-di-tert-butyl-2,4-dimethoxybenzene (0.92 g, 3.68 mmol) in anhydrous tetrahydrofuran under N₂ at 10° C. and stirred for 90 minutes. The mixture was allowed to warm up to room temperature and poured into solid CO₂ in ether (10 mL). After 30 minutes, water was added followed by concentrated aqueous HCl. The organic layer was separated and the aqueous phase was extracted twice with ether. Organic phases were combined and extracted twice with NaOH (2 M aqueous solution). Aqueous phases were combined, acidified with concentrated HCl and extracted with DCM. The DCM solution was dried over MgSO₄ and solvent evaporated to afford the title product in 350 mg (32%) yield.

¹H NMR (400 MHz, CHLOROFORM-d) δ ppm 7.39 (s, 1H), 3.88 (s, 6H), 1.39 (s, 18H).

Mass spectrum: m/z M+H 295, M−H 293

### Example 55

### 3-tert-butyl-5-[(2,3-difluorobenzyl)thio]-2-hydroxy-6-methylbenzoic Acid

The title compound was prepared and isolated as a solid (55 mg, 30% yield) in analogy with example 35 using 3,4-difluorobenzenesulfenyl chloride instead of benzyl bromide (the reaction was performed in a Radley carousel).

¹H NMR (400 MHz, DMSO-d₆) δ ppm 1.18 (s, 9H) 2.65 (s, 3H) 3.85 (s, 2H) 6.78-6.86 (m, 2H) 6.99-7.06 (m, 1H) 7.19-7.29 (m, 1H); Mass Spectrum (ESI): M−H⁺365.

3,4-difluorobenzenesulfenyl chloride was synthesized in analogy with 4-methoxybenzenesulfenyl chloride (starting material for Example 35) starting from 3,4-difluorobenzene-thiol and with the exception that sulfuryl chloride was used as chlorinating agent instead of N-chlorosuccinimide.

### Example 56

### 3-tert-butyl-2-hydroxy-6-methyl-5-(pyridin-4-ylthio)benzoic Acid

The title compound was prepared and isolated as a solid (26 mg, 14% yield) in analogy with example 35 using pyridine-4-sulfenyl chloride instead of benzyl bromide.

¹H NMR (400 MHz, CHLOROFORM-d) δ ppm 1.43 (s, 9H) 2.63 (s, 3H) 7.13 (d, 2H) 7.51 (s, 1H) 8.41 (d, 2H); Mass Spectrum (ESI): M−H⁺316.

Pyridine-4-sulfenyl chloride was synthesized in analogy with 4-methoxybenzenesulfenyl chloride (starting material for Example 35) starting from pyridine-4-thiol and with the exception that sulfuryl chloride was used as chlorinating agent instead of N-chloro-succinimide.

### Example 57

### 2-hydroxy-3-isopropyl-6-methyl-5-(1-naphthylsulfonyl)benzoic Acid

The title compound was prepared and isolated as a solid (35 mg, 100% yield) in analogy with example 47 starting from. 2-hydroxy-3-isopropyl-6-methyl-5-(1-naphthylthio)benzoic acid.

¹H NMR (400 MHz, DMSO-d₆) δ ppm 1.21 (d, 6H) 2.48 (s, 3H, overlapping with signal from dimethylsulfoxide) 3.22-3.34 (m, 1H, overlapping with water signal) 7.51-7.61 (m, 2H) 7.69-7.75 (m, 1H) 8.01 (s, 1H) 8.04-8.08 (m, 1H) 8.22-8.28 (m, 2H) 8.32 (d, 1H).

Mass Spectrum (ESI): M−H⁺383.

### Example 58

### 3-tert-butyl-5-{[(5-fluoro-1,3-benzothiazol-2-yl)methyl]thio}-2-hydroxy-6-methylbenzoic Acid

The title compound was prepared and isolated as a solid (15 mg, 18% yield) in analogy with example 43B using 2-(bromomethyl)-5-fluoro-1,3-benzothiazole instead of benzyl bromide (the reaction was performed in a Radley carousel).

¹H NMR (400 MHz, DMSO-d₆) [symbol] ppm 1.09 (s, 9H) 2.58 (s, 3H), 4.43 (s, 2H) 7.18 (s, 1H), 7.31 (dt, 1H), 7.70 (dd, 1H) 8.09 (dd, 1H). Mass Spectrum (ESI): M−H⁺404.

### Example 59

### 3-tert-butyl-2-hydroxy-5-[(3-methoxybenzyl)thio]-6-methylbenzoic Acid

The title compound was prepared and isolated as an oil (99 mg, 55% yield) in analogy with example 43B using 3-methoxybenzyl bromide instead of benzyl bromide (the reaction was performed in a Radley carousel).

¹H NMR (400 MHz, DMSO-d₆) [symbol] ppm 1.20 (s, 9H) 2.68 (s, 3H) 3.63 (s, 3H) 3.74 (s, 2H) 6.53-6.56 (m, 1H) 6.67-6.75 (m, 2H) 6.86 (s, 1H) 7.10-7.16 (m, 1H).

Mass Spectrum (ESI): M−H⁺359.

### Example 60

### 3-tert-butyl-5-[(2-cyanobenzyl)thio]-2-hydroxy-6-methylbenzoic Acid

The title compound was prepared and isolated as a solid (75 mg, 42% yield) in analogy with example 43B using 2-cyanobenzyl bromide instead of benzyl bromide (the reaction was performed in a Radley carousel).

¹H NMR (400 MHz, DMSO-d₆) [symbol] ppm 1.17 (s, 9H) 2.64 (s, 3H) 3.93 (s, 2H) 6.78 (s, 1H) 7.07-7.11 (m, 1H, overlapping with a signal from NH₄OAc) 7.35-7.40 (m, 1H) 7.47-7.52 (m, 1H) 7.70-7.73 (m, 1H). Mass Spectrum (ESI): M−H⁺354.

### Example 61

### 3-tert-butyl-2-hydroxy-6-methyl-5-[(tetrahydro-2H-pyran-2-ylmethyl)thio]benzoic Acid

The title compound was prepared and isolated as a solid (75 mg, 42% yield) in analogy with example 43B using 2-(bromomethyl)tetrahydro-2H-pyran instead of benzyl bromide (the reaction was performed in a Radley carousel).

¹H NMR (400 MHz, DMSO-d₆) [symbol] ppm 1.14-1.19 (m, 1H) 1.32 (s, 9H) 1.34-1.46 (m, 3H) 1.67-1.79 (m, 2H) 2.52-2.59 (m, 1H) 2.62-2.70 (m, 4H) 3.13-3.22 (m, 2H, overlapping with water signal) 3.81-3.88 (m, 1H) 7.19 (s, 1H). Mass Spectrum (ESI): M−H⁺337.

### Example 62

### 3-tert-butyl-2-hydroxy-6-methyl-5-[(pyridin-3-ylmethyl)thio]benzoic Acid

The title compound was prepared and isolated as a solid (29 mg, 18% yield) in analogy with example 43B using 3-(bromomethyl)pyridine instead of benzyl bromide (the reaction was performed in a Radley carousel).

¹H NMR (400 MHz, DMSO-d₆) [symbol] ppm 1.19 (s, 9H) 2.62 (s, 3H) 3.83 (s, 2H) 6.84 (s, 1H) 7.19-7.27 (m, 1H, overlapping with signal from ammonium acetate) 7.38-7.46 (m, 1H) 8.14-8.20 (m, 1H) 8.33-8.41 (m, 1H); Mass Spectrum (ESI): M−H⁺330.

### Example 63

### 3-tert-butyl-2-hydroxy-6-methyl-5-[(pyridin-4-ylmethyl)thio]benzoic Acid

The title compound was prepared and isolated as a solid (16 mg, 10% yield) in analogy with example 43B using 4-(bromomethyl)pyridine instead of benzyl bromide (the reaction was performed in a Radley carousel).

¹H NMR (400 MHz, DMSO-d₆) [symbol] ppm 1.19 (s, 9H) 2.56 (s, 3H) 3.90 (s, 2H) 6.97 (s, 1H) 7.07-7.10 (m, 2H) 8.38-8.43 (m, 2H); Mass Spectrum (ESI): M−H⁺330.

### Example 64

### 3-tert-butyl-2-hydroxy-5-(isobutylthio)-6-methylbenzoic acid

The title compound was prepared and isolated as a solid (78 mg, 53% yield) in analogy with example 43B using isobutylbromide instead of benzyl bromide (the reaction was performed in a Radley carousel).

¹H NMR (400 MHz, DMSO-d₆) [symbol] ppm 0.94 (d, 6H) 1.31 (s, 9H) 1.56-1.68 (m, 1H) 2.50 (m, 2H, overlapping with dimethylsulfoxide-signal) 2.67 (s, 3H) 7.17 (s, 1H); Mass Spectrum (ESI): M−H⁺295.

### Example 65

### 3-tert-butyl-2-hydroxy-6-methyl-5-[(2-phenylethyl)thio]benzoic Acid

The title compound was prepared and isolated as a gum (52 mg, 30% yield) in analogy with example 43B using (2-bromoethyl)benzene instead of benzyl bromide (the reaction was performed in a Radley carousel).

¹H NMR (400 MHz, DMSO-d₆) [symbol] ppm 1.35 (s, 9H) 2.58 (s, 3H) 2.76 (t, 2H) 2.99 (t, 2H) 7.16-7.30 (m, 5H) 7.33 (s, 1H); Mass Spectrum (ESI): M−H⁺343.

### Example 66

### 3-tert-butyl-2-hydroxy-6-methyl-5-{[2-(trifluoromethyl)benzyl]thio}-benzoic Acid

The title compound was prepared and isolated as a solid (88 mg, 44% yield) in analogy with example 43B using 2-trifluoromethylbenzyl bromide instead of benzyl bromide (the reaction was performed in a Radley carousel).

¹H NMR (400 MHz, DMSO-d₆) [symbol] ppm 1.20 (s, 9H) 2.65 (s, 3H) 3.92 (s, 2H) 6.90 (s, 1H) 7.08 (m, 1H, overlapping with signal from ammonium acetate) 7.39-7.51 (m, 2H) 7.63-7.69 (m, 1H); Mass Spectrum (ESI): M−H⁺397.

### Example 67

### 3-tert-butyl-5-[(2,3-difluorobenzyl)sulfonyl]-2-hydroxy-6-methylbenzoic Acid

The title compound was prepared and isolated as a solid (29 mg, 53% yield) in analogy with example 47 starting from 3-tert-butyl-5-[(2,3-difluorobenzyl)thio]-2-hydroxy-6-methylbenzoic Acid

¹H NMR (400 MHz, DMSO-d₆) [symbol] ppm 1.17 (s, 9H) 2.89 (s, 3H) 4.52 (s, 2H) 6.97-7.03 (m, 1H) 7.10-7.18 (m, 1H) 7.33-7.42 (m, 1H); Mass Spectrum (ESI): M−H⁺397.

### Example 68

### 3-tert-butyl-5-[(4-chlorobenzyl)sulfonyl]-2-hydroxy-6-methylbenzoic Acid

The title compound was prepared and isolated as a solid (18 mg, 51% yield) in analogy with example 47 starting from 3-tert-butyl-5-[(4-chlorobenzyl)thio]-2-hydroxy-6-methylbenzoic acid.

¹H NMR (400 MHz, DMSO-d₆) [symbol] ppm 1.17 (s, 9H) 2.88 (s, 3H) 4.43 (s, 2H) 7.04-7.11 (m, 3H) 7.27-7.32 (m, 2H); Mass Spectrum (ESI): M−H⁺395.

### Example 69

### 3-tert-butyl-2-hydroxy-6-methyl-5-[(pyridin-2-ylmethyl)sulfonyl]benzoic Acid

The title compound was prepared and isolated as an oil (5 mg, 25% yield) in analogy with example 47 starting from 3-tert-butyl-2-hydroxy-6-methyl-5-[(pyridin-2-yl-methyl)thio]benzoic acid.

¹H NMR (400 MHz, DMSO-d₆) [symbol] ppm 1.18 (s, 9H) 2.85 (s, 3H) 4.56 (s, 2H) 7.19 (s, 1H) 7.23-7.30 (m, 2H) 7.69-7.75 (m, 1H) 8.38-8.42 (m, 1H); Mass Spectrum (ESI): M−H⁺362.

### Example 70

### 3-tert-butyl-2-hydroxy-6-methyl-5-[(3-methylbenzyl)sulfonyl]benzoic Acid

The title compound was prepared and isolated as a solid (49 mg, 48% yield) in analogy with example 47 starting from 3-tert-butyl-2-hydroxy-6-methyl-5-[(3-methylbenzyl)thio]benzoic acid.

¹H NMR (400 MHz, DMSO-d₆) δ ppm 1.19 (s, 9H) 2.19 (s, 3H) 2.88 (s, 3H) 4.35 (s, 2H) 6.83-6.90 (m, 2H) 7.04-7.16 (m, 2H, overlapping with signal from ammonium acetate) 7.21 (s, 1H, overlapping with signal from ammonium acetate); Mass Spectrum (ESI): M−H⁺375.

### Example 71

### 3-tert-butyl-2-hydroxy-6-methyl-5-[(3-methylbenzyl)thio]benzoic Acid

The title compound was prepared and isolated as a gum (113 mg, 66% yield) in analogy with example 43B using 3-methylbenzyl bromide instead of benzyl bromide (the reaction was performed in a Radley carousel).

Mass Spectrum (ESI): M−H⁺343.

### Example 72

### 3-tert-butyl-2-hydroxy-6-methyl-5-{[2-(trifluoromethyl)benzyl]sulfonyl}-benzoic Acid

The title compound was prepared and isolated as a solid (49 mg, 64% yield) in analogy with example 47 starting from 3-tert-butyl-2-hydroxy-6-methyl-5-{[2-(trifluoromethyl)benzyl]thio}-benzoic acid.

¹H NMR (400 MHz, DMSO-d₆) δ ppm 1.22 (s, 9H) 2.86 (s, 3H) 4.59 (s, 2H) 7.32 (s, 1H) 7.38 (d, 1H) 7.52-7.64 (m, 2H) 7.72 (d, 1H). Mass Spectrum (ESI): M−H⁺429.

### Example 73 and Example 74

### 3-tert-butyl-2-hydroxy-6-methyl-5-(phenylacetyl)benzoic Acid and 3-tert-butyl-2-hydroxy-6-methyl-5-[phenyl(phenylthio)acetyl]benzoic Acid

Phenylacetyl chloride (1.37 mL) was added to a suspension of aluminum trichloride (1.28 g) in 5 mL of dichloroethane and stirred at room temperature for 15 min. The temperature was lowered to −15° C. and a solution of methyl 3-tert-butyl-2-hydroxy-6-methylbenzoate (1.05 g) in 5 mL dichloroethane was added. The reaction was stirred over night while the temperature increased to −7° C. and then partitioned between dichloromethane and 1M hydrochloric acid. The organic layer was washed with 1M hydrochloric acid, water and aqueous sodium hydrogencarbonate, dried, filtered and concentrated. Silica gel chromatography (5% ethyl acetate in heptanes) yielded 3-tert-butyl-2-hydroxy-6-methyl-5-(phenylacetyl)benzoate (1.28 g, 75%). %). Mass Spectrum (ESI): M−H⁺339.5. Sodium thiophenolate (1.09 g) and methyl 3-tert-butyl-2-hydroxy-6-methyl-5-(phenylacetyl)benzoate (234 mg) were added to 3 mL N,N-dimethylformamide and heated to 130° C. under N₂-atmosphere for 4.5 h and then stirring was continued at room temperature over night. Approximately 20 mL acetic acid and 30 mL petroleum ether (175-210° C.) were added and the solvents were evaporated at 70° C. Trituration with petroleum ether gave crystals which were treated with toluene. The formed solids were filtered off and the toluene was evaporated and the residue was purified by preparative HPLC to give two products:

3-tert-butyl-2-hydroxy-6-methyl-5-(phenylacetyl)benzoic acid (45 mg, 20).

¹H NMR (400 MHz, METHANOL-d₄) δ ppm 1.36 (s, 9H), 2.56 (s, 3H), 4.15 (s, 2H), 7.09-7.36 (m, 5H), 7.51 (s, 1H). Mass Spectrum (ESI): M−H⁺325.5 and

3-tert-butyl-2-hydroxy-6-methyl-5-[phenyl(phenylthio)acetyl]benzoic acid (35 mg, 11%).

¹H NMR (400 MHz, METHANOL-d₄) δ ppm 1.27 (s, 9H), 2.45 (s, 3H), 5.79 (s, 1H), 7.13-7.42 (m, 11H), 7.51. Mass Spectrum (ESI): M−H⁺433.5

### Example 75

### 3,5-Di-tert-butyl-2-chloro-6-hydroxybenzoic acid

2,4-di-tert-butyl-5-chlorophenol (3.85 g) and sodium hydroxide were added to 35 mL of dry pyridine and heated to 80° C. until the sodium hydroxide was dissolved. The temperature was increased to 135° C. and about half of the pyridine was distilled from the reaction. The temperature was lowered to 115° C. and CO₂ was bubbeled through the solution for 90 min. The temperature was lowered to 90° C. and the reaction was left over night. The reaction was cooled to room temperature, 30 mL water was added and the solution was transferred to a separatory funnel. 250 mL of each water and toluene was added to the funnel and the pH of the aqueous phase was adjusted to pH 3. After extraction the organic layer was washed with 200 mL water adjusted to pH 8 with 2M aqueous sodium hydroxide, the separated aqueous layer was acidified and washed with ethyl acetate. The organic layer was dried, filtered and evaporated to give a brownish solid. The solid was re-crystallized with acetic acid—water to give the product (0.74 g, 16%).

¹H NMR (400 MHz, DMSO-d₆) δ ppm: 1.34 (s, 9H), 1.42 (s, 9H), 7.30 (s, 1H).

### Example 76

### 3-tert-butyl-5-[(3,4-difluorophenyl)thio]-2-hydroxy-6-methylbenzoic Acid

The title compound was prepared and isolated as a solid (0.61 g mg, 99% yield) in analogy with example 35, using 3,4-difluorobenzenesulfenyl chloride instead of 4-methoxybenzenesulfenyl chloride.

¹H NMR (400 MHz, DMSO-d₆) δ ppm: 1.34 (s, 9H), 2.49 (inside dimethylsulfoxide-peak), 6.75-6.83 (m, 1H), 7.03-7.13 (m, 1H), 7.31-7.42 (m, 1H), 7.47 (s, 1H). Mass Spectrum (ESI): M−H⁺351.

4-(Trifluoromethoxy)benzenesulfenyl chloride was synthesized in analogy with 4-methoxybenzenesulfenyl chloride (starting material for Example 35) starting from 4-(trifluoromethoxy)benzenethiol and with the exception that sulfuryl chloride was used as chlorinating agent instead of N-chlorosuccinimide.

### Example 77

### 3-tert-butyl-5-[(3,4-difluorophenyl)sulfonyl]-2-hydroxy-6-methylbenzoic Acid

The title compound was prepared and isolated as a solid (0.19 g, 35% yield) in analogy with example 48, starting from 3-tert-butyl-5-[(3,4-difluorophenyl)thio]-2-hydroxy-6-methylbenzoic acid.

¹H NMR (400 MHz, DMSO-d₆) δ ppm: 1.40 (s, 9H), 2.37 (s, 3H), 7.62-7.75 (m, 2H), 7.92-8.01 (m, 1H), 8.09 (s, 1H). Mass Spectrum (ESI): M−H⁺383.

### Example 78

### 3-tert-butyl-2-hydroxy-6-methyl-5-[(2,4,5-trichlorophenyl)sulfonyl]benzoic Acid

The title compound was prepared and isolated as a solid (0.19 g, 35% yield) in analogy with example 48, starting from 3-tert-butyl-2-hydroxy-6-methyl-5-[(2,4,5-trichlorophenyl)thio]benzoic acid.

¹H NMR (400 MHz, DMSO-d₆) δ ppm: 1.39 (s, 9H), 2.32 (s, 3H), 8.08 (s, 1H), 8.15 (s, 1H), 8.35 (s, 1H). Mass Spectrum (ESI): M−H⁺449, 451.

Preparation of 3-tert-butyl-2-hydroxy-6-methyl-5-[(2,4,5-trichlorophenyl)thio]benzoic acid is described by Brown et al in Journal of the Chemical Society, Perkin Transactions 1: Organic and Bio-Organic Chemistry (1978), (6), 633-8.

### Example 79

### 3-tert-butyl-2-hydroxy-6-methyl-5-{[4-(trifluoromethoxy)phenyl]sulfonyl}benzoic Acid

The title compound was prepared and isolated as a solid (0.23 g, 37% yield) in analogy with example 48, starting from 3-tert-butyl-2-hydroxy-6-methyl-5-{[4-(trifluoromethoxy)-phenyl]thio}benzoic acid.

¹H NMR (400 MHz, DMSO-d₆) δ ppm: 1.40 (s, 9H), 2.36 (s, 3H), 7.54-7.64 (m, 2H), 7.91-8.00 (m, 2H), 8.10 (s, 1H). Mass Spectrum (ESI): M−H+431.

The starting materials for this compound were synthesized as follows:

### 4-(Trifluoromethoxy)benzenesulfenyl Chloride

The title compound was synthesized in analogy with 4-methoxybenzenesulfenyl chloride chloride (starting material for Example 35) starting from 4-(trifluoromethoxy)benzenethiol and with the exception that sulfuryl chloride was used as chlorinating agent instead of N-chlorosuccinimide.

### 3-tert-Butyl-2-hydroxy-6-methyl-5-{[4-(trifluoromethoxy)phenyl]thio}benzoic Acid

The title compound was prepared and isolated as a solid (0.68 g, 99% yield) in analogy with example 35, using 4-(trifluoromethoxy)benzenesulfenyl chloride instead of 4-methoxybenzenesulfenyl chloride.

¹H NMR (400 MHz, DMSO-d₆) δ ppm: 1.34 (s, 9H), 2.49 inside dimethylsulfoxide-peak), 7.05-7.13 (m, 2H), 7.25-8.33 (m, 2H), 7.48 (s, 1H). Mass Spectrum (ESI): M−H⁺399.

### Example 80

### 3-{[3,5-Bis(trifluoromethyl)phenyl]sulfonyl}-5-tert-butyl-6-hydroxy-2-methylbenzoic Acid

The title compound was prepared and isolated as a solid (9 mg, 8% yield) in analogy with example 48, starting from 3-{[3,5-bis(trifluoromethyl)phenyl]thio}-5-tert-butyl-6-hydroxy-2-methylbenzoic acid.

¹H NMR (400 MHz, DMSO-d₆) δ ppm: 1.41 (s, 9H), 2.40 (s, 3H), 8.12 (s, 1H), 8.38 (apparent s, 2H), 8.54 (s, 1H). Mass Spectrum (ESI): M−H⁺483.

The starting materials for this compound were synthesized as follows:

### 3,5-Bis(trifluoromethyl)benzenesulfenyl Chloride

The title compound was synthesized in analogy with 4-methoxybenzenesulfenyl chloride chloride (starting material for Example 35) starting from 3,5-bis(trifluoromethyl)-benzenethiol and with the exception that sulfuryl chloride was used as chlorinating agent instead of N-chlorosuccinimide.

### 3-{[3,5-bis(trifluoromethyl)phenyl]thio}-5-tert-butyl-6-hydroxy-2-methylbenzoic Acid

The title compound was prepared and isolated as a solid (0.15 g, 15% yield) in analogy with example 35, using 4-(trifluoromethoxy)benzenesulfenyl chloride instead of 4-methoxybenzenesulfenyl chloride.

Mass Spectrum (ESI): M−H⁺451.

### Example 81

### 3-tert-butyl-5-[(2,6-dichlorophenyl)sulfonyl]-2-hydroxy-6-methylbenzoic Acid

The title compound was prepared and isolated as a solid (23 mg, 6% yield) in analogy with example 48, starting from 3-{[3,5-dichlorophenyl]thio}-5-tert-butyl-6-hydroxy-2-methylbenzoic acid. An additional purification by dissolving the compound in hot water and filtering through a 0.45 μm syringe filter was performed on this sample.

¹H NMR (400 MHz, DMSO-d₆) δ ppm: 1.37 (s, 9H), 2.37 (s, 3H), 7.56-7.68 (m, 3H), 8.03 (s, 1H). Mass Spectrum (ESI): M−H⁺415.

The starting materials for this compound were synthesized as follows:

### 2,6-dichlorobenzenesulfenyl Chloride

The title compound was synthesized in analogy with 4-methoxybenzenesulfenyl chloride chloride (starting material for Example 35) starting from 2,6-dichlorobenzenehiol and with is the exception that sulfuryl chloride was used as chlorinating agent instead of N-chlorosuccinimide.

### 3-{[2,6-dichlorophenyl]thio}-5-tert-butyl-6-hydroxy-2-methylbenzoic Acid

The title compound was prepared and isolated as a solid (0.41 g, 90% yield) in analogy with example 35, using 2,6-dichlorobenzenesulfenyl chloride instead of 4-methoxybenzenesulfenyl chloride.

Mass Spectrum (ESI): M−H⁺383.

### Example 82

### 3-tert-butyl-5-[(2,3-dichlorophenyl)sulfonyl]-2-hydroxy-6-methylbenzoic Acid

The title compound was prepared and isolated as a solid (29 mg, 6% yield) in analogy with example 48, starting from 3-{[2,3-dichlorophenyl]thio}-5-tert-butyl-6-hydroxy-2-methylbenzoic acid.

¹H NMR (400 MHz, DMSO-d₆) δ ppm: 1.38 (s, 9H), 2.38 (s, 3H), 7.67 (t, 1H), 7.98 (dd, 1H), 8.06 (s, 1H), 8.21 (dd, 1H). Mass Spectrum (ESI): M−H⁺415.

The starting materials for this compound were synthesized as follows:

### 2,3-Dichlorobenzenesulfenyl Chloride

The title compound was synthesized in analogy with 4-methoxybenzenesulfenyl chloride chloride (starting material for Example 35) starting from 2,3-dichlorobenzenehiol and with the exception that sulfuryl chloride was used as chlorinating agent instead of N-chlorosuccinimide.

### 3-{[2,3-Dichlorophenyl]thio}-5-tert-butyl-6-hydroxy-2-methylbenzoic Acid

The title compound was prepared and isolated as a solid (0.41 g, 90% yield) in analogy with example 35, using 2,3-dichlorobenzenesulfenyl chloride instead of 4-methoxybenzenesulfenyl chloride.

Mass Spectrum (ESI): M−H⁺383.

### Example 83

### 3-tert-butyl-5-[(2-chloro-4-fluorophenyl)sulfonyl]-2-hydroxy-6-methylbenzoic Acid

The title compound was prepared and isolated as a solid (255 mg, 59% yield) in analogy with example 48, starting from. 3-tert-butyl-5-[(2-chloro-4-fluorophenyl)thio]-2-hydroxy-6-methylbenzoic acid

¹H NMR (400 MHz, DMSO-d₆) δ ppm: 1.39 (s, 9H), 2.29 (s, 3H), 7.55 (dt, 1H), 7.68 (dd, 1H), 8.17 (s, 1H), 8.21 (dd, 1H). Mass Spectrum (ESI): M−H⁺399.

The starting materials for this compound were synthesized as follows:

### 2-chloro-4-fluorobenzenesulfenyl Chloride

The title compound was synthesized in analogy with 4-methoxybenzenesulfenyl chloride chloride (starting material for Example 35) starting from 2-chloro-4-fluorobenzenethiol and with the exception that sulfuryl chloride was used as chlorinating agent instead of N-chlorosuccinimide.

### 3-tert-butyl-5-[(2-chloro-4-fluorophenyl)thio]-2-hydroxy-6-methylbenzoic Acid

The title compound was prepared and isolated as a solid (0.41 g, 90% yield) in analogy with example 35, using 2-chloro-4-fluorobenzenesulfenyl chloride instead of 4-methoxybenzenesulfenyl chloride.

¹H NMR (400 MHz, DMSO-d₆) δ ppm: 1.34 (s, 9H), 2.46 (s, 3H), 6.60 (dd, 1H), 7.15 (dt, 1H), 7.46 (s, 1H), 7.53 (dd, 1H). Mass Spectrum (ESI): M−H⁺367.

### Example 84

### 3-tert-butyl-5-[(3-chloro-4-fluorophenyl)sulfonyl]-2-hydroxy-6-methylbenzoic Acid

The title compound was prepared and isolated as a solid (29 mg, 6% yield) in analogy with example 48, starting from 3-tert-butyl-5-[(3-chloro-4-fluorophenyl)thio]-2-hydroxy-6-methylbenzoic acid.

¹H NMR (400 MHz, DMSO-d₆) δ ppm: 1.40 (s, 9H), 2.37 (s, 3H), 7.55 (dt, 1H), 7.84 (ddd, 1H), 8.06 (dd, 1H), 8.10 (s, 1H). Mass Spectrum (ESI): M−H⁺399.

The starting materials for this compound were synthesized as follows:

### 3-Chloro-4-fluoro Benzenesulfenyl Chloride

The title compound was synthesized in analogy with 4-methoxybenzenesulfenyl chloride chloride (starting material for Example 35) starting from 3-chloro-4-fluorobenzenethiol and with the exception that sulfuryl chloride was used as chlorinating agent instead of N-chlorosuccinimide.

### 3-tert-Butyl-5-F(3-chloro-4-fluorophenyl)thiol-2-hydroxy-6-methylbenzoic Acid

The title compound was prepared and isolated as a solid (0.41 g, 90% yield) in analogy with example 35, using 3-chloro-4-fluoro benzenesulfenyl chloride instead of 4-methoxybenzenesulfenyl chloride.

Mass Spectrum (ESI): M−H⁺368.

### Example 85

### 3-tert-butyl-5-[(3,5-dichlorophenyl)sulfonyl]-2-hydroxy-6-methylbenzoic Acid

The title compound was prepared and isolated as a solid (0.23 g, 51% yield) in analogy with example 48, starting from 3-tert-butyl-5-[(3,5-dichlorophenyl)thio]-2-hydroxy-6-methylbenzoic acid.

¹H NMR (400 MHz, DMSO-d₆) δ ppm: 1.41 (s, 9H), 2.37 (s, 3H), 7.83 (d, 2H), 8.01 (t, 1H), 8.11 (s, 1H). Mass Spectrum (ESI): M−H⁺399.

The starting materials for this compound were synthesized as follows:

### 3,5-Dichlorobenzenesulfenyl Chloride

The title compound was synthesized in analogy with 4-methoxybenzenesulfenyl chloride chloride (starting material for Example 35) starting from 3,5-dichlorobenzenethiol and with the exception that sulfuryl chloride was used as chlorinating agent instead of N-chlorosuccinimide.

### 3-tert-Butyl-5-[(3,5-dichlorophenyl)thio]-2-hydroxy-6-methylbenzoic Acid

The title compound was prepared and isolated as a solid (0.41 g, 90% yield) in analogy with example 35, using 3,5-dichlorobenzenesulfenyl chloride instead of 4-methoxybenzenesulfenyl chloride.

Mass Spectrum (ESI): M−H⁺383.

### Example 86

### 3′-tert-butyl-4-hydroxy-5′-methyl-5-pyridin-3-ylbiphenyl-3-carboxylic Acid

Methyl 3′-tert-butyl-4-methoxy-5′-methyl-5-pyridin-3-ylbiphenyl-3-carboxylate (70 mg, 0.18 mmol) was dissolved in dichloromethane and cooled to −78° C. Boron trichloride (1M in dichloromethane, 1.8 mL, 1.8 mmol) was added and the mixture was kept at −78° C. for 2 h. Methanol was added and the solvent was evaporated. The residue was dissolved in dimethyl formamide:water (3:1) (3 mL), lithium hydroxide (100 mg, 4.1 mmol) was added and the mixture was heated to 150° C. for 5 min in a smith synthesizer. Preparative HPLC and freeze drying afforded the product 22.5 mg (35% yield).

¹H NMR (400 MHz, DMSO-d₆) δ ppm 1.32 (s, 9H), 2.36 (s, 3H), 7.15 (s, 1H), 7.28 (s, 1H), 7.40 (s, 1H), 7.47 (dd, J=7.78, 4.77 Hz, 1H), 7.72 (d, J=2.51 Hz, 1H), 8.03 (d, J=2.51 Hz, 1H), 8.11 (dt, J=7.84, 1.98 Hz, 1H), 8.51-8.55 (m, J=4.02 Hz, 1H), 8.86-8.90 (m, 1H). LC-MS: m/z 360 M−1, 362 M+1.

The starting materials for this compound were synthesized as follows:

### 3-Bromo-2-hydroxy-5-iodobenzoic Acid

2-Hydroxy-5-iodobenzoic acid (4 g, 15.15 mmol) in acetic acid (120 mL) was treated with bromine (0.86 mL, 16.6 mmol) in acetic acid (30 mL). The mixture was left at r.t. for 36 h then poured into ice-water and filtrated. The solid was recrystallized from ethanol and water to afford 2.85 g (55% yield) of the product.

¹H NMR (400 MHz, DMSO-d₆) δ ppm, 8.02 (d, J=2.26 Hz, 1H), 8.10 (d, J=2.26 Hz, 1H).

### Methyl 3-bromo-5-iodo-2-methoxybenzoate

3-Bromo-2-hydroxy-5-iodobenzoic acid (2.85 g, 8.3 mmol) was dissolved in dimethyl formamide (50 mL) and potassium carbonate (2.9 g, 20.8 mmol) and methyl iodide(2.28 mL, 20.8 mmol) was added and the mixture was left o.n. The solvent was evaporated and the residue was dissolved in ethyl acetate and water. Organics was collected and evaporated. Chromatography with heptane/ethyl acetate (0-20%) as solvents afforded the product 2.3 g (73% yield).

¹H NMR (400 MHz, DMSO-d₆) δ ppm 3.80 (s, 3H), 3.85 (s, 3H), 7.99 (d, J=2.26 Hz, 1H), 8.21 (d, J=2.26 Hz, 1H).

### Methyl 3′-tert-butyl-4-methoxy-5′-methyl-5-pyridin-3-ylbiphenyl-3-carboxylate

Methyl 3-bromo-5-iodo-2-methoxybenzoate (290 mg, 0.78 mmol), tetrakis(triphenylphosphine)palladium(0) (58 mg, 0.05 mmol) was dissolved in tetrahydrofuran (5 mL). 3-t-butyl-5-methylphenylboronic acid (150 mg, 0.78 mmol) in ethanol (1 mL) was added together with sodium carbonate (2 M in water, 2.5 mL). The mixture was heated to 60° C. for 16 h and then was pyridine-3-boronic acid (98 mg, 0.8 mmol) added followed by additional tetrakis(triphenylphosphine)palladium(0) (11 mg, 0.01 mmol). The mixture was heated to 100° C. o.n. After cooling to rt. and evaporation of the solvent was the product isolated using preparative HPLC to afford 70 mg (23% yield).

¹H NMR (400 MHz, DMSO-d₆) δ ppm 1.32 (s, 9H) 2.37 (s, 3H), 3.47 (s, 3H), 3.90 (s, 3H), 7.23 (s, 1H), 7.35 (s, 1H) 7.47 (s, 1H), 7.50-7.54 (m, 1H), 7.83 (d, J=2.26 Hz, 1H), 7.92 (d, J=2.51 Hz, 1H), 8.04 (dt, J=8.03, 2.01 Hz, 1H), 8.62 (dd, J=4.89, 1.63 Hz, 1H), 8.81 (d, J=1.51 Hz, 1H). LC-MS: m/z 390 M+1.

### Example 87

### 3-(1-Benzofuran-2-yl)-5-tert-butyl-6-hydroxy-2-methylbenzoic Acid

Methyl 3-(1-benzofuran-2-yl)-5-tert-butyl-6-methoxy-2-methylbenzoate was prepared using General procedure 3B for synthesis of biaryls. Benzofuran-2-boronic acid as boronic acid and the product was isolated using 0-15% ethyl acetate in heptane, 89 mg (87% yield).

¹H NMR (400 MHz, CHLOROFORM-d) δ ppm 7.11-7.14 (m, 1H), 7.04-7.07 (m, 1H), 6.75-6.84 (m, 3H), 6.31-6.32 (m, 1H), 3.50 (s, 3H), 3.37 (s, 3H), 1.92 (s, 3H), 0.95 (s, 9H). GC-MS: m/z 353 M+1.

The product was prepared from methyl 3-(1-benzofuran-2-yl)-5-tert-butyl-6-methoxy-2-methylbenzoate using general procedure 2B for removal of protecting groups. Isolated using preparative HPLC afforded 5.5 mg (7% yield).

¹H NMR (400 MHz, DMSO-d₆) δ ppm, 1.38 (s, 9H) 2.60 (s, 3H), 6.83 (s, 1H), 7.20-7.27 (m, 2H), 7.36-7.39 (m, 1H), 7.55-7.58 (m, 1H), 7.59-7.62 (m, 1H).

LC-MS: m/z 323 M−1.

### Example 88

### 3-tert-butyl-5-(1,1-dioxido-1-benzothien-2-yl)-2-hydroxy-6-methylbenzoic Acid

3-(1-benzothien-2-yl)-5-tert-butyl-6-hydroxy-2-methylbenzoic acid (80 mg, 0.235 mmol) was dissolved in acetic acid (5 mL) and hydrogen peroxide (0.48 mL, 4.7 mmol) was added in 3 portions. The mixture was heated to 90° C. for 1 h, cooled to rt. and evaporated. The product was isolated using preparative HPLC to afford 49 mg (56% yield).

¹H NMR (400 MHz, DMSO-d₆) δ ppm 1.35 (s, 9H), 2.48 (s, 3H), 7.41 (d, J=3.26 Hz, 2H), 7.58-7.62 (m, 2H), 7.68-7.73 (m, 1H), 7.88 (d, J=8.03 Hz, 1H).

LC-MS: m/z 371 M−1.

The starting material for this compound was synthesized as follows:

### Methyl 3-(1-benzothien-2-yl)-5-tert-butyl-6-methoxy-2-methylbenzoate

The product was prepared using General procedure 3B for synthesis of biaryls. Benzothiophene-2-boronic acid as boronic acid and the product was isolated using 0-10% ethyl acetate in heptane, 44 mg (41% yield).

¹H NMR (400 MHz, CHLOROFORM-d) δ ppm 1.41 (s, 9H) 2.31 (s, 3H), 3.86 (s, 3H), 3.99 (s, 3H), 7.19 (s, 1H), 7.32-7.41 (m, 2H), 7.45 (s, 1H), 7.78-7.82 (m, 1H), 7.83-7.87 (m, 1H). GC-MS: m/z 369 M+1.

### 3-(1-Benzothien-2-yl)-5-tert-butyl-6-hydroxy-2-methylbenzoic Acid

The product was prepared from methyl 3-(1-benzothien-2-yl)-5-tert-butyl-6-methoxy-2-methylbenzoate using general procedure 2B for removal of protecting groups. Isolated using preparative HPLC afforded 15 mg (37% yield).

¹H NMR (400 MHz, DMSO-d₆) δ ppm 1.37 (s, 9H) 2.48 (s, 3H), 7.30 (d, J=6.02 Hz, 2H), 7.32-7.41 (m, 2H), 7.83 (d, J=7.28 Hz, 1H), 7.94 (d, J=7.78 Hz, 1H).

LC-MS: m/z 339 M−1.

### Example 89

### 5-tert-butyl-3′,4′-dichloro-4-hydroxy-2-methylbiphenyl-3-carboxylic Acid

Methyl 3-bromo-5-tert-butyl-6-methoxy-2-methylbenzoate (92 mg, 0.29 mmol) and aqueous 2.0 M sodium carbonate (292 μL, 0.58 mmol) were mixed in anhydrous toluene (2 mL) in a microwave vial. 3,4-dichlorophenylboronic acid (72 mg, 0.38 mmol) and tetrakis-(triphenylphosphine)palladium(0) (17 mg, 0.015 mmol) were added, the vial was capped and purged with argon and the reaction was heated in a microwave at 90° C. for 1 hour. Saturated aqueous sodium chloride was added and the product was extracted with ethyl acetate, the organic phase was dried (MgSO₄), filtered and stripped. The crude product was purified by preparative HPLC. The fractions containing product were pooled and the above described aqueous work-up was repeated to give 66 mg (59% yield) of methyl 5-tert-butyl-3′,4′-dichloro-4-methoxy-2-methylbiphenyl-3-carboxylate. MS m/z 381, 383 [M+H]⁺. The product from the first step (60 mg, 0.16 mmol) was dissolved in anhydrous dichloromethane (2 mL) under argon atmosphere and the solution was cooled to −78° C. in a dry-ice/aceton bath. A 1.0 M dichloromethane solution of boron trichloride (1.4 mL, 9 eq.) was added dropwise during 5 minutes and the reaction mixture was stirred at room temperature for 1 hour. Methanol (2 mL) was added carefully and the mixture was stirred until no more gas was evolved. The solvent was evaporated and lithium hydroxide monohydrate (126 mg, 3.0 mmol) followed by a 3:1 mixture of N,N-dimethyl formamide/water (2 mL) were added. The reaction was heated in a microwave at 150° C. for 5 minutes. The mixture was neutralized with a few drops of concentrated hydrochloric acid, the solvent was evaporated and the crude product was purified by preparative HPLC to give 21 mg (38% yield) of the title compound.

¹H-NMR (DMSO-d₆): δ ppm 1.34 (s, 9H), 2.34 (s, 3H), 6.93 (s, 1H), 7.23 (dd, 1H) overlapping with 7.35-7.00 (bs, 1H), 7.47 (d, 1H), 7.61 (d, 1H). MS m/z 351, 353 [M−H]⁻.

### Example 90

### 5-tert-butyl-2′,4′-dichloro-4-hydroxy-2-methylbiphenyl-3-carboxylic Acid

Methyl 5-tert-butyl-2′,4′-dichloro-4-methoxy-2-methylbiphenyl-3-carboxylate was prepared by the procedure described in example 89 except that 1 hour reaction time at 100° C. in the microwave was required. 2,4-dichlorophenyl boronic acid was used as boronic acid to give 63 mg (57% yield) of the protected compound. MS m/z 381, 383 [M+H]⁺.

The title compound was prepared by the procedure described in example 89 starting from methyl 5-tert-butyl-2′,4′-dichloro-4-methoxy-2-methylbiphenyl-3-carboxylate (1.5 hour reaction time was required in the first deprotective step) to give 31 mg (60% yield) of the title compound.

¹H-NMR (DMSO-d₆): δ ppm, 1.32 (s, 9H), 2.21 (s, 3H), 6.71 (s, 1H), 7.25 (dd, J=8.28 Hz, 1H), 7.41 (dd, J=8.16, 2.13 Hz, 1H), 7.62 (d, J=2.26 Hz, 1H). MS m/z 351, 353 [M−H]⁻.

### Example 91

### 5-tert-butyl-4-hydroxy-2-methyl-4′-morpholin-4-ylbiphenyl-3-carboxylic Acid

Procedure 3B was applied. Methyl 3-bromo-5-tert-butyl-6-methoxy-2-methylbenzoate (0.10 g, 0.32 mmol) and (4-morpholin-4-ylphenyl)boronic acid (0.13 g, 0.64 mmol) gave methyl 5-tert-butyl-4-methoxy-2-methyl-4′-morpholin-4-ylbiphenyl-3-carboxylate (52 mg, 41%). Procedure 2B gave the title compound (10 mg, 21%).

¹H NMR (400 MHz, METHANOL-d₄) δ ppm 1.39 (s, 9H), 2.36 (s, 3H), 3.16 (m, 4H), 3.86 (m, 4H), 6.99 (m, 2H), 7.04 (s, 1H), 7.14 (m, 2H). MS (M+H⁺) 370.

### Example 92

### 3-tert-butyl-2-hydroxy-6-methyl-5-(1-naphthyl)benzoic Acid

Procedure 3B was applied. Methyl 3-bromo-5-tert-butyl-6-methoxy-2-methylbenzoate (0.10 g, 0.32 mmol) and 1-naphthylboronic acid (0.11 g, 0.64 mmol) gave methyl 3-tert-butyl-2-methoxy-6-methyl-5-(1-naphthyl)benzoate (0.11 g, 93%). Procedure 2A gave the title compound (20 mg, 20%).

¹H NMR (400 MHz, METHANOL-d₄) δ ppm 1.40 (s, 9H), 2.14 (s, 3H), 7.04 (s, 1H), 7.27 (m, 1H), 7.36 (m, 1H), 7.41-7.52 (m, 3H), 7.83 (d, J=8.5 Hz, 1H), 7.88 (d, J=8.5 Hz, 1H). Mass Spectrum (M+H⁺) 335.

### Example 93

### 5-tert-butyl-3′-cyano-4-hydroxy-2-methylbiphenyl-3-carboxylic Acid

Procedure 3B was applied. Methyl 3-bromo-5-tert-butyl-6-methoxy-2-methylbenzoate (0.10 g, 0.32 mmol) and 3-cyanoboronic acid (94 mg, 0.64 mmol) gave methyl 5-tert-butyl-3′-cyano-4-methoxy-2-methylbiphenyl-3-carboxylate (45 mg, 42%). Procedure 2A gave the title compound (33 mg, 82%).

¹H NMR (400 MHz, METHANOL-d₄) δ ppm 1.40 (s, 9H), 2.36 (s, 3H), 7.00 (s, 1H), 7.54-7.61 (m, 3H), 7.65 (m, 1H). Mass Spectrum (M−H⁺) 308.

### Example 94

### 5-tert-butyl-4-hydroxy-2-methyl-3′,5′-bis(trifluoromethyl)biphenyl-3-carboxylic Acid

Procedure 3B was applied. Methyl 3-bromo-5-tert-butyl-6-methoxy-2-methylbenzoate (0.10 g, 0.32 mmol) and [3,5-bis(trifluoromethyl)phenyl]boronic acid (0.17 g, 0.64 mmol) gave methyl 5-tert-butyl-4-methoxy-2-methyl-3′,5′-bis(trifluoromethyl)biphenyl-3-carboxylate (0.14 mg, quant.). Procedure 2A gave the title compound (71 mg, 52%).

¹H NMR (400 MHz, METHANOL-d₄) δ ppm 1.40 (s, 9H), 2.37 (s, 3H), 7.03 (s, 1H), 7.81 (s, 2H), 7.88 (s, 1H). Mass Spectrum (M−H⁺) 419.

### Example 95

### 3-tert-butyl-2-hydroxy-6-methyl-5-(2-naphthyl)benzoic Acid

Procedure 3B was applied. Methyl 3-bromo-5-tert-butyl-6-methoxy-2-methylbenzoate (0.10 g, 0.32 mmol) and 2-naphthylboronic acid (0.11 g, 0.64 mmol) gave methyl 3-tert-butyl-2-methoxy-6-methyl-5-(2-naphthyl)benzoate (0.11 g, 95%). Procedure 2A gave the title compound (59 mg, 55%).

¹H NMR (400 MHz, METHANOL-d₄) δ ppm 1.42 (s, 9H), 2.41 (s, 3H), 7.15 (s, 1H), 7.40 (dd, J=8.5, 2 Hz, 1H), 7.46 (m, 2H), 7.69 (m, 1H), 7.82-7.88 (m, 3H). Mass Spectrum (M−H⁺) 333.

### Example 96

### 3-tert-butyl-2-hydroxy-5-isoquinolin-4-yl-6-methylbenzoic Acid

Procedure 3B was applied. Methyl 3-bromo-5-tert-butyl-6-methoxy-2-methylbenzoate (0.10 g, 0.32 mmol) and isoquinolin-4-ylboronic acid (65 mg, 0.38 mmol) gave methyl 3-tert-butyl-5-isoquinolin-4-yl-2-methoxy-6-methylbenzoate (10 mg, 9%). Procedure 2B gave the title compound (2 mg, 22%).

¹H NMR (400 MHz, METHANOL-d₄) δ ppm 1.41 (s, 9H), 2.18 (s, 3H), 7.14 (s, 1H), 7.53 (d, J=8 Hz, 1H), 7.74 (m, 2H), 8.18 (d, J=8 Hz, 1H), 8.26 (s, 1H), 9.25 (s, 1H). Mass Spectrum (M+H⁺) 336.

### Example 97

### 3-tert-butyl-2-hydroxy-6-methyl-5-quinolin-3-ylbenzoic Acid

Procedure 3B was applied. Methyl 3-bromo-5-tert-butyl-6-methoxy-2-methylbenzoate (0.10 g, 0.32 mmol) and quinolin-3-ylboronic acid (65 mg, 0.38 mmol) gave methyl 3-tert-butyl-2-methoxy-6-methyl-5-quinolin-3-ylbenzoate (16 mg, 13%). Procedure 2B gave the title compound (5 mg, 33%).

¹H NMR (400 MHz, METHANOL-d₄) δ ppm 1.43 (s, 9H), 2.44 (s, 3H), 7.22 (s, 1H), 7.65 (m, 1H), 7.79 (m, 1H), 7.99 (d, J=8 Hz, 1H), 8.07 (d, J=8.5 Hz, 1H), 8.24 (d, J=2 Hz, 1H), 8.77 (d, J=2 Hz, 1H). Mass Spectrum (M+H⁺) 336.

### Example 98

### 3-tert-butyl-2-hydroxy-6-methyl-5-quinolin-8-ylbenzoic Acid

Procedure 3B was applied. Methyl 3-bromo-5-tert-butyl-6-methoxy-2-methylbenzoate (0.10 g, 0.32 mmol) and quinolin-8-ylboronic acid (65 mg, 0.38 mmol) gave methyl 3-tert-butyl-2-methoxy-6-methyl-5-quinolin-8-ylbenzoate (50 mg, 43%). Procedure 2B gave the title compound (14 mg, 32%).

¹H NMR (400 MHz, METHANOL-d₄) δ ppm 1.40 (s, 9H), 2.15 (s, 3H), 7.10 (s, 1H), 7.50 (dd, J=8.5, 4 Hz, 1H), 7.57 (dd, J=7, 1.5 Hz, 1H), 7.64 (m, 1H), 7.93 (dd, J=8, 1.5 Hz, 1H), 8.39 (dd, J=8.5, 2 Hz, 1H), 8.72 (dd, J=4, 2 Hz, 1H). Mass Spectrum (M+H⁺) 336.

### Example 99

### 3-tert-butyl-2-hydroxy-6-methyl-5-quinolin-6-ylbenzoic Acid

Procedure 3B was applied. Methyl 3-bromo-5-tert-butyl-6-methoxy-2-methylbenzoate (0.10 g, 0.32 mmol) and quinolin-6-ylboronic acid (65 mg, 0.38 mmol) gave methyl 3-tert-butyl-2-methoxy-6-methyl-5-quinolin-6-ylbenzoate (60 mg, 51%). Procedure 2B gave the title compound (30 mg, 55%).

¹H NMR (400 MHz, METHANOL-d₄) δ ppm 1.42 (s, 9H), 2.42 (s, 3H), 7.25 (s, 1H), 7.57 (dd, J=8.5, 4 Hz, 1H), 7.72 (dd, J=9, 2 Hz, 1H), 7.82 (d, J=2 Hz, 1H), 8.06 (d, J=9 Hz, 1H), 8.40 (d, J=8.5 Hz, 1H), 8.85 (dd, J=4, 1.5 Hz, 1H). Mass Spectrum (M−H⁺) 334.

### Example 100

### 3-tert-butyl-2-hydroxy-6-methyl-5-quinolin-5-ylbenzoic Acid

Procedure 3B was applied. Methyl 3-bromo-5-tert-butyl-6-methoxy-2-methylbenzoate (0.10 g, 0.32 mmol) and quinolin-5-ylboronic acid (0.11 g, 0.64 mmol) gave methyl 3-tert-butyl-2-methoxy-6-methyl-5-quinolin-5-ylbenzoate (27 mg, 23%). Procedure 2B gave the title compound (10 mg, 42%).

¹H NMR (400 MHz, METHANOL-d₄) δ ppm 1.41 (s, 9H), 2.14 (s, 3H), 7.19 (s, 1H), 7.48 (m, 2H), 7.84 (m, 1H), 7.91 (d, J=8.5 Hz, 1H), 8.06 (d, J=8.5 Hz, 1H), 8.85 (dd, J=4, 1.5 Hz, 1H). Mass Spectrum (M−H⁺) 334.

### Example 101

### 4′-hydroxy-6′-methoxy-1,1′:3′,1″-terphenyl-5′-carboxylic Acid

Methyl 2,6-dihydroxybenzoate (84 mg, 0.5 mmol) was treated with N-bromosuccinimide (0.18 g, 1.0 mmol) in MeCN (4 mL) at 25° C. for 12 h. The solvent was evaporated and the is residue extracted with ether. Filtration and evaporation gave methyl 3,5-dibromo-2,6-dihydroxybenzoate (0.17 g, 0.5 mmol). This product and potassium carbonate (0.21 g, 1.5 mmol) was dissolved in N,N-dimethylformamide (5 mL) and treated with methyl iodide (93 μL, 1.5 mmol). Aqueous workup with ethyl acetate/water, was followed by chromatography on silica (0 to 100% ethyl acetate in heptane) to give methyl 3,5-dibromo-2,6-dimethoxybenzoate (90 mg, 50%).

Procedure 3B was applied. Methyl 3,5-dibromo-2,6-dimethoxybenzoate (90 mg, 0.25 mmol) and phenylboronic acid (67 mg, 0.55 mmol) was reacted 2 days to give methyl 4′,6′-dimethoxy-1,1′:3′,1″-terphenyl-5′-carboxylate (50 mg, 57%). Procedure 2A selectively removed one of the two methyl ethers and gave the title compound (22 mg, 49%).

¹H NMR (400 MHz, METHANOL-d₄) δ ppm 3.51 (s, 3H), 4.85 (s), 7.28 (m, 2H), 7.31 (s, 1H), 7.38 (m, 4H), 7.55 (m, 4H). Mass Spectrum (M+H⁺) 321.

### Example 102

### 4,4″-difluoro-4′-hydroxy-1,1′:3′,1″-terphenyl-5′-carboxylic Acid

3,5-dibromo-2-hydroxybenzoic acid (2.0 g, 5 mmol) and potassium carbonate (2.0 g, 15 mmol) was dissolved in N,N-dimethylformamide (25 mL) and treated with methyliodide (0.94 mL, 15 mmol) and stirred at 25° C. for two days. Aqueous workup with ethyl acetate/water, was followed by chromatography on silica (0 to 100% ethyl acetate in heptane) to give methyl 3,5-dibromo-2-methoxybenzoate (1.8 g, quant.).

Procedure 3B was applied. Methyl 3,5-dibromo-2-methoxybenzoate (0.10 g, 0.31 mmol) and 4-fluoro-phenylboronic acid (95 mg, 0.68 mmol) gave methyl 4,4″-difluoro-4′-methoxy-1,1′:3′,1″-terphenyl-5′-carboxylate (0.10 g, 96%). Procedure 2A gave the title compound (69 mg, 70%).

¹H NMR (400 MHz, METHANOL-d₄) δ ppm 4.89 (s), 7.13 (m, 4H), 7.56 (d, J=2.5 Hz, 1H), 7.64 (m, 4H), 8.13 (d, J=2.5 Hz, 1H). Mass Spectrum (M−H⁺) 325.

### Example 103

### 3-tert-butyl-4′-hydroxy-5-methyl-1,1′:3′,1″-terphenyl-5′-carboxylic Acid

5-Chloro-2-hydroxybenzoic acid (86 mg, 0.5 mmol) was treated with N-bromosuccinimide (89 mg, 0.5 mmol) in carbon disulphide (3 mL) at 25° C. After 12 h another portion of N-bromosuccinimide (45 mg, 0.25 mmol) was added. After a further 2 h the solvent was evaporated to give crude 3-bromo-5-chloro-2-hydroxybenzoic acid (˜0.5 mmol). This product and potassium carbonate (0.21 g, 1.5 mmol) was dissolved in N,N-dimethylformamide (5 mL) and treated with methyl iodide (93 μL, 1.5 mmol) and stirred at 25° C. for 12 h. Aqueous workup with ethyl acetate/water, was followed by chromatography on silica (0 to 100% ethyl acetate in heptane) to give methyl 3-bromo-5-chloro-2-methoxybenzoate (90 mg, 64%).

Methyl 3-bromo-5-chloro-2-methoxybenzoate (45 mg, 0.16 mmol) and phenylboronic acid (20 mg, 0.16 mmol) was reacted according to procedure 3 but potassium carbonate (0.16 mL, 2M (aq), 0.32 mmol) was used as base and Pd₂(dppf)₂Cl₂ (6 mg, 5%) as catalyst. The reaction was complete after 2 h at 100° C. and purification gave methyl 5-chloro-2-methoxybiphenyl-3-carboxylate (20 mg, 72 μmol). Then, procedure 3B was applied and the product reacted with (3-tert-butyl-5-methylphenyl)boronic acid (28 mg, 0.14 mmol) to give methyl 3-tert-butyl-4′-methoxy-5-methyl-1,1′:3′,1″-terphenyl-5′-carboxylate (44 mg, quant.). Procedure 2A gave the title compound (10 mg, 25%).

¹H NMR (400 MHz, METHANOL-d₄) δ ppm 1.36 (s, 9H), 2.39 (s, 3H), 7.16 (s, 1H), 7.24 (s, 1H), 7.31 (m, 1H), 7.38-7.45 (m, 3H), 7.59 (d, J=2.5 Hz, 1H), 7.64 (m, 2H), 8.15 (d, J=2.5 Hz, 1H). Mass Spectrum (M+H⁺) 361.

### Example 104

### 2,6-dihydroxy-3,5-diisopropylbenzoic Acid

4,6-diisopropylbenzene-1,3-diol (194 mg, 1 mmol) and potassium bicarbonate (1 g, 10 mmol) were dissolved in N,N-dimethylformamide (5 mL) and the reaction was heated at 135° C. for 12 h under CO₂ (g) flow. The crude product was concentrated in vacuo and purified by preparative HPLC, giving 142 mg of pure material, 73% yield.

¹H NMR (400 MHz, MeOD) δ ppm 1.16 (d, 12H), 3.11-3.27 (m, 2H), 5.48 (s, 2H), 6.99 (s, 1H). Mass Spectrum (M−H⁺) 237.

### INTERMEDIATES

### Example A

### 3-tert-Butyl-2-hydroxy-5-iodo-6-methylbenzoic Acid

3-tert-Butyl-2-hydroxy-6-methylbenzoic acid (400 mg, 1.92 mmol) was dissolved in anhydrous dimethylformamide (4 ml) and put under nitrogen atmosphere. Iodine monochloride (374 mg, 2.30 mmol) was added and the reaction was stirred at room temperature for 15 minutes and then heated at 80° C. for 2 h. The reaction mixture was poured onto ice-water and the solid was isolated by filtration. The solid was dissolved in dimethylsulfoxide and methanol and filtered through a 6 ml C18EC-SPE. The product was eluted with methanol. The eluate was concentrated by evaporation to 4.5 ml. 1.5 ml was purified by prep-HPLC to give a white solid (0.10 g, 97% pure according to LC-UV). The remaining 3 ml was poured onto water and the precipitate was collected by filtration and dried in a vacuum deccicator with Sicapent to give a beige solid (0.25 g, 83% pure according to LC-UV). Total yield 0.35 g, 55%.

¹H NMR (400 MHz, METHANOL-d₄) δ ppm 1.36 (s, 9H) 2.64 (s, 3H) 7.78 (s, 1H)

¹³C NMR (101 MHz, METHANOL-d₄) δ ppm 29.2, 29.7, 35.5, 91.9, 116.5, 138.7, 141.5, 142.2, 162.3, 174.6. Mass Spectrum (ESI) 333 (M−H⁺)⁻

### Example B

### Methyl 3-bromo-5-tert-butyl-6-methoxy-2-methylbenzoate

3-Bromo-5-tert-butyl-6-hydroxy-2-methylbenzoic acid (3.0 g, 10 mmol) and potassium carbonate (2.9 g, 21 mmol) was dissolved in dimethylformamide (25 mL) and methyl iodide (1.6 mL, 26 mmol) was added. The reaction mixture was stirred at 25° C. After 12 h, additional potassium carbonate (0.69 g, 5.0 mmol) and methyl iodide (0.31 mL, 5.0 mmol) was added. After two days, evaporation in vacuo and extraction with ethyl acetate/water, was followed by chromatography on silica (0 to 30% ethyl acetate in heptane) to give the product (2.8 g, 88%).

¹H NMR (400 MHz, CHLOROFORM-d) δ ppm 1.36 (s, 9H), 2.28 (s, 3H), 3.78 (s, 3H), 3.95 (s, 3H), 7.51 (s, 1H).

### Example C

### Methyl 3-tert-butyl-5-iodo-2-methoxy-6-methylbenzoate

Procedure as for methyl 3-bromo-5-tert-butyl-6-methoxy-2-methylbenzoate (Example B). 3-tert-Butyl-2-hydroxy-5-iodo-6-methylbenzoic acid (3.6 g, 10.7 mmol) gave after chromatography on silica (0 to 10% ethyl acetate in heptane) the product (2.6 g, 67%).

¹H NMR (400 MHz, CHLOROFORM-d) δ ppm 1.35 (s, 9H), 2.32 (s, 3H), 3.78 (s, 3H), 3.94 (s, 3H), 7.77 (s, 1H).

### Pharmacology

### In Vitro Model

hGlyRα1Electrophysiology

Transfected L(tk)⁻ cells stably expressing human GlyRα1 homomers were incubated at 37° C. (5% CO₂) in tissue flasks (Costar) containing Modified Eagle Medium+Earles+L-glutamin (MEM; GibcoBRL) supplemented with 10% heat-inactivated fetal calf serum, 100 IU/ml Penicillin/Streptomycin (GibcoBRL). Cells were split twice weekly, using mild trypsination. The cells were split and seeded in 50 mm cell culture dishes 24-48 h prior to the experiment.

Glycine receptor-mediated whole-cell currents were recorded under voltage-clamp conditions. Borosilicate glass pipettes (GC 150-10, Clark Electromedical Instruments) were used. The cell culture dish was fitted with an inset giving a recording chamber volume of 0.6 ml. The chamber was continuously perfused with extracellular solution (see below) at 1.5 ml/min. Test compounds were delivered by a DAD-12 superfusion system (Adams & List Associates, Ltd, Westbury, N.Y.; USA). The signals were recorded using an Axopatch 200A amplifier, a Digidata interface and the pClamp software (all from Axon Instruments, Foster City, Calif.). No series resistance compensation was used. All experiments were performed at room temperature.

Extracellular solution contained (in mM): NaCl 137, KCl 5.0, CaCl₂ 1.0, MgCl₂ 1.2, HEPES 10, glucose 10, pH adjusted to 7.4 with NaOH. Intracellular solution contained (in mM): KCl 140, NaCl 3.0, MgCl₂ 1.2, EGTA 1.0, HEPES 10, pH adjusted to 7.2 with KOH.

Glycine (Sigma) stock solution was prepared fresh each day in extracellular solution. The test compounds were dissolved in dimethylsulfoxide to a concentration of 20 mM and diluted in the extracellular solution to the final concentration. The concentration-response curve was obtained by first applying a 40 μM control concentration of glycine for 10 seconds. The lowest concentration of test compound was subsequently applied for 10 seconds alone, then co-applied with 40 μM glycine for 10 seconds. This sequence was repeated with 4 concentrations of test compound on each cell. There was no washout of compound between concentrations.

Raw data was analysed using the pClamp software. The peak currents were measured and normalized to the control glycine current. Concentration-response relationships were plotted using Origin 6.1 (OriginLab® Corporation, Northampton, Mass.).

Typical IC₅₀ values for the compounds of the present invention are in the range of about 0.1 to about 1,000,000 nM. Other values for IC₅₀ are in the range of about 1 to about 100,000 nM. Further values for IC₅₀ are in the range of about 10 nM to about 30,000 nM

### In Vivo Model

### Freund's Complete Adjuvant (FCA) Induced Arthritis in Rat

### Animals

Male Sprague Dawley rats (B&K Universal AB, Uppsala, Sweden) weighing 150 to 300 g at the time of FCA injection are being used. Rats are held up to 6 in transparent Macrolon® IV cages with wood shavings as bedding. Holding and study areas have automatic control of the light cycle (12:12 hr), the temperature (21±2° C.) and the humidity (40 to 80%).

### Experimental Procedure

Under isoflurane anesthesia, 40 μl of FCA (1 mg/mL) is injected into the left tibio-tarsal (ankle) joint from the dorsal side of the rats. The injection causes a localized inflammation and the animals display decreased weight bearing on and guarding of the limb. The animals are allowed to recover in their home cage for 48 hours following the injection of FCA before any experiment is performed. Forty-eight hours after induction of arthritis and at measurement times depending on the kinetics of the test compound, the rats are placed in a Plexiglas chamber and videotaped for 5 min from underneath. Subsequently, the weight the rats were willing to put on the injected paw are scored as 0: normal paw position, 1: the paw is used during walking, but the toes are kept together, 2: pronounced limping, 3: the paw does not contact the floor.

### Administration of the Substance

Rats are injected orally, subcutaneously or intraperitonealy depending on kinetic profile of the test substance. The time between administration and videotaping is also dependant on the kinetics of the test compound.

### Neuropathic Pain Model—Modified Chung Model

### Animals

Male Sprague-Dawley (Hsd: SD) rats (Charles River, St Constant, Canada) weighing approximately 100-150 g are ordered for surgery. Rats are housed in groups of 7-9 in a temperature controlled room (22±1.5° C., 30-80% humidity, 12 h light/dark cycle). Rats are acclimatized in the animal facility for at least one-day prior to use. Experiments are performed during the light phase of the cycle, rooms are illuminated at 300 lux intensity. Animals have food and water ad libitum.

Experimental Procedures—Modified Spinal Nerve Ligation Model (also Called Modified SNL or Modified Chung Model) (Chung et al. 2004)

Under ketamine and xylazine anesthesia, a dorsal mid-line incision are made approximately from the lower lumbar (L3) level to sacral (S2) level allowing exposure of the muscles. The left paraspinal muscles are isolated and removed from the L4 spinous level to the sacrum S1 level. The bone, L6 transverses process, is then removed to allow easy access to the L5 spinal nerve. The left L5 and L6 spinal nerves are carefully isolated and tightly ligated with 4-0 silk threads whereas L4 is “tickled” about 10 times using glass hook. The incision is closed in layers using an appropriate suture material. Rats are allowed to recuperate until post-operative day 10 at which time testing can begin.

### Test Procedure

Rats are placed on a grid floor, and are covered by a reversed small animal cage. In order to determine the rat's threshold (measured in g) to a tactile mechanical stimulus baseline measurements are determined by touching the treated paw with a series of monofilaments of incremental stiffness in the “up/down” method (Chaplan et al. (1994)).

After determination of the rat's threshold to the tactile mechanical stimulus, rats are randomized in homogeneous groups before experiments are started. Rats having mechanical threshold higher than 5 g are exclude from the study.

### Administration of the Substance

Rats are injected orally, subcutaneously or intraperitonealy depending on kinetic profile of the test substance. The time between administration and videotaping is also dependant on the kinetics of the test compound.

### List of Abbreviations

HEPES=4-(2-Hydroxyethyl)piperazine-1-ethanesulfonic acid
EGTA=Ethylene glycol-bis(2-aminoethylether)-N,N,N′,N′-tetraacetic acid
THF=tetrahydrofuran

1-41. (canceled)

42. A compound of formula I, or a pharmaceutically acceptable salt thereof 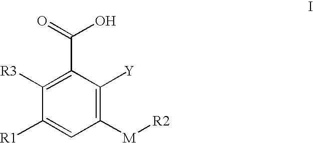
wherein;
Y is selected from hydrogen, —OH, halo, —OC₁₋₆alkyl, or —C₁₋₆alkyl, the two latter optionally substituted with halo, —CN, —OH, —CF₃, —NH₂;
R1 is selected from —C₃₋₆cycloalkyl, heterocycloalkyl, aryl, alkylaryl, heteroaryl, or —C₃₋₆-alkyl, optionally substituted with halo, —CN, —OH, —CF₃, —OCF₃, —NH₂, or —CONH₂;
M is selected from —C(O)—, —C(H₂)—, —CH(OR^{a})—, —N(OH)—, —N(R^{a})—, —S(O)ᵣ—, heteroaryl or a bond;
wherein R^{a} is hydrogen or C₁₋₆alkyl and r is 0, 1 or 2;
R2 is either selected from hydrogen, halo, —CN, or is a group D selected from —C₁₋₆alkyl, C₃₋₆cycloalkyl, heterocycloalkyl, —N(CH₃)₂, aryl, alkylaryl, heteroaryl, or a heterocyclic group;
where D is optionally substituted with one or more substituents G selected from halo, —NO₂, —CN, —OH, —CF₃, —OCF₃, —NH₂, —CONH₂, —COOH, aryl, heteroaryl, heterocyclic groups, —C₁₋₆alkyl, —C₁₋₆alkoxy, heterocycloalkyl, or C₁₋₆alkylcarboxylate;
where D may optionally be connected to G by a linker group L selected from —C(O)—, —S—, or —S(O₂)—;
and G, if substitutable, is optionally further substituted with one or more substituents selected from halo, —NO₂, —CN, —OH, —CH₃, —OCH₃, —CF₃, —OCF₃, —NH₂, —CONH₂, —COOH, or
C₁₋₁₆alkylcarboxylate;
and R3 is selected from —OH or C₁₋₆alkoxy;
provided that when M is a bond and R3 is —OH, then R2 is not —C₁₋₆alkyl,
and that when M is —C(O)— then R2 is not hydrogen or —CH₃,
and with the proviso that the compound is not
2-hydroxy-3-isopropyl-6-methyl-5-[(4-nitrophenyl)sulfinyl]benzoic acid,
2-hydroxy-3-isopropyl-6-methyl-5-[(4-nitrophenyl)sulfonyl]benzoic acid,
2-hydroxy-3-isopropyl-6-methyl-5-[(4-nitrophenyl)thio]benzoic acid,
2-hydroxy-3-methyl-5-[(4-methylphenyl)sulfonyl]benzoic acid,
2-hydroxy-3-methyl-5-[(4-nitrophenyl)sulfinyl]benzoic acid,
2-hydroxy-3-methyl-5-[(4-nitrophenyl)sulfonyl]benzoic acid,
2-hydroxy-3-methyl-5-[(4-nitrophenyl)thio]benzoic acid,
3-[(4-bromo-3-methylphenyl)sulfonyl]-5-tert-butyl-6-hydroxy-2-methylbenzoic acid,
3-[(4-bromo-3-methylphenyl)thio]-5-tert-butyl-6-hydroxy-2-methylbenzoic acid,
3-[(4-bromophenyl)sulfonyl]-5-tert-butyl-6-hydroxy-2-methylbenzoic acid,
3-[(4-bromophenyl)thio]-5-tert-butyl-6-hydroxy-2-methylbenzoic acid,
3-[(4-chlorophenyl)sulfonyl]-6-hydroxy-5-isopropyl-2-methylbenzoic acid,
3-bromo-5-tert-butyl-6-hydroxy-2-methylbenzoic acid,
3-tert-butyl-2-hydroxy-5-[(4-nitrophenyl)sulfonyl]benzoic acid,
3-tert-butyl-2-hydroxy-5-[(4-nitrophenyl)thio]benzoic acid,
3-tert-butyl-2-hydroxy-6-methyl-5-(phenylsulfonyl)benzoic acid,
3-tert-butyl-2-hydroxy-6-methyl-5-(phenylthio)benzoic acid,
3-tert-butyl-2-hydroxy-6-methyl-5-[(2,4,5-trichlorophenyl)sulfinyl]benzoic acid,
3-tert-butyl-2-hydroxy-6-methyl-5-[(2,4,5-trichlorophenyl)sulfonyl]benzoic acid,
3-tert-butyl-2-hydroxy-6-methyl-5-[(2,4,5-trichlorophenyl)thio]benzoic acid,
3-tert-butyl-2-hydroxy-6-methyl-5-[(2-nitrophenyl)sulfonyl]benzoic acid,
3-tert-butyl-2-hydroxy-6-methyl-5-[(2-nitrophenyl)thio]benzoic acid,
3-tert-butyl-2-hydroxy-6-methyl-5-[(3-nitrophenyl)sulfonyl]benzoic acid,
3-tert-butyl-2-hydroxy-6-methyl-5-[(3-nitrophenyl)thio]benzoic acid,
3-tert-butyl-2-hydroxy-6-methyl-5-[(4-methylphenyl)sulfonyl]benzoic acid,
3-tert-butyl-2-hydroxy-6-methyl-5-[(4-methylphenyl)thio]benzoic acid,
3-tert-butyl-2-hydroxy-6-methyl-5-[(4-nitrophenyl)sulfinyl]benzoic acid,
3-tert-butyl-2-hydroxy-6-methyl-5-[(4-nitrophenyl)sulfonyl]benzoic acid,
3-tert-butyl-2-hydroxy-6-methyl-5-[(4-nitrophenyl)thio]benzoic acid,
3-tert-butyl-5-(4-chlorobenzoyl)-2-hydroxy-6-methylbenzoic acid,
3-tert-butyl-5-[(2,4-dinitrophenyl)sulfinyl]-2-hydroxy-6-methylbenzoic acid,
3-tert-butyl-5-[(2,4-dinitrophenyl)sulfonyl]-2-hydroxy-6-methylbenzoic acid,
3-tert-butyl-5-[(2,4-dinitrophenyl)thio]-2-hydroxy-6-methylbenzoic acid,
3-tert-butyl-5-[(2,5-dichlorophenyl)sulfonyl]-2-hydroxy-6-methylbenzoic acid,
3-tert-butyl-5-[(2,5-dichlorophenyl)thio]-2-hydroxy-6-methylbenzoic acid,
3-tert-butyl-5-[(2-chloro-5-nitrophenyl)sulfinyl]-2-hydroxy-6-methylbenzoic acid,
3-tert-butyl-5-[(2-chloro-5-nitrophenyl)sulfonyl]-2-hydroxy-6-methylbenzoic acid,
3-tert-butyl-5-[(2-chloro-5-nitrophenyl)thio]-2-hydroxy-6-methylbenzoic acid,
3-tert-butyl-5-[(3,4-dichlorophenyl)sulfonyl]-2-hydroxy-6-methylbenzoic acid,
3-tert-butyl-5-[(3,4-dichlorophenyl)thio]-2-hydroxy-6-methylbenzoic acid,
3-tert-butyl-5-[(4-chloro-2-nitrophenyl)sulfonyl]-2-hydroxy-6-methylbenzoic acid,
3-tert-butyl-5-[(4-chloro-2-nitrophenyl)thio]-2-hydroxy-6-methylbenzoic acid,
3-tert-butyl-5-[(4-chloro-3-nitrophenyl)sulfonyl]-2-hydroxy-6-methylbenzoic acid,
3-tert-butyl-5-[(4-chloro-3-nitrophenyl)thio]-2-hydroxy-6-methylbenzoic acid,
3-tert-butyl-5-[(4-chlorophenyl)sulfonyl]-2-hydroxy-6-methylbenzoic acid,
3-tert-butyl-5-[(4-chlorophenyl)thio]-2-hydroxy-6-methylbenzoic acid,
5-[(2,4-dinitrophenyl)sulfonyl]-2-hydroxy-3-methylbenzoic acid,
5-[(4-bromophenyl)sulfonyl]-2-hydroxy-3-methylbenzoic acid,
5-[(4-chlorophenyl)sulfonyl]-2-hydroxy-3-methylbenzoic acid,
5-[(4-chlorophenyl)thio]-2-hydroxy-3-methylbenzoic acid, or
3-tert-butyl-2-hydroxy-5-iodo-6-methylbenzoic acid.

43. A compound of claim 42 wherein Y is selected from hydrogen, —OH, —OC₁₋₆alkyl, or —C₁₋₆alkyl.

44. A compound of claim 42 wherein Y is selected from hydrogen, —OH, —CH₃, or —OCH₃.

45. A compound of claim 42 wherein Y is selected from —OH, —CH₃, or —OCH₃.

46. A compound of claim 42 wherein R1 is selected from aryl, heteroaryl, —C₃₋₆cycloalkyl or —C₃₋₄-alkyl.

47. A compound of claim 42 wherein R1 is selected from phenyl, pyridyl, —C₃₋₄-alkyl or cyclohexyl.

48. A compound of claim 42 wherein R1 is selected from —C₃₋₆cycloalkyl or —C₃₋₄-alkyl.

49. A compound of claim 42 wherein R1 is selected from —C₃₋₄-alkyl or cyclohexyl.

50. A compound of claim 42 wherein M is selected from —C(O)—, —C(H₂)—, —CH(OC₂H₅)—, —S(O)₂—, —S—, —N(OH)—, —N(H)—, —N(CH₃)—, oxadiazolyl, or a bond.

51. A compound of claim 42 wherein M is selected from —C(O)—, —C(H₂)—, —CH(OC₂H₅)—, —S(O)₂—, —S—, —N(OH)—, —N(H)—, —N(CH₃)—, or oxadiazolyl.

52. A compound of claim 42 wherein R2 is selected from hydrogen, halo, or —CN.

53. A compound of claim 42 wherein R2 is a group D selected from phenyl, cyclohexyl, pyridinyl, benzyl, thiazolyl, naphthyl, —N(CH₃)₂, quinoxalinyl, —CN, oxypyridinyl, —CH₃, t-butyl, propyl, thiophenyl, or dioxido-benzothienyl.

54. A compound of claim 42 wherein G is selected from —NH₂, —CONH₂, —Br, —Cl, —CN, —F, —OH, —I, —OCH₃, —NO₂, t-butyl, —COOH, —COOCH₃, —OCF₃, isopropyl, phenyl, —CH₃, —C₂H₅, morpholinyl, pyridinyl, benzothiazolyl, or —CF₃.

55. A compound of claim 42 wherein R3 is —OH or —OCH₃.

56. A compound of claim 42 wherein Y is selected from hydrogen, —OH, —CH₃, or —OCH₃;
R1 is selected from phenyl, pyridyl, —C₃₋₄-alkyl or cyclohexyl;
M is selected from —C(O)—, —C(H₂)—, —CH(OC₂H₅)—, —S(O)₂—, —S—, —N(OH)—, —N(H)—, —N(CH₃)—, oxadiazolyl, or a bond;
R2 is selected from hydrogen, halo, or —CN;
D is selected from phenyl, cyclohexyl, pyridinyl, benzyl, thiazolyl, naphthyl, —N(CH₃)₂, quinoxalinyl, —CN, oxypyridinyl, —CH₃, t-butyl, propyl, thiophenyl, or dioxido-benzothienyl;
G is selected from —NH₂, —CONH₂, —Br, —Cl, —CN, —F, —OH, —I, —OCH₃, —NO₂, t-butyl, —COOH, —COOCH₃, —OCF₃, isopropyl, phenyl, —CH₃, —C₂H₅, morpholinyl, pyridinyl, benzothiazolyl, or —CF₃; and
R3 is —OH or —OCH₃.

57. A compound selected from the group consisting of:
3-tert-butyl-5-(4-chloro-3-iodobenzoyl)-2-hydroxy-6-methylbenzoic acid,
3-tert-Butyl-5-(4-tert-Butyl-benzoyl)-2-hydroxy-6-methyl-benzoic acid,
3-tert-Butyl-5-(4-trifluoromethoxy-benzoyl)-2-hydroxy-6-methyl-benzoic acid,
3-benzoyl-5-tert-butyl-6-hydroxy-2-methylbenzoic acid,
3-tert-butyl-5-(4-chloro-2-fluorobenzoyl)-2-hydroxy-6-methylbenzoic acid,
3-tert-butyl-5-(4-chloro-3-fluorobenzoyl)-2-hydroxy-6-methylbenzoic acid,
3-tert-Butyl-2,6-dihydroxy-benzoic acid,
3-tert-Butyl-5-(4-chloro-benzoyl)-2,6-dihydroxy-benzoic acid,
3-tert-butyl-5-(3,4-difluoro-benzoyl)-2,6-dihydroxy-benzoic acid,
3-tert-butyl-2,6-dihydroxy-5-(quinoxalin-2-ylcarbonyl)benzoic acid,
3-(4-chloro-benzoyl)-5-cyclohexyl-2,6-dihydroxy-benzoic acid,
3-tert-Butyl-5-[(4-chloro-phenyl)-hydroxyimino-methyl]-2-hydroxy-6-methyl-benzoic acid,
5,5′-di-tert-butyl-4,4′-dihydroxy-3′-(methoxycarbonyl)-2,2′-dimethylbiphenyl-3-carboxylic acid,
3-tert-Butyl-5-(4-fluorobenzoyl)-2-hydroxy-6-methylbenzoic acid,
3-tert-butyl-2-hydroxy-6-methyl-5-(4-methylbenzoyl)benzoic acid,
3-tert-butyl-5-(3,4-dichlorobenzoyl)-2-hydroxy-6-methylbenzoic acid,
3-tert-butyl-2-hydroxy-6-methyl-5-[4-(trifluoromethyl)benzoyl]benzoic acid,
3-tert-butyl-5-(2,4-dichlorobenzoyl)-2-hydroxy-6-methylbenzoic acid,
3-tert-butyl-2-hydroxy-6-methyl-5-[3-(trifluoromethoxy)benzoyl]benzoic acid,
3-tert-butyl-2-hydroxy-5-(3-isopropylbenzoyl)-6-methylbenzoic acid,
3-tert-butyl-2-hydroxy-6-methyl-5-(3-nitrobenzoyl)benzoic acid,
3-tert-butyl-2-hydroxy-5-(2-hydroxybenzoyl)-6-methylbenzoic acid,
3-tert-butyl-2-hydroxy-6-methyl-5-[2-(trifluoromethyl)benzoyl]benzoic acid,
5-tert-butyl-4-hydroxy-2-methylbiphenyl-3-carboxylic acid,
5-tert-butyl-4-hydroxy-2,2′-dimethylbiphenyl-3-carboxylic acid,
5-tert-butyl-4-hydroxy-4′-methoxy-2,2′-dimethylbiphenyl-3-carboxylic acid,
5-tert-butyl-4-hydroxy-2,2′-dimethylbiphenyl-3-carboxylic acid,
5-tert-butyl-4-hydroxy-4′-methoxy-2-methylbiphenyl-3-carboxylic acid,
5-tert-butyl-4-hydroxy-3′-isopropyl-2-methylbiphenyl-3-carboxylic acid,
3′,5-di-tert-butyl-4-hydroxy-2,5′-dimethylbiphenyl-3-carboxylic acid,
3-anilino-5-tert-butyl-6-hydroxy-2-methylbenzoic acid,
3-tert-butyl-5-[(4-chlorophenyl)amino]-2-hydroxy-6-methylbenzoic acid,
3-tert-butyl-5-[(4-chlorophenyl)(methyl)amino]-2-hydroxy-6-methylbenzoic acid,
3-tert-Butyl-5-[5-(4-chlorophenyl)-[1,2,4]oxadiazol-3-yl]-2-hydroxy-6-methylbenzoic acid,
3-tert-butyl-2-hydroxy-5-[(4-methoxyphenyl)thio]-6-methylbenzoic acid,
3-tert-butyl-2-hydroxy-6-methyl-5-(1-naphthylthio)benzoic acid,
3-[(2,4-dichlorophenyl)thio]-6-hydroxy-5-isopropyl-2-methylbenzoic acid,
3-tert-butyl-5-[(2,4-dichlorophenyl)thio]-2,6-dihydroxybenzoic acid,
2-hydroxy-3-isopropyl-6-methyl-5-(1-naphthylthio)benzoic acid,
3-tert-butyl-2-hydroxy-6-methyl-5-[(4-phenyl-1,3-thiazol-2-yl)thio]benzoic acid,
3-tert-butyl-2,6-dihydroxy-5-(1-naphthylthio)benzoic acid,
3-tert-butyl-5-[(2,4-dichlorophenyl)thio]-2-hydroxy-6-methylbenzoic acid,
3-(benzylthio)-5-tert-butyl-6-hydroxy-2-methylbenzoic acid,
3-tert-butyl-5-[(2,3-difluorobenzyl)thio]-2-hydroxy-6-methylbenzoic acid,
3-tert-butyl-5-[(4-chlorobenzyl)thio]-2-hydroxy-6-methylbenzoic acid,
3-(benzylsulfinyl)-5-tert-butyl-6-hydroxy-2-methylbenzoic acid,
3-(benzylsulfonyl)-5-tert-butyl-6-hydroxy-2-methylbenzoic acid,
3-tert-butyl-2-hydroxy-5-[(4-methoxyphenyl)sulfonyl]-6-methylbenzoic acid,
3-tert-butyl-2-hydroxy-6-methyl-5-(1-naphthylsulfonyl)benzoic acid,
3-tert-butyl-5-[(2,4-dichlorophenyl)sulfonyl]-2,6-dihydroxybenzoic acid,
3-[(2,4-dichlorophenyl)sulfonyl]-6-hydroxy-5-isopropyl-2-methylbenzoic acid,
3-tert-butyl-5-[(2,4-dichlorophenyl)sulfonyl]-2-hydroxy-6-methylbenzoic acid,
3-tert-butyl-5-[(4-chlorophenyl)(ethoxy)methyl]-2-hydroxy-6-methylbenzoic acid,
3,5-di-tert-butyl-2,6-dimethoxybenzoic acid,
3-tert-butyl-5-[(2,3-difluorobenzyl)thio]-2-hydroxy-6-methylbenzoic acid,
3-tert-butyl-2-hydroxy-6-methyl-5-(pyridin-4-ylthio)benzoic acid,
2-hydroxy-3-isopropyl-6-methyl-5-(1-naphthylsulfonyl)benzoic acid,
3-tert-butyl-5-{[(5-fluoro-1,3-benzothiazol-2-yl)methyl]thio}-2-hydroxy-6-methylbenzoic acid,
3-tert-butyl-2-hydroxy-5-[(3-methoxybenzyl)thio]-6-methylbenzoic acid,
3-tert-butyl-5-[(2-cyanobenzyl)thio]-2-hydroxy-6-methylbenzoic acid,
3-tert-butyl-2-hydroxy-6-methyl-5-[(tetrahydro-2H-pyran-2-ylmethyl)thio]benzoic acid,
3-tert-butyl-2-hydroxy-6-methyl-5-[(pyridin-3-ylmethyl)thio]benzoic acid,
3-tert-butyl-2-hydroxy-6-methyl-5-[(pyridin-4-ylmethyl)thio]benzoic acid,
3-tert-butyl-2-hydroxy-5-(isobutylthio)-6-methylbenzoic acid,
3-tert-butyl-2-hydroxy-6-methyl-5-[(2-phenylethyl)thio]benzoic acid,
3-tert-butyl-2-hydroxy-6-methyl-5-{[2-(trifluoromethyl)benzyl]thio}-benzoic acid,
3-tert-butyl-5-[(2,3-difluorobenzyl)sulfonyl]-2-hydroxy-6-methylbenzoic acid,
3-tert-butyl-5-[(4-chlorobenzyl)sulfonyl]-2-hydroxy-6-methylbenzoic acid,
3-tert-butyl-2-hydroxy-6-methyl-5-[(pyridin-2-ylmethyl)sulfonyl]benzoic acid,
3-tert-butyl-2-hydroxy-6-methyl-5-[(3-methylbenzyl)sulfonyl]benzoic acid,
3-tert-butyl-2-hydroxy-6-methyl-5-[(3-methylbenzyl)thio]benzoic acid,
3-tert-butyl-2-hydroxy-6-methyl-5-{[2-(trifluoromethyl)benzyl]sulfonyl}-benzoic acid,
3-tert-butyl-2-hydroxy-6-methyl-5-(phenylacetyl)benzoic acid,
3-tert-butyl-2-hydroxy-6-methyl-5-[phenyl(phenylthio)acetyl]benzoic acid,
3,5-Di-tert-butyl-2-chloro-6-hydroxybenzoic acid,
3-tert-butyl-5-[(3,4-difluorophenyl)thio]-2-hydroxy-6-methylbenzoic acid,
3-tert-butyl-5-[(3,4-difluorophenyl)sulfonyl]-2-hydroxy-6-methylbenzoic acid,
3-tert-butyl-2-hydroxy-6-methyl-5-[(2,4,5-trichlorophenyl)sulfonyl]benzoic acid,
3-tert-butyl-2-hydroxy-6-methyl-5-{[4-(trifluoromethoxy)phenyl]sulfonyl}benzoic acid,
3-{[3,5-Bis(trifluoromethyl)phenyl]sulfonyl}-5-tert-butyl-6-hydroxy-2-methylbenzoic acid,
3-tert-butyl-5-[(2,6-dichlorophenyl)sulfonyl]-2-hydroxy-6-methylbenzoic acid,
3-tert-butyl-5-[(2,3-dichlorophenyl)sulfonyl]-2-hydroxy-6-methylbenzoic acid,
3-tert-butyl-5-[(2-chloro-4-fluorophenyl)sulfonyl]-2-hydroxy-6-methylbenzoic acid,
3-tert-butyl-5-[(3-chloro-4-fluorophenyl)sulfonyl]-2-hydroxy-6-methylbenzoic acid,
3-tert-butyl-5-[(3,5-dichlorophenyl)sulfonyl]-2-hydroxy-6-methylbenzoic acid,
3′-tert-butyl-4-hydroxy-5′-methyl-5-pyridin-3-ylbiphenyl-3-carboxylic acid,
3-(1-Benzofuran-2-yl)-5-tert-butyl-6-hydroxy-2-methylbenzoic acid,
3-tert-butyl-5-(1,1-dioxido-1-benzothien-2-yl)-2-hydroxy-6-methylbenzoic acid,
5-tert-butyl-3′,4′-dichloro-4-hydroxy-2-methylbiphenyl-3-carboxylic acid,
5-tert-butyl-2′,4′-dichloro-4-hydroxy-2-methylbiphenyl-3-carboxylic acid,
5-tert-butyl-4-hydroxy-2-methyl-4′-morpholin-4-ylbiphenyl-3-carboxylic acid,
3-tert-butyl-2-hydroxy-6-methyl-5-(1-naphthyl)benzoic acid,
5-tert-butyl-3′-cyano-4-hydroxy-2-methylbiphenyl-3-carboxylic acid,
5-tert-butyl-4-hydroxy-2-methyl-3′,5′-bis(trifluoromethyl)biphenyl-3-carboxylic acid,
3-tert-butyl-2-hydroxy-6-methyl-5-(2-naphthyl)benzoic acid,
3-tert-butyl-2-hydroxy-5-isoquinolin-4-yl-6-methylbenzoic acid,
3-tert-butyl-2-hydroxy-6-methyl-5-quinolin-3-ylbenzoic acid,
3-tert-butyl-2-hydroxy-6-methyl-5-quinolin-8-ylbenzoic acid,
3-tert-butyl-2-hydroxy-6-methyl-5-quinolin-6-ylbenzoic acid,
3-tert-butyl-2-hydroxy-6-methyl-5-quinolin-5-ylbenzoic acid,
4′-hydroxy-6′-methoxy-1,1′:3′,1″-terphenyl-5′-carboxylic acid,
4,4″-difluoro-4′-hydroxy-1,1′:3′,1″-terphenyl-5′-carboxylic acid,
3-tert-butyl-4′-hydroxy-5-methyl-1,1′:3′,1″-terphenyl-5′-carboxylic acid, and
2,6-dihydroxy-3,5-diisopropylbenzoic acid.

58. A pharmaceutical composition comprising as active ingredient a therapeutically effective amount of the compound of formula I, or pharmaceutically acceptable salts thereof 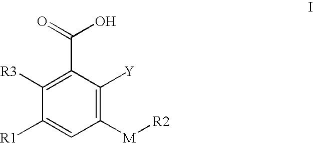
wherein
Y is selected from hydrogen, —OH, halo, —OC₁₋₆alkyl, or —C₁₋₆alkyl, the two latter optionally substituted with halo, —CN, —OH, —CF₃, —NH₂;
R1 is selected from —C₃₋₆cycloalkyl, heterocycloalkyl, aryl, alkylaryl, heteroaryl, or —C₃₋₆alkyl, optionally substituted with halo, —CN, —OH, —CF₃, —OCF₃, —NH₂, —CONH₂;
M is selected from —C(O)—, —C(H₂)—, —CH(OR^{a})—, —N(OH)—, —N(R^{a})—, —S(O)ᵣ—, heteroaryl or a bond;
wherein R^{a} is hydrogen or C₁₋₆alkyl and r is 0, 1 or 2;
R2 is either selected from hydrogen, halo, —CN, or is a group D selected from —C₁₋₆alkyl, C₃₋₆cycloalkyl, heterocycloalkyl, —N(CH₃)₂, aryl, alkylaryl, heteroaryl, or a heterocyclic group, where D is optionally substituted with one or more substituents G selected from halo, —NO₂, —CN, —OH, —CF₃, —OCF₃, —NH₂, —CONH₂, —COOH, aryl, heteroaryl, heterocyclic groups, —C₁₋₆alkyl, —C₁₋₆alkoxy, heterocycloalkyl, or C₁₋₆alkylcarboxylate; where D may optionally be connected to G by a linker group L selected from —C(O)—, —S—, or —S(O₂)—; and G, if substitutable, is optionally further substituted with one or more substituents selected from halo, —NO₂, —CN, —OH, —CH₃, —OCH₃, —CF₃, —OCF₃, —NH₂, —CONH₂, —COOH, or C₁₋₆alkylcarboxylate;
and R3 is selected from —OH or C₁₋₆alkoxy;
provided that when M is a bond and R3 is —OH, then R2 is not —C₁₋₆alkyl,
in association with one or more pharmaceutically acceptable diluents, excipients or inert carriers.

59. A pharmaceutical composition of claim 58 wherein Y is selected from hydrogen, —OH, —OC₁₋₆alkyl, or —C₁₋₆alkyl.

60. A pharmaceutical composition of claim 58 wherein Y is selected from hydrogen, —OH, —CH₃, or —OCH₃.

61. A pharmaceutical composition of claim 58 wherein Y is selected from —OH, —CH₃, or —OCH₃.

62. A pharmaceutical composition of claim 58 wherein R1 is selected from aryl, heteroaryl, —C₃₋₆cycloalkyl or —C₃₋₄-alkyl.

63. A pharmaceutical composition of claim 58 wherein R1 is selected from phenyl, pyridyl, —C₃₋₄-alkyl or cyclohexyl.

64. A pharmaceutical composition of claim 58 wherein R1 is selected from —C₃₋₆cycloalkyl or —C₃₋₄-alkyl.

65. A pharmaceutical composition of claim 58 wherein R1 is selected from —C₃₋₄-alkyl or cyclohexyl.

66. A pharmaceutical composition of claim 58 wherein M is selected from —C(O)—, —C(H₂)—, —C(OC₂H₅)—, —S(O)₂—, —S—, —N(OH)—, —N(H)—, —N(CH₃)—, oxadiazolyl, or a bond.

67. A pharmaceutical composition of claim 58 wherein R2 is selected from phenyl, cyclohexyl, pyridinyl, benzyl, bromo, thiazolyl, naphthyl, quinoxalinyl, oxypyridinyl, propyl, thiophenyl, or dioxido-benzothienyl.

68. A pharmaceutical composition of claim 58 wherein G is selected from —NH₂, —CONH₂, —Br, —Cl, —CN, —F, —OH, —I, —OCH₃, —NO₂, t-butyl, —COOH, —COOCH₃, —OCF₃, isopropyl, phenyl, —CH₃, —C₂H₅, morpholinyl, pyridinyl, benzothiazolyl, or —CF₃.

69. A pharmaceutical composition of claim 58 wherein R3 is —OH or —OCH₃.

70. A pharmaceutical composition of claim 58 wherein Y is selected from hydrogen, —OH, —CH₃, or —OCH₃;
R1 is selected from aryl, heteroaryl, —C₃₋₄-alkyl or cyclohexyl;
M is selected from —C(O)—, —C(H₂)—, —C(OC₂H₅)—, —S(O)₂—, —S—, —N(OH)—, —N(H)—, —N(CH₃)—, oxadiazolyl, or a bond;
R2 is selected from phenyl, cyclohexyl, pyridinyl, benzyl, bromo, thiazolyl, naphthyl, quinoxalinyl, oxypyridinyl, propyl, thiophenyl, or dioxido-benzothienyl;
G is selected from —NH₂, —CONH₂, —Br, —Cl, —CN, —F, —OH, —I, —OCH₃, —NO₂, t-butyl, —COOH, —COOCH₃, —OCF₃, isopropyl, phenyl, —CH₃, —C₂H₅, morpholinyl, pyridinyl, benzothiazolyl, or —CF₃; and
R3 is —OH or —OCH₃.

71. A pharmaceutical composition of claim 58 in which the active ingredient is selected from the group consisting of
2-hydroxy-3-isopropyl-6-methyl-5-[(4-nitrophenyl)sulfinyl]benzoic acid,
2-hydroxy-3-isopropyl-6-methyl-5-[(4-nitrophenyl)sulfonyl]benzoic acid,
2-hydroxy-3-isopropyl-6-methyl-5-[(4-nitrophenyl)thio]benzoic acid,
2-hydroxy-3-methyl-5-[(4-methylphenyl)sulfonyl]benzoic acid,
2-hydroxy-3-methyl-5-[(4-nitrophenyl)sulfinyl]benzoic acid,
2-hydroxy-3-methyl-5-[(4-nitrophenyl)sulfonyl]benzoic acid,
2-hydroxy-3-methyl-5-[(4-nitrophenyl)thio]benzoic acid,
3-[(4-bromo-3-methylphenyl)sulfonyl]-5-tert-butyl-6-hydroxy-2-methylbenzoic acid,
3-[(4-bromo-3-methylphenyl)thio]-5-tert-butyl-6-hydroxy-2-methylbenzoic acid,
3-[(4-bromophenyl)sulfonyl]-5-tert-butyl-6-hydroxy-2-methylbenzoic acid,
3-[(4-bromophenyl)thio]-5-tert-butyl-6-hydroxy-2-methylbenzoic acid,
3-[(4-chlorophenyl)sulfonyl]-6-hydroxy-5-isopropyl-2-methylbenzoic acid,
3-bromo-5-tert-butyl-6-hydroxy-2-methylbenzoic acid,
3-tert-butyl-2-hydroxy-5-[(4-nitrophenyl)sulfonyl]benzoic acid,
3-tert-butyl-2-hydroxy-5-[(4-nitrophenyl)thio]benzoic acid,
3-tert-butyl-2-hydroxy-6-methyl-5-(phenylsulfonyl)benzoic acid,
3-tert-butyl-2-hydroxy-6-methyl-5-(phenylthio)benzoic acid,
3-tert-butyl-2-hydroxy-6-methyl-5-[(2,4,5-trichlorophenyl)sulfinyl]benzoic acid,
3-tert-butyl-2-hydroxy-6-methyl-5-[(2,4,5-trichlorophenyl)sulfonyl]benzoic acid,
3-tert-butyl-2-hydroxy-6-methyl-5-[(2,4,5-trichlorophenyl)thio]benzoic acid,
3-tert-butyl-2-hydroxy-6-methyl-5-[(2-nitrophenyl)sulfonyl]benzoic acid,
3-tert-butyl-2-hydroxy-6-methyl-5-[(2-nitrophenyl)thio]benzoic acid,
3-tert-butyl-2-hydroxy-6-methyl-5-[(3-nitrophenyl)sulfonyl]benzoic acid,
3-tert-butyl-2-hydroxy-6-methyl-5-[(3-nitrophenyl)thio]benzoic acid,
3-tert-butyl-2-hydroxy-6-methyl-5-[(4-methylphenyl)sulfonyl]benzoic acid,
3-tert-butyl-2-hydroxy-6-methyl-5-[(4-methylphenyl)thio]benzoic acid,
3-tert-butyl-2-hydroxy-6-methyl-5-[(4-nitrophenyl)sulfinyl]benzoic acid,
3-tert-butyl-2-hydroxy-6-methyl-5-[(4-nitrophenyl)sulfonyl]benzoic acid,
3-tert-butyl-2-hydroxy-6-methyl-5-[(4-nitrophenyl)thio]benzoic acid,
3-tert-butyl-5-(4-chlorobenzoyl)-2-hydroxy-6-methylbenzoic acid,
3-tert-butyl-5-[(2,4-dinitrophenyl)sulfinyl]-2-hydroxy-6-methylbenzoic acid,
3-tert-butyl-5-[(2,4-dinitrophenyl)sulfonyl]-2-hydroxy-6-methylbenzoic acid,
3-tert-butyl-5-[(2,4-dinitrophenyl)thio]-2-hydroxy-6-methylbenzoic acid,
3-tert-butyl-5-[(2,5-dichlorophenyl)sulfonyl]-2-hydroxy-6-methylbenzoic acid,
3-tert-butyl-5-[(2,5-dichlorophenyl)thio]-2-hydroxy-6-methylbenzoic acid,
3-tert-butyl-5-[(2-chloro-5-nitrophenyl)sulfinyl]-2-hydroxy-6-methylbenzoic acid,
3-tert-butyl-5-[(2-chloro-5-nitrophenyl)sulfonyl]-2-hydroxy-6-methylbenzoic acid,
3-tert-butyl-5-[(2-chloro-5-nitrophenyl)thio]-2-hydroxy-6-methylbenzoic acid,
3-tert-butyl-5-[(3,4-dichlorophenyl)sulfonyl]-2-hydroxy-6-methylbenzoic acid,
3-tert-butyl-5-[(3,4-dichlorophenyl)thio]-2-hydroxy-6-methylbenzoic acid,
3-tert-butyl-5-[(4-chloro-2-nitrophenyl)sulfonyl]-2-hydroxy-6-methylbenzoic acid,
3-tert-butyl-5-[(4-chloro-2-nitrophenyl)thio]-2-hydroxy-6-methylbenzoic acid,
3-tert-butyl-5-[(4-chloro-3-nitrophenyl)sulfonyl]-2-hydroxy-6-methylbenzoic acid,
3-tert-butyl-5-[(4-chloro-3-nitrophenyl)thio]-2-hydroxy-6-methylbenzoic acid,
3-tert-butyl-5-[(4-chlorophenyl)sulfonyl]-2-hydroxy-6-methylbenzoic acid,
3-tert-butyl-5-[(4-chlorophenyl)thio]-2-hydroxy-6-methylbenzoic acid,
5-[(2,4-dinitrophenyl)sulfonyl]-2-hydroxy-3-methylbenzoic acid,
5-[(4-bromophenyl)sulfonyl]-2-hydroxy-3-methylbenzoic acid,
5-[(4-chlorophenyl)sulfonyl]-2-hydroxy-3-methylbenzoic acid,
5-[(4-chlorophenyl)thio]-2-hydroxy-3-methylbenzoic acid,
3-tert-butyl-2-hydroxy-5-iodo-6-methylbenzoic acid,
3-tert-butyl-5-[(2,3-difluorobenzyl)thio]-2-hydroxy-6-methylbenzoic acid,
3-tert-butyl-2-hydroxy-6-methyl-5-(pyridin-4-ylthio)benzoic acid,
2-hydroxy-3-isopropyl-6-methyl-5-(1-naphthylsulfonyl)benzoic acid,
3-tert-butyl-5-{[(5-fluoro-1,3-benzothiazol-2-yl)methyl]thio}-2-hydroxy-6-methylbenzoic acid,
3-tert-butyl-2-hydroxy-5-[(3-methoxybenzyl)thio]-6-methylbenzoic acid,
3-tert-butyl-5-[(2-cyanobenzyl)thio]-2-hydroxy-6-methylbenzoic acid,
3-tert-butyl-2-hydroxy-6-methyl-5-[(tetrahydro-2H-pyran-2-ylmethyl)thio]benzoic acid,
3-tert-butyl-2-hydroxy-6-methyl-5-[(pyridin-3-ylmethyl)thio]benzoic acid,
3-tert-butyl-2-hydroxy-6-methyl-5-[(pyridin-4-ylmethyl)thio]benzoic acid,
3-tert-butyl-2-hydroxy-5-(isobutylthio)-6-methylbenzoic acid,
3-tert-butyl-2-hydroxy-6-methyl-5-[(2-phenylethyl)thio]benzoic acid,
3-tert-butyl-2-hydroxy-6-methyl-5-{[2-(trifluoromethyl)benzyl]thio}-benzoic acid,
3-tert-butyl-5-[(2,3-difluorobenzyl)sulfonyl]-2-hydroxy-6-methylbenzoic acid,
3-tert-butyl-5-[(4-chlorobenzyl)sulfonyl]-2-hydroxy-6-methylbenzoic acid,
3-tert-butyl-2-hydroxy-6-methyl-5-[(pyridin-2-ylmethyl)sulfonyl]benzoic acid,
3-tert-butyl-2-hydroxy-6-methyl-5-[(3-methylbenzyl)sulfonyl]benzoic acid,
3-tert-butyl-2-hydroxy-6-methyl-5-[(3-methylbenzyl)thio]benzoic acid,
3-tert-butyl-2-hydroxy-6-methyl-5-{[2-(trifluoromethyl)benzyl]sulfonyl}-benzoic acid,
3-tert-butyl-2-hydroxy-6-methyl-5-(phenylacetyl)benzoic acid,
3-tert-butyl-2-hydroxy-6-methyl-5-[phenyl(phenylthio)acetyl]benzoic acid,
3,5-Di-tert-butyl-2-chloro-6-hydroxybenzoic acid,
3-tert-butyl-5-[(3,4-difluorophenyl)thio]-2-hydroxy-6-methylbenzoic acid,
3-tert-butyl-5-[(3,4-difluorophenyl)sulfonyl]-2-hydroxy-6-methylbenzoic acid,
3-tert-butyl-2-hydroxy-6-methyl-5-[(2,4,5-trichlorophenyl)sulfonyl]benzoic acid,
3-tert-butyl-2-hydroxy-6-methyl-5-{[4-(trifluoromethoxy)phenyl]sulfonyl}benzoic acid,
3-{[3,5-Bis(trifluoromethyl)phenyl]sulfonyl}-5-tert-butyl-6-hydroxy-2-methylbenzoic acid,
3-tert-butyl-5-[(2,6-dichlorophenyl)sulfonyl]-2-hydroxy-6-methylbenzoic acid,
3-tert-butyl-5-[(2,3-dichlorophenyl)sulfonyl]-2-hydroxy-6-methylbenzoic acid,
3-tert-butyl-5-[(2-chloro-4-fluorophenyl)sulfonyl]-2-hydroxy-6-methylbenzoic acid,
3-tert-butyl-5-[(3-chloro-4-fluorophenyl)sulfonyl]-2-hydroxy-6-methylbenzoic acid,
3-tert-butyl-5-[(3,5-dichlorophenyl)sulfonyl]-2-hydroxy-6-methylbenzoic acid,
3′-tert-butyl-4-hydroxy-5′-methyl-5-pyridin-3-ylbiphenyl-3-carboxylic acid,
3-(1-Benzofuran-2-yl)-5-tert-butyl-6-hydroxy-2-methylbenzoic acid,
3-tert-butyl-5-(1,1-dioxido-1-benzothien-2-yl)-2-hydroxy-6-methylbenzoic acid,
5-tert-butyl-3′,4′-dichloro-4-hydroxy-2-methylbiphenyl-3-carboxylic acid,
5-tert-butyl-2′,4′-dichloro-4-hydroxy-2-methylbiphenyl-3-carboxylic acid,
5-tert-butyl-4-hydroxy-2-methyl-4′-morpholin-4-ylbiphenyl-3-carboxylic acid,
3-tert-butyl-2-hydroxy-6-methyl-5-(1-naphthyl)benzoic acid,
5-tert-butyl-3′-cyano-4-hydroxy-2-methylbiphenyl-3-carboxylic acid,
5-tert-butyl-4-hydroxy-2-methyl-3′,5′-bis(trifluoromethyl)biphenyl-3-carboxylic acid,
3-tert-butyl-2-hydroxy-6-methyl-5-(2-naphthyl)benzoic acid,
3-tert-butyl-2-hydroxy-5-isoquinolin-4-yl-6-methylbenzoic acid,
3-tert-butyl-2-hydroxy-6-methyl-5-quinolin-3-ylbenzoic acid,
3-tert-butyl-2-hydroxy-6-methyl-5-quinolin-8-ylbenzoic acid,
3-tert-butyl-2-hydroxy-6-methyl-5-quinolin-6-ylbenzoic acid,
3-tert-butyl-2-hydroxy-6-methyl-5-quinolin-5-ylbenzoic acid,
4′-hydroxy-6′-methoxy-1,1′:3′,1″-terphenyl-5′-carboxylic acid,
4,4″-difluoro-4′-hydroxy-1,1′:3′,1″-terphenyl-5′-carboxylic acid,
3-tert-butyl-4′-hydroxy-5-methyl-1,1′:3′,1″-terphenyl-5′-carboxylic acid, and
2,6-dihydroxy-3,5-diisopropylbenzoic acid.

72. A method of treating neuropathic or inflammatory pain syndromes such as painful diabetic neuropathy, post traumatic neuralgia, post herpetic neuralgia, trigeminal neuralgia, arthritis, rheumatoid diseases, fibromyalgia, low back pain with radiculopathy and post-operative pain;
pain associated with angina, renal or billiary colic, menstruation, migraine and gout, stroke, head trauma, anoxic and ischemic injuries, hypoglycaemia, cardiovascular diseases and/or cancer;
auditory neuropathic disorders such as tinnitus;
opthalmological disorders such as retinopathies, diabetic retinopathies or glaucoma;
psychiatric disorders, such as alcoholism, drug addiction and psychosis;
inflammation related diseases, such as rheumatoid arthritis and osteoarthritis;
and/or
arthrosclerosis and stroke,
comprising administering to a mammal, including man, in need of such treatment, a therapeutically effective amount of the compound according to claim 1.

73. The method according to claim 31, for treating neuropathic pain syndromes.

74. A process for preparing a compound of formula I, wherein Y, R1, R2 and R3 are, unless specified otherwise, defined as in formula I, comprising:
a) reacting an optionally protected compound of formula (II) 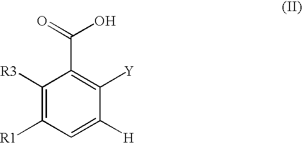
i) with a compound of formula (III) in a suitable solvent, wherein W is a halogen or a suitable leaving group, 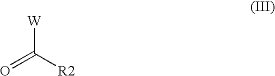
or
ii) with a compound of formula (IV) wherein W is a halogen, and n is 0, 1, or 2, in a suitable solvent, optionally followed by treatment with an oxidation reagent in case n is 0 or 1, or 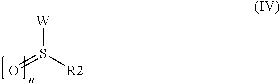
b) reacting an optionally protected compound of formula (V), wherein Hal is a halogen or a sulfonyloxy group, 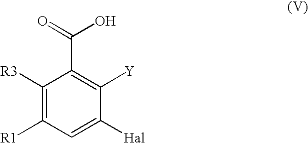
i) with an organometallic reagent of formula (VI), wherein Met is a suitable metallic group, or an organoboron reagent, 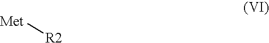
in the presence of a carbon monoxide or dry nitrogen atmosphere, and in the presence of a metallic catalyst,
or
ii) with an amine of formula (VII), 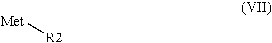
in the presence of a metallic catalyst, and in the presence of a suitable inert solvent or diluent,
or
iii) with a mercaptan of formula (X), 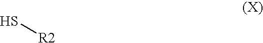
or
c) reacting an optionally protected compound of formula (VIII), 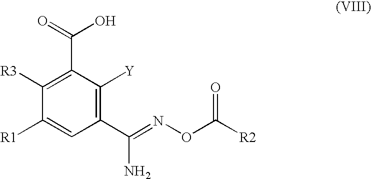
by heating in a suitable solvent between 30° C. and reflux,
or
d) reacting an optionally protected compound of formula (XV) 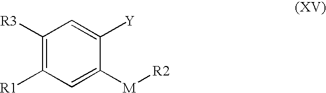
with a suitable base and carbon dioxide in a suitable solvent at a temperature between −78° C. and reflux,
or
j) reacting an optionally protected compound of formula (XVIII) 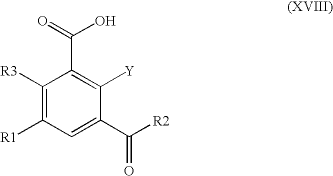
with a suitable reducing agent or by catalytic hydrogenation over a suitable catalyst in a suitable solvent,
or
k) reacting an optionally protected compound of formula (XVIII) 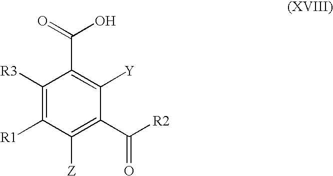
with optionally substituted hydroxylamine hydrochloride in the presence of a base in a solvent with removal of water at reflux temperature,
or
l) reacting an optionally protected compound of formula XXX 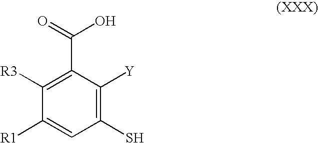
with a suitable electrophile in the presence of a suitable base in a suitable solvent at a temperature in the range from 0° C. to reflux, and thereafter optionally:
i) converting a compound of the formula I into another compound of the formula I;
and/or
ii) removing any protecting groups;
and/or
iii) forming a pharmaceutically acceptable salt.
